SEQUENCE LISTING (110 sequences)

SEQ ID NO: 1 - protein, 15 aa, Homo sapiens
GRVFVSPLAKKLAVE

SEQ ID NO: 2 - protein, 15 aa, Homo sapiens
RLLLQLLGSPGRRYY

SEQ ID NO: 3 - protein, 15 aa, Homo sapiens
DIPISNIRRVIAQRL

SEQ ID NO: 4 - protein, 15 aa, Homo sapiens
GRVFVSPLAKKLAVE

SEQ ID NO: 5 - protein, 15 aa, Homo sapiens
SPGRRYYSLPPHQKV

SEQ ID NO: 6 - protein, 15 aa, Homo sapiens
ETIANDVVSLATKAR

SEQ ID NO: 7 - protein, 15 aa, Homo sapiens
AQWLAEFRKYLEKPI

SEQ ID NO: 8 - protein, 14 aa, Homo sapiens
GDLIAEVETDKATV

SEQ ID NO: 9 - protein, 14 aa, Homo sapiens
GDLLAEIETDKATI

SEQ ID NO: 10 - protein, 15 aa, Homo sapiens
GDLLAEIETDKATIG

SEQ ID NO: 11 - protein, 15 aa, Homo sapiens
DIPISNIRRVIAQRL

SEQ ID NO: 12 - protein, 15 aa, Homo sapiens
SPGRRYYSLPPHQKV

SEQ ID NO: 13 - protein, 15 aa, Homo sapiens
GNPESSFNDENLRIV

SEQ ID NO: 14 - protein, 15 aa, Homo sapiens
GNPESSFNDENLRIV

SEQ ID NO: 15 - protein, 15 aa, Homo sapiens
SFNDENLRIVVADLF

SEQ ID NO: 16 - protein, 15 aa, Homo sapiens
LLLMILHPDVQRRVQ

SEQ ID NO: 17 - protein, 20 aa, Homo sapiens
TLAWGLLLMILHPDVQRRVQ

SEQ ID NO: 18 - protein, 20 aa, Homo sapiens
TTLITNLSSVLKDEAVWEKP

SEQ ID NO: 19 - protein, 15 aa, Homo sapiens
GRRFEYDDPRFLRLL

SEQ ID NO: 20 - protein, 15 aa, Homo sapiens
ALVPLAVIVAIFLLL

SEQ ID NO: 21 - protein, 15 aa, Homo sapiens
ENLRIVVADLFSAGM

SEQ ID NO: 22 - protein, 15 aa, Homo sapiens
FLREVLNAVPVLLHI

SEQ ID NO: 23 - protein, 15 aa, Homo sapiens
AGKVLRFQKAFLTQL

SEQ ID NO: 24 - protein, 15 aa, Homo sapiens
IFLLLVDLMHRRQRW

SEQ ID NO: 25 - protein, 15 aa, Homo sapiens
ALAGKVLRFQKAFLT

SEQ ID NO: 26 - protein, 15 aa, Homo sapiens
GLLLMILHPDVQRRV

SEQ ID NO: 27 - protein, 15 aa, Homo sapiens
ENLRIVVADLFSAGM

SEQ ID NO: 28 - protein, 15 aa, Homo sapiens
VKPEAFLPFSAGRRA

SEQ ID NO: 29 - protein, 15 aa, Homo sapiens
AGKVLRFQKAFLTQL

SEQ ID NO: 30 - protein, 15 aa, Homo sapiens
AIFLLLVDLMHRRQR

SEQ ID NO: 31 - protein, 15 aa, Homo sapiens
DPRFLRLLDLAQEGL

SEQ ID NO: 32 - protein, 15 aa, Homo sapiens
RMELFLFFTSLLQHF

SEQ ID NO: 33 - protein, 15 aa, Homo sapiens
DLFSAGMVTTSTTLA

SEQ ID NO: 34 - protein, 15 aa, Homo sapiens
ELFLFFTSLLQHFSF

SEQ ID NO: 35 - protein, 15 aa, Homo sapiens
DQLRRRFGDVFSLQL

SEQ ID NO: 36 - protein, 15 aa, Homo sapiens
EQRRFSVSTLRNLGL

SEQ ID NO: 37 - protein, 20 aa, Homo sapiens
RRFEYDDPRFLRLLDLAQEG

SEQ ID NO: 38 - protein, 20 aa, Homo sapiens
AGMVTTSTTLAWGLLLMILH

SEQ ID NO: 39 - protein, 15 aa, Homo sapiens
TTAEREIVRDIKEKL

SEQ ID NO: 40 - protein, 15 aa, Homo sapiens
ALPHAILRLDLAGRD

SEQ ID NO: 41 - protein, 15 aa, Homo sapiens
SGRTTGIVMDSGDGV

SEQ ID NO: 42 - protein, 15 aa, Homo sapiens
DYLMKILTERGYSFT

SEQ ID NO: 43 - protein, 15 aa, Homo sapiens
PHAILRLDLAGRDLT

SEQ ID NO: 44 - protein, 15 aa, Homo sapiens
AMYVAIQAVLSLYAS

SEQ ID NO: 45 - protein, 15 aa, Homo sapiens
AMYVAIQAVLSLYAS

SEQ ID NO: 46 - protein, 15 aa, Homo sapiens
LPHAILRLDLAGRDL

SEQ ID NO: 47 - protein, 15 aa, Homo sapiens
TPAMYVAIQAVLSLY

SEQ ID NO: 48 - protein, 15 aa, Homo sapiens
PIYEGYALPHAILRL

SEQ ID NO: 49 - protein, 15 aa, Homo sapiens
DAPRAVFPSIVGRPR

SEQ ID NO: 50 - protein, 15 aa, Homo sapiens
STMKIKIIAPPERKY

SEQ ID NO: 51 - protein, 15 aa, Homo sapiens
GRTTGIVMDSGDGVT

SEQ ID NO: 52 - protein, 15 aa, Homo sapiens
KAGFAGDDAPRAVFP

SEQ ID NO: 53 - protein, 15 aa, Homo sapiens
LPHAILRLDLAGRDL

SEQ ID NO: 54 - protein, 15 aa, Homo sapiens
YKKLLKERKEMFSYL

SEQ ID NO: 55 - protein, 15 aa, Homo sapiens
DELRTDLKAVEAKVQ

SEQ ID NO: 56 - protein, 15 aa, Homo sapiens
NKITNSLVLDIIKLA

SEQ ID NO: 57 - protein, 14 aa, Homo sapiens
NRLDRCLKAVRKER

SEQ ID NO: 58 - protein, 12 aa, Homo sapiens
LIQQGARVGRID

SEQ ID NO: 59 - protein, 15 aa, Homo sapiens
KEMFSYLSNQIKKLS

SEQ ID NO: 60 - protein, 15 aa, Homo sapiens
GSSLLNKITNSLVLD

SEQ ID NO: 61 - protein, 15 aa, Homo sapiens
NDSFIQEISKMYPGR

SEQ ID NO: 62 - protein, 15 aa, Homo sapiens
STLELFLHELAIMDS

SEQ ID NO: 63 - protein, 15 aa, Homo sapiens
SKCMHLIQQGARVGR

SEQ ID NO: 64 - protein, 15 aa, Homo sapiens
NSLVLDIIKLAGVHT

SEQ ID NO: 65 - protein, 15 aa, Homo sapiens
RRHYRFIHGIGRSGD

SEQ ID NO: 66 - protein, 15 aa, Homo sapiens
RSHEHLIRLLLEKGK

SEQ ID NO: 67 - protein, 15 aa, Homo sapiens
SNGYKKLLKERKEMF

SEQ ID NO: 68 - protein, 15 aa, Homo sapiens
SPSLDVLITLLSLGS

SEQ ID NO: 69 - protein, 15 aa, Homo sapiens
DQKSCFKSMITAGFE

SEQ ID NO: 70 - protein, 15 aa, Homo sapiens
YTFRGFMSHTNNYPC

SEQ ID NO: 71 - protein, 15 aa, Homo sapiens
YNERLLHTPHNPISL

SEQ ID NO: 72 - protein, 15 aa, Homo sapiens
DCILCIHSTTSCFAP

SEQ ID NO: 73 - protein, 15 aa, Homo sapiens
SSLLNKITNSLVLDI

SEQ ID NO: 74 - protein, 15 aa, Homo sapiens
ALQEGLRRAVSVPLT

SEQ ID NO: 75 - protein, 15 aa, Homo sapiens
RRQFQSLDTTMRRLI

SEQ ID NO: 76 - protein, 15 aa, Homo sapiens
EQRIRLVVSRLGLDS

SEQ ID NO: 77 - protein, 15 aa, Homo sapiens
AKTQAALVLDCLETR

SEQ ID NO: 78 - protein, 15 aa, Homo sapiens
KVQGIGWYLDEKNLA

SEQ ID NO: 79 - protein, 15 aa, Homo sapiens
YFNVLINLRDITDEA

SEQ ID NO: 80 - protein, 15 aa, Homo sapiens
LPVVGSQLVGLVPLK

SEQ ID NO: 81 - protein, 15 aa, Homo sapiens
IRLVVSRLGLDSLCP

SEQ ID NO: 82 - protein, 15 aa, Homo sapiens
ELSLPVVGSQLVGLV

SEQ ID NO: 83 - protein, 15 aa, Homo sapiens
GVFGAYFNVLINLRD

SEQ ID NO: 84 - protein, 15 aa, Homo sapiens
TTMRRLIPPFREASA

SEQ ID NO: 85 - protein, 15 aa, Homo sapiens
EEEQRIRLVVSRLGL

SEQ ID NO: 86 - protein, 15 aa, Homo sapiens
GLVPLKALLDAAAFY

SEQ ID NO: 87 - protein, 15 aa, Homo sapiens
VLINLRDITDEAFKD

SEQ ID NO: 88 - protein, 15 aa, Homo sapiens
RLVVSRLGLDSLCPF

SEQ ID NO: 89 - protein, 15 aa, Homo sapiens
SNITIRNQINALTSF

SEQ ID NO: 90 - protein, 15 aa, Homo sapiens
KNNIFMSNSYPRDFV

SEQ ID NO: 91 - protein, 15 aa, Homo sapiens
LGTVLRNLKLARKLM

SEQ ID NO: 92 - protein, 15 aa, Homo sapiens
LIQPFMFRLDNRYQP

SEQ ID NO: 93 - protein, 15 aa, Homo sapiens
YLPLVLGPTAMRKYL

SEQ ID NO: 94 - protein, 15 aa, Homo sapiens
LGTVLRNLKLARKLM

SEQ ID NO: 95 - protein, 15 aa, Homo sapiens
QEARKIVGAMVQIIT

SEQ ID NO: 96 - protein, 15 aa, Homo sapiens
QQRQALAQISLPRII

SEQ ID NO: 97 - protein, 15 aa, Homo sapiens
RSLMFMQWGQLLDHD

SEQ ID NO: 98 - protein, 15 aa, Homo sapiens
RLDNRYQPMEPNPRV

SEQ ID NO: 99 - protein, 15 aa, Homo sapiens
ELLSYFKQPVAATRT

SEQ ID NO: 100 - protein, 15 aa, Homo sapiens
QLGTVLRNLKLARKL

SEQ ID NO: 101 - protein, 5 aa, Homo sapiens
SSSSS

SEQ ID NO: 102 - protein, 5 aa, Homo sapiens
DDDDD

SEQ ID NO: 103 - protein, 448 aa, Homo sapiens
MAIIYLILLFTAVRGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTESGGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGSGSGSGSC

SEQ ID NO: 104 - protein, 479 aa, Homo sapiens
MGSLQPLATLYLLGMLVASSLGGDLIAEVETDKATVGGGGGSGGGSGGSGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNSQEEKAGVVSTGLIQNGDWTFQTLVMLETVPRSGEVYTCQVEHPSMMSPLTVQWSARSESAQSKSGGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLVDKSRWQQGNVFSCSVMHEALHNHYTQKSLSLSPGK

SEQ ID NO: 105 - protein, 16 aa, Homo sapiens
LAEIETDKATIGFEVQ

SEQ ID NO: 106 - protein, 15 aa, Homo sapiens
EKPQDIEAFKNYTLD

SEQ ID NO: 107 - protein, 15 aa, Homo sapiens
VVEGALHAARTASQL

SEQ ID NO: 108 - protein, 15 aa, Homo sapiens
LITNLSSALKDETVW

SEQ ID NO: 109 - protein, 11 aa, Homo sapiens
AHHPIWARMDA

SEQ ID NO: 110 - protein, 20 aa, Homo sapiens
EVGWYRSPFSRVVHLYRNGK

(19) United States
(12) Patent Application Publication
(10) Pub. No.: US 2026/0335405 A1
(43) Pub. Date: Oct. 29, 2026

(54) METHODS OF TREATING AUTOIMMUNE DISEASE

(71) Applicant: UTI Limited Partnership, Calgary (CA)

(72) Inventor: Pedro SANTAMARIA, Calgary (CA)

(21) Appl. No.: 19/178,711

(22) Filed: Apr. 14, 2025

Related U.S. Application Data
(63) Continuation of application No. 16/767,392, filed on Nov. 28, 2018, now Pat. No. 12,397,038.
(60) Provisional application No. 62/591,921, filed on Nov. 29, 2017.
(60) Provisional application No. 62/636,520, filed on Feb. 28, 2018.
(60) Provisional application No. 62/641,607, filed on Mar. 12, 2018.

(51) Int. Cl. A61K 38/17; A61K 9/00; A61K 9/14; A61P 29/00

(52) U.S. Cl. CPC A61K 38/1774; A61K 9/0019; A61K 9/143; A61P 29/00

(57) ABSTRACT

Described herein are compositions and methods useful for treating autoimmune diseases and inflammatory disorders. The compositions and methods utilize ubiquitous, non-tissue specific antigens associated with major histocompatibility complexes (MHCs) and coupled to a nanoparticle core to induce regulatory T cells and regulatory B cells.

### CROSS REFERENCE TO RELATED APPLICATIONS

This application is a continuation of U.S. application Ser. No. 16/767,392, filed May 27, 2020, which is a national stage entry under 35 U.S.C. § 371 of International Application No. PCT/IB2018/001520, filed Nov. 28, 2018, which claims the benefit of U.S. Provisional Application No. 62/591,921 filed on Nov. 29, 2017; U.S. Provisional Application No. 62/636,520 filed on Feb. 28, 2018; and U.S. Provisional Application No. 62/641,607 filed on Mar. 12, 2018, all of which are incorporated by reference herein in their entireties.

### INCORPORATION OF SEQUENCE LISTING

The sequence listing that is contained in the file named “PTI0010-505C1-US,” which was created on Apr. 14, 2025, and which has a file size of 97,068 bytes as measured in Microsoft Windows operating system, is filed electronically herewith and incorporated herein by reference.

### SUMMARY

Provided herein are compositions comprising a plurality of antigen-major histocompatibility complexes coupled to a nanoparticle core. The compositions are useful for treating autoimmune and inflammatory disorders. Many autoimmune or inflammatory diseases are associated with an immune response directed to a particular tissue specific antigen or subset of antigens. This presents a problem for designing interventions that treat an autoimmune or inflammatory disease since each disease requires a specific medicament that targets that antigen or subset of antigens. Alternatively, nonspecific immune inhibitors can be used, but these are associated with significant systemic side-effects. Described herein are compositions comprising ubiquitous autoantigen-MHC complexes coupled to nanoparticles (uaMHC-NP), that are useful for treating autoimmune diseases or expanding populations of T regulatory cells that suppress autoreactive (i.e., autoimmune or inflammatory) T cells.

Primarily, the treatments described herein are multi-purpose in that a single composition can treat multiple-autoimmune or inflammatory disorders that are not mechanistically or pathologically linked. Many treatments, for example, corticosteroids or antibodies against general inflammatory mediators, that are multi-purpose in this way result in systemic immunosuppression, thus, increasing a treated patients risk for developing secondary infections and systemic immunological complications. The compositions described herein, while being multi-purpose, also spare systemic immunity leaving intact the ability of a patient to fight off viral, bacterial, fungal infection, or tumors. Surprisingly, the antigens utilized by the methods described herein are able to treat multiple diseases that are pathologically distinct (e.g., different autoimmune diseases of the same organ can be treated by a single antigen) and distinct with regard to tissue of origin (e.g., autoimmune diseases that afflict different organ systems). These antigens are antigens that are broadly expressed across many different tissue types, but are not the primary antigens associated with the disease, which generally display expression restricted to the particular tissue.

In certain aspects described herein is a composition comprising: (a) a plurality of antigen-major histocompatibility complexes (MHCs), each antigen-MHC of the plurality comprising a ubiquitous autoantigen, that is not a tissue specific antigen, associated with the binding groove of a MHC molecule; and (b) a nanoparticle core possessing a diameter of between 1 and 100 nanometers; wherein the antigen-MHCs are coupled to the nanoparticle core. In certain embodiments, the MHC molecule is a MHC class II molecule. In certain embodiments, the nanoparticle core is a metal or metal oxide. In certain embodiments, the metal is iron. In certain embodiments, the metal oxide is iron oxide. In certain embodiments, the diameter is between about 5 nanometers and about 50 nanometers. In certain embodiments, the diameter is between about 5 nanometers and about 25 nanometers. In certain embodiments, the plurality of antigen-MHCs is coupled to the nanoparticle core at an antigen-MHC to nanoparticle core ratio of at least 10:1. In certain embodiments, the plurality of antigen-MHCs is coupled to the nanoparticle core at an antigen-MHC to nanoparticle core ratio of no more than 150:1. In certain embodiments, the plurality of antigen-MHCs is coupled to the nanoparticle core at a density from about 0.4 to about 13 antigen-MHCs per 100 nm² of nanoparticle surface area. In certain embodiments, the antigen-MHCs are covalently coupled to the nanoparticle. In certain embodiments, the antigen-MHCs are covalently coupled to the nanoparticle core by a polyethylene glycol (PEG) linker having a mass of less than about 5 kilodaltons. In certain embodiments, the nanoparticle core further comprises a biocompatible coating. In certain embodiments, the ubiquitous autoantigen comprises a polypeptide derived from a protein that at steady-state exists in or on an intracellular compartment. In certain embodiments the intracellular compartment is cytosol, mitochondria, Golgi apparatus, endoplasmic reticulum, nucleus, or plasma membrane. In certain embodiments, the intracellular compartment is a mitochondrion. In certain embodiments, the ubiquitous autoantigen is pyruvate dehydrogenase complex-E2 component (PDC-E2). In certain embodiments, the ubiquitous autoantigen is Cytochrome P450 2D6 (CYP2D6). In certain embodiments, the ubiquitous autoantigen is soluble liver antigen (SLA). In certain embodiments, the ubiquitous autoantigen is actin (ACTB). In certain embodiments, the ubiquitous autoantigen is formimidoyltransferase-cyclodeaminase (FTCD). In certain embodiments, the ubiquitous autoantigen is myeloperoxidase (MPO). In certain embodiments, the intracellular compartment is a mitochondrion. In certain embodiments, the ubiquitous autoantigen is selected from the group consisting of: PDC-E2₃₅₃₋₃₆₇, PDC-E2₇₂₋₈₆ and PDC-E2₄₂₂₋₄₃₆ for DRB3*0202; PDC-E2₃₅₃₋₃₆₇, PDC-E2₈₀₋₉₄ and PDC-E2₅₃₅₋₅₄₉ for DRB5*0101; PDC-E2₆₂₉₋₆₄₈, PDC-E2₁₂₂₋₁₃₅ and PDC-E2₂₄₉₋₂₆₃ for DRB4*0101; and PDC-E2₂₄₉₋₂₆₃ for DRB1*0801. In certain embodiments, the ubiquitous autoantigen is selected from the group consisting of: PDC-E2₄₂₂₋₄₃₆ and PDC-E2₈₀₋₉₄. In certain embodiments, the ubiquitous autoantigen is selected from the group consisting of: CYP2D6₂₈₄₋₂₉₈, CYP2D6₂₈₉₋₃₀₃, CYP2D6₃₁₈₋₃₃₂, CYP2D6₃₁₃₋₃₃₂, CYP2D6₃₉₃₋₄₁₂, CYP2D6₁₉₂₋₂₀₆, CYP2D6₅₋₁₉, CYP2D6₂₉₃₋₃₀₇ (for DRB1*0301); CYP2D6₂₁₉₋₂₃₃, CYP2D6₂₃₇₋₂₅₁, CYP2D6₁₅₋₂₉ (for DRB3*0202); CYP2D6₂₃₅₋₂₄₉, CYP2D6₃₁₇₋₃₃₁, CYP2D6₂₉₃₋₃₀₇ (for DRB4*0101); CYP2D6₄₂₈₋₄₄₂, CYP2D6₂₃₇₋₂₅₁, CYP2D6₁₄₋₂₈ (for DRB5*0101); CYP2D6₁₉₉₋₂₁₃, CYP2D6₄₅₀₋₄₆₄, CYP2D6₃₀₁₋₃₁₅ (for DRB1*0401); CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₅₋₂₉ (for DRB1*0701). In certain embodiments, the ubiquitous autoantigen is selected from the group consisting of: ACTB₂₀₂₋₂₁₆, ACTB₁₇₀₋₁₈₄, ACTB₂₄₅₋₂₅₉, (for DRB1*0301); ACTB₁₈₇₋₂₀₁, ACTB₁₇₂₋₁₈₆, ACTB₁₃₁₋₁₄₅ (for DRB3*0202); ACTB₁₃₁₋₁₄₅, ACTB₁₇₁₋₁₈₅, ACTB₁₂₉₋₁₄₃ (for DRB4*0101); ACTB₁₆₄₋₁₇₈, ACTB₂₅₋₃₉, ACTB₃₂₃₋₃₃₇ (for DRB5*0101). In certain embodiments, the ubiquitous autoantigen is selected from the group consisting of: ACTB₁₄₆₋₁₆₀, ACTB₁₈₋₃₂, and ACTB₁₇₁₋₁₈₅. In certain embodiments, the ubiquitous autoantigen is selected from the group consisting of: SLA₃₃₄₋₃₄₈, SLA₁₉₆₋₂₁₀, SLA₁₁₅₋₁₂₉, SLA₃₇₃₋₃₈₆, SLA₁₈₆₋₁₉₇ (for DRB1*0301); SLA₃₄₂₋₂₅₆, SLA₁₁₀₋₁₂₄, SLA₂₉₉₋₃₁₃ (for DRB3*0202); SLA₄₉₋₆₃, SLA₂₆₀₋₂₇₄, SLA₁₁₉₋₁₃₃ (for DRB4*0101); SLA₈₆₋₁₀₀, SLA₂₆₋₄₀, SLA₃₃₁₋₃₄₅ (for DRB5*0101); SLA₃₁₇₋₃₃₁, SLA₁₇₁₋₁₈₅, SLA₄₁₇₋₄₃₁ (for DRB1*0401); SLA₃₅₉₋₃₇₃, SLA₂₁₅₋₂₂₉, SLA₁₁₁₋₁₂₅ (for DRB1*0701). In certain embodiments, the ubiquitous autoantigen is selected from the group consisting of: FTCD₄₃₉₋₄₅₃, FTCD₃₈₁₋₃₉₅, FTCD₂₉₇₋₃₁₁ (for DRB3*0202); FTCD₅₂₅₋₅₃₉, FTCD₂₁₈₋₂₃₂, FTCD₄₉₅₋₅₀₉ (for DRB1*0301); FTCD₂₆₂₋₂₇₆, FTCD₃₀₀₋₃₁₄, FTCD₂₅₉₋₂₇₃ (for DRB4*0101); FTCD₄₉₀₋₅₀₄, FTCD₃₈₉₋₄₀₃, FTCD₂₉₅₋₃₀₉ (for DRB5*0101). In certain embodiments, the ubiquitous autoantigen is selected from the group consisting of: FTCD₂₇₁₋₂₈₅, FTCD₄₉₈₋₅₁₂, and FTCD₃₀₁₋₃₁₅. In certain embodiments, the ubiquitous autoantigen is selected from the group consisting of: MPO₃₂₂₋₃₃₆, MPO₇₁₄₋₇₂₈, MPO₆₁₇₋₆₃₁ (for DRB3*0202); MPO₅₀₄₋₅₁₈, MPO₄₆₂₋₄₇₆, MPO₆₁₇₋₆₃₁ (for DRB1*0301); MPO₄₄₄₋₄₅₈, MPO₆₈₉₋₇₀₃, MPO₂₄₈₋₂₆₂ (for DRB4*0101); MPO₅₁₁₋₅₂₅, MPO₉₇₋₁₁₁, MPO₆₁₆₋₆₃₀ (for DRB5*0101). In certain embodiments, the complex further comprises a second plurality of antigen-major histocompatibility complexes (MHCs) coupled to the nanoparticle core, each antigen-MHC of the second plurality comprising an antigen. In certain embodiments, the antigen of the second plurality of antigen-major histocompatibility complexes (MHCs) is a second ubiquitous autoantigen. In certain embodiments, the second ubiquitous autoantigen comprises a polypeptide derived from a protein that at steady-state exists in or on an intracellular compartment. In certain embodiments, the intracellular compartment is cytosol, mitochondria, Golgi apparatus, endoplasmic reticulum, nucleus, or plasma membrane. In certain embodiments, the intracellular compartment is a mitochondrion. In certain embodiments, the intracellular compartment is a mitochondrion. In certain embodiments, the second ubiquitous autoantigen is selected from the group consisting of: PDC-E2₃₅₃₋₃₆₇, PDC-E2₇₂₋₈₆ and PDC-E2₄₂₂₋₄₃₆ for DRB3*0202; PDC-E2₃₅₃₋₃₆₇, PDC-E2₈₀₋₉₄ and PDC-E2₅₃₅₋₅₄₉ for DRB5*0101; PDC-E2₆₂₉₋₆₄₈, PDC-E2₁₂₂₋₁₃₅ and PDC-E2₂₄₉₋₂₆₃ for DRB4*0101; and PDC-E2₂₄₉₋₂₆₃ for DRB1*0801. In certain embodiments, the second ubiquitous autoantigen is selected from the group consisting of: PDC-E2₄₂₂₋₄₃₆ and PDC-E2₈₀₋₉₄. In certain embodiments, the second ubiquitous autoantigen is selected from the group consisting of: CYP2D6₂₈₄₋₂₉₈, CYP2D6₂₈₉₋₃₀₃, CYP2D6₃₁₈₋₃₃₂, CYP2D6₃₁₃₋₃₃₂, CYP2D6₃₉₃₋₄₁₂, CYP2D6₁₉₂₋₂₀₆, CYP2D6₅₋₁₉, CYP2D6₂₉₃₋₃₀₇ (for DRB1*0301); CYP2D6₂₁₉₋₂₃₃, CYP2D6₂₃₇₋₂₅₁, CYP2D6₁₅₋₂₉ (for DRB3*0202); CYP2D6₂₃₅₋₂₄₉, CYP2D6₃₁₇₋₃₃₁, CYP2D6₂₉₃₋₃₀₇ (for DRB4*0101); CYP2D6₄₂₈₋₄₄₂, CYP2D6₂₃₇₋₂₅₁, CYP2D6₁₄₋₂₈ (for DRB5*0101); CYP2D6₁₉₉₋₂₁₃, CYP2D6₄₅₀₋₄₆₄, CYP2D6₃₀₁₋₃₁₅ (for DRB1*0401); CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₅₋₂₉ (for DRB1*0701). In certain embodiments, the second ubiquitous autoantigen is selected from the group consisting of: ACTB₂₀₂₋₂₁₆, ACTB₁₇₀₋₁₈₄, ACTB₂₄₅₋₂₅₉, (for DRB1*0301); ACTB₁₈₇₋₂₀₁, ACTB₁₇₂₋₁₈₆, ACTB₁₃₁₋₁₄₅ (for DRB3*0202); ACTB₁₃₁₋₁₄₅, ACTB₁₇₁₋₁₈₅, ACTB₁₂₉₋₁₄₃ (for DRB4*0101); ACTB₁₆₄₋₁₇₈, ACTB₂₅₋₃₉, ACTB₃₂₃₋₃₃₇ (for DRB5*0101). In certain embodiments, the second ubiquitous autoantigen is selected from the group consisting of: ACTB₁₄₆₋₁₆₀, ACTB₁₈₋₃₂, and ACTB₁₇₁₋₁₈₅. In certain embodiments, the second ubiquitous autoantigen is selected from the group consisting of: SLA₃₃₄₋₃₄₈, SLA₁₉₆₋₂₁₀, SLA₁₁₅₋₁₂₉, SLA₃₇₃₋₃₈₆, SLA₁₈₆₋₁₉₇ (for DRB1*0301); SLA₃₄₂₋₂₅₆, SLA₁₁₀₋₁₂₄, SLA₂₉₉₋₃₁₃ (for DRB3*0202); SLA₄₉₋₆₃, SLA₂₆₀₋₂₇₄, SLA₁₁₉₋₁₃₃ (for DRB4*0101); SLA₈₆₋₁₀₀, SLA₂₆₋₄₀, SLA₃₃₁₋₃₄₅ (for DRB5*0101); SLA₃₁₇₋₃₃₁, SLA₁₇₁₋₁₈₅, SLA₄₁₇₋₄₃₁ (for DRB1*0401); SLA₃₅₉₋₃₇₃, SLA₂₁₅₋₂₂₉, SLA₁₁₁₋₁₂₅ (for DRB1*0701). In certain embodiments, the second ubiquitous autoantigen is selected from the group consisting of: FTCD₄₃₉₋₄₅₃, FTCD₃₈₁₋₃₉₅, FTCD₂₉₇₋₃₁₁ (for DRB3*0202); FTCD₅₂₅₋₅₃₉, FTCD₂₁₈₋₂₃₂, FTCD₄₉₅₋₅₀₉ (for DRB1*0301); FTCD₂₆₂₋₂₇₆, FTCD₃₀₀₋₃₁₄, FTCD₂₅₉₋₂₇₃ (for DRB4*0101); FTCD₄₉₀₋₅₀₄, FTCD₃₈₉₋₄₀₃, FTCD₂₉₅₋₃₀₉ (for DRB5*0101). In certain embodiments, the second ubiquitous autoantigen is selected from the group consisting of: FTCD₂₇₁₋₂₈₅, FTCD₄₉₈₋₅₁₂, and FTCD₃₀₁₋₃₁₅. In certain embodiments, the second ubiquitous autoantigen is selected from the group consisting of: MPO₃₂₂₋₃₃₆, MPO₇₁₄₋₇₂₈, MPO₆₁₇₋₆₃₁ (for DRB3*0202); MPO₅₀₄₋₅₁₈, MPO₄₆₂₋₄₇₆, MPO₆₁₇₋₆₃₁ (for DRB1*0301); MPO₄₄₄₋₄₅₈, MPO₆₈₉₋₇₀₃, MPO₂₄₈₋₂₆₂ (for DRB4*0101); MPO₅₁₁₋₅₂₅, MPO₉₇₋₁₁₁, MPO₆₁₆₋₆₃₀ (for DRB5*0101). In certain embodiments, the composition further comprises a pharmaceutically acceptable stabilizer, excipient, diluent, or combination thereof. In certain embodiments, the composition is formulated for intravenous administration. In certain embodiments, the composition is for use in a method of treating an autoimmune or inflammatory disease. In certain embodiments, the composition is for use in the manufacture of a medicament for treating an autoimmune or inflammatory disease. In certain embodiments, the autoimmune or inflammatory disease is selected from the group consisting of: type I diabetes, multiple sclerosis, relapsing-remitting multiple sclerosis, pemphigus, pemphigus foliaceus, pemphigus vulgaris, neuromyelitis optica spectrum disorder, rheumatoid arthritis, osteoarthritis, psoriatic arthritis, inflammatory bowel disease, Crohn's disease, celiac disease, allergic asthma, systemic lupus erythematosus, atherosclerosis, chronic obstructive pulmonary disease, emphysema, psoriasis, uveitis, Sjogren's Syndrome, scleroderma, anti-phospholipid syndrome, ANCA-associated vasculitis, primary biliary cirrhosis, autoimmune hepatitis, primary sclerosing cholangitis, and Stiff Man Syndrome. In certain embodiments, the autoimmune or inflammatory disease is multiple sclerosis. In certain embodiments, the ubiquitous autoantigen is not a polypeptide derived from myelin basic protein, myelin associated glycoprotein, myelin oligodendrocyte protein (MOG), proteolipid protein, oligodendrocyte myelin oligoprotein, myelin associated oligodendrocyte basic protein, oligodendrocyte specific protein, heat shock proteins, an oligodendrocyte specific protein, NOGO A, glycoprotein Po, peripheral myelin protein 22, or 2′3′-cyclic nucleotide 3′-phosphodiesterase. In certain embodiments, the autoimmune or inflammatory disease is type I diabetes. In certain embodiments, the ubiquitous autoantigen is a polypeptide derived from pre-proinsulin, proinsulin, islet-specific glucose-6-phosphatase (IGRP), glutamate decarboxylase (GAD), islet cell autoantigen-2 (ICA2), or insulin.

In certain aspects described herein is a method of treating an autoimmune or inflammatory disease comprising administering to an individual a therapeutically effective amount of a composition comprising: (a) a plurality of antigen-major histocompatibility complexes (MHCs), each antigen-MHC of the plurality comprising a ubiquitous autoantigen, that is not a tissue specific antigen, associated with the binding groove of an MHC molecule; and (b) a nanoparticle core possessing a diameter of between 1 and 100 nanometers; wherein the antigen-MHCs are coupled to the nanoparticle core. In certain embodiments, the MHC molecule is a MHC class II molecule. In certain embodiments, the nanoparticle core is a metal or metal oxide. In certain embodiments, the metal is iron. In certain embodiments, the metal oxide is iron oxide. In certain embodiments, the diameter is between about 5 nanometers and about 50 nanometers. In certain embodiments, the diameter is between about 5 nanometers and about 25 nanometers. In certain embodiments, the plurality of antigen-MHCs is coupled to the nanoparticle core at an antigen-MHC to nanoparticle core ratio of at least 10:1. In certain embodiments, the plurality of antigen-MHCs is coupled to the nanoparticle core at an antigen-MHC to nanoparticle core ratio of no more than 150:1. In certain embodiments, the plurality of antigen-MHCs is coupled to the nanoparticle core at a density from about 0.4 to about 13 antigen-MHCs per 100 nm² of nanoparticle core surface area. In certain embodiments, the antigen-MHCs are covalently coupled to the nanoparticle core. In certain embodiments, the antigen-MHCs are covalently coupled to the nanoparticle core by a polyethylene glycol (PEG) linker having a mass of less than about 5 kilodaltons. In certain embodiments, the nanoparticle core further comprises a biocompatible coating. In certain embodiments, the ubiquitous autoantigen comprises a polypeptide derived from a protein that at steady-state exists in or on an intracellular compartment. In certain embodiments, the intracellular compartment is cytosol, mitochondria, Golgi apparatus, endoplasmic reticulum, nucleus, or plasma membrane. In certain embodiments, the intracellular compartment is a mitochondrion. In certain embodiments, the ubiquitous autoantigen is pyruvate dehydrogenase complex-E2 component (PDC-E2). In certain embodiments, the ubiquitous autoantigen is Cytochrome P450 2D6 (CYP2D6). In certain embodiments, the ubiquitous autoantigen is soluble liver antigen (SLA). In certain embodiments, the ubiquitous autoantigen is actin (ACTB). In certain embodiments, the ubiquitous autoantigen is formimidoyltransferase-cyclodeaminase (FTCD). In certain embodiments, the ubiquitous autoantigen is myeloperoxidase (MPO). In certain embodiments, the intracellular compartment is a mitochondrion. In certain embodiments, the ubiquitous autoantigen is selected from the group consisting of: PDC-E2₃₅₃₋₃₆₇, PDC-E2₇₂₋₈₆ and PDC-E2₄₂₂₋₄₃₆ for DRB3*0202; PDC-E2₃₅₃₋₃₆₇, PDC-E2₈₀₋₉₄ and PDC-E2₅₃₅₋₅₄₉ for DRB5*0101; PDC-E2₆₂₉₋₆₄₈, PDC-E2₁₂₂₋₁₃₅ and PDC-E2₂₄₉₋₂₆₃ for DRB4*0101; and PDC-E2₂₄₉₋₂₆₃ for DRB1*0801. In certain embodiments, the ubiquitous autoantigen is selected from the group consisting of: PDC-E2₄₂₂₋₄₃₆ and PDC-E2₈₀₋₉₄. In certain embodiments, the ubiquitous autoantigen is selected from the group consisting of: CYP2D6₂₈₄₋₂₉₈, CYP2D6₂₈₉₋₃₀₃, CYP2D6₃₁₈₋₃₃₂, CYP2D6₃₁₃₋₃₃₂, CYP2D6₃₉₃₋₄₁₂, CYP2D6₁₉₂₋₂₀₆, CYP2D6₅₋₁₉, CYP2D6₂₉₃₋₃₀₇ (for DRB1*0301); CYP2D6₂₁₉₋₂₃₃, CYP2D6₂₃₇₋₂₅₁, CYP2D6₁₅₋₂₉ (for DRB3*0202); CYP2D6₂₃₅₋₂₄₉, CYP2D6₃₁₇₋₃₃₁, CYP2D6₂₉₃₋₃₀₇ (for DRB4*0101); CYP2D6₄₂₈₋₄₄₂, CYP2D6₂₃₇₋₂₅₁, CYP2D6₁₄₋₂₈ (for DRB5*0101); CYP2D6₁₉₉₋₂₁₃, CYP2D6₄₅₀₋₄₆₄, CYP2D6₃₀₁₋₃₁₅ (for DRB1*0401); CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₅₋₂₉ (for DRB1*0701). In certain embodiments, the ubiquitous autoantigen is selected from the group consisting of: ACTB₂₀₂₋₂₁₆, ACTB₁₇₀₋₁₈₄, ACTB₂₄₅₋₂₅₉, (for DRB1*0301); ACTB₁₈₇₋₂₀₁, ACTB₁₇₂₋₁₈₆, ACTB₁₃₁₋₁₄₅ (for DRB3*0202); ACTB₁₃₁₋₁₄₅, ACTB₁₇₁₋₁₈₅, ACTB₁₂₉₋₁₄₃ (for DRB4*0101); ACTB₁₆₄₋₁₇₈, ACTB₂₅₋₃₉, ACTB₃₂₃₋₃₃₇ (for DRB5*0101). In certain embodiments, the ubiquitous autoantigen is selected from the group consisting of: ACTB₁₄₆₋₁₆₀, ACTB₁₈₋₃₂, and ACTB₁₇₁₋₁₈₅. In certain embodiments, the ubiquitous autoantigen is selected from the group consisting of: SLA₃₃₄₋₃₄₈, SLA₁₉₆₋₂₁₀, SLA₁₁₅₋₁₂₉, SLA₃₇₃₋₃₈₆, SLA₁₈₆₋₁₉₇ (for DRB1*0301); SLA₃₄₂₋₂₅₆, SLA₁₁₀₋₁₂₄, SLA₂₉₉₋₃₁₃ (for DRB3*0202); SLA₄₉₋₆₃, SLA₂₆₀₋₂₇₄, SLA₁₁₉₋₁₃₃ (for DRB4*0101); SLA₈₆₋₁₀₀, SLA₂₆₋₄₀, SLA₃₃₁₋₃₄₅ (for DRB5*0101); SLA₃₁₇₋₃₃₁, SLA₁₇₁₋₁₈₅, SLA₄₁₇₋₄₃₁ (for DRB1*0401); SLA₃₅₉₋₃₇₃, SLA₂₁₅₋₂₂₉, SLA₁₁₁₋₁₂₅ (for DRB1*0701). In certain embodiments, the ubiquitous autoantigen is selected from the group consisting of: FTCD₄₃₉₋₄₅₃, FTCD₃₈₁₋₃₉₅, FTCD₂₉₇₋₃₁₁ (for DRB3*0202); FTCD₅₂₅₋₅₃₉, FTCD₂₁₈₋₂₃₂, FTCD₄₉₅₋₅₀₉ (for DRB1*0301); FTCD₂₆₂₋₂₇₆, FTCD₃₀₀₋₃₁₄, FTCD₂₅₉₋₂₇₃ (for DRB4*0101); FTCD₄₉₀₋₅₀₄, FTCD₃₈₉₋₄₀₃, FTCD₂₉₅₋₃₀₉ (for DRB5*0101). In certain embodiments, the ubiquitous autoantigen is selected from the group consisting of: FTCD₂₇₁₋₂₈₅, FTCD₄₉₈₋₅₁₂, and FTCD₃₀₁₋₃₁₅. In certain embodiments, the ubiquitous autoantigen is selected from the group consisting of: MPO₃₂₂₋₃₃₆, MPO₇₁₄₋₇₂₈, MPO₆₁₇₋₆₃₁ (for DRB3*0202); MPO₅₀₄₋₅₁₈, MPO₄₆₂₋₄₇₆, MPO₆₁₇₋₆₃₁ (for DRB1*0301); MPO₄₄₄₋₄₅₈, MPO₆₈₉₋₇₀₃, MPO₂₄₈₋₂₆₂ (for DRB4*0101); MPO₅₁₁₋₅₂₅, MPO₉₇₋₁₁₁, MPO₆₁₆₋₆₃₀ (for DRB5*0101). In certain embodiments, the complex further comprises a second plurality of antigen-major histocompatibility complexes (MHCs) coupled to the nanoparticle core, each antigen-MHC of the second plurality comprising an antigen. In certain embodiments, the antigen of the second plurality of antigen-major histocompatibility complexes (MHCs) is a second ubiquitous autoantigen. In certain embodiments, the second ubiquitous autoantigen comprises a polypeptide derived from a protein that at steady-state exists in or on an intracellular compartment. In certain embodiments, the intracellular compartment is cytosol, mitochondria, Golgi apparatus, endoplasmic reticulum, nucleus, or plasma membrane. In certain embodiments, the intracellular compartment is a mitochondrion. In certain embodiments, the intracellular compartment is a mitochondrion. In certain embodiments, the second ubiquitous autoantigen is selected from the group consisting of: PDC-E2₃₅₃₋₃₆₇, PDC-E2₇₂₋₈₆ and PDC-E2₄₂₂₋₄₃₆ for DRB3*0202; PDC-E2₃₅₃₋₃₆₇, PDC-E2₈₀₋₉₄ and PDC-E2₅₃₅₋₅₄₉ for DRB5*0101; PDC-E2₆₂₉₋₆₄₈, PDC-E2₁₂₂₋₁₃₅ and PDC-E2₂₄₉₋₂₆₃ for DRB4*0101; and PDC-E2₂₄₉₋₂₆₃ for DRB1*0801. In certain embodiments, the second ubiquitous autoantigen is selected from the group consisting of: PDC-E2₄₂₂₋₄₃₆ and PDC-E2₈₀₋₉₄. In certain embodiments, the second ubiquitous autoantigen is selected from the group consisting of: CYP2D6₂₈₄₋₂₉₈, CYP2D6₂₈₉₋₃₀₃, CYP2D6₃₁₈₋₃₃₂, CYP2D6₃₁₃₋₃₃₂, CYP2D6₃₉₃₋₄₁₂, CYP2D6₁₉₂₋₂₀₆, CYP2D6₅₋₁₉, CYP2D6₂₉₃₋₃₀₇ (for DRB1*0301); CYP2D6₂₁₉₋₂₃₃, CYP2D6₂₃₇₋₂₅₁, CYP2D6₁₅₋₂₉ (for DRB3*0202); CYP2D6₂₃₅₋₂₄₉, CYP2D6₃₁₇₋₃₃₁, CYP2D6₂₉₃₋₃₀₇ (for DRB4*0101); CYP2D6₄₂₈₋₄₄₂, CYP2D6₂₃₇₋₂₅₁, CYP2D6₁₄₋₂₈ (for DRB5*0101); CYP2D6₁₉₉₋₂₁₃, CYP2D6₄₅₀₋₄₆₄, CYP2D6₃₀₁₋₃₁₅ (for DRB1*0401); CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₅₋₂₉ (for DRB1*0701). In certain embodiments, the second ubiquitous autoantigen is selected from the group consisting of: ACTB₂₀₂₋₂₁₆, ACTB₁₇₀₋₁₈₄, ACTB₂₄₅₋₂₅₉, (for DRB1*0301); ACTB₁₈₇₋₂₀₁, ACTB₁₇₂₋₁₈₆, ACTB₁₃₁₋₁₄₅ (for DRB3*0202); ACTB₁₃₁₋₁₄₅, ACTB₁₇₁₋₁₈₅, ACTB₁₂₉₋₁₄₃ (for DRB4*0101); ACTB₁₆₄₋₁₇₈, ACTB₂₅₋₃₉, ACTB₃₂₃₋₃₃₇ (for DRB5*0101). In certain embodiments, the second ubiquitous autoantigen is selected from the group consisting of: ACTB₁₄₆₋₁₆₀, ACTB₁₈₋₃₂, and ACTB₁₇₁₋₁₈₅. In certain embodiments, the second ubiquitous autoantigen is selected from the group consisting of: SLA₃₃₄₋₃₄₈, SLA₁₉₆₋₂₁₀, SLA₁₁₅₋₁₂₉, SLA₃₇₃₋₃₈₆, SLA₁₈₆₋₁₉₇ (for DRB1*0301); SLA 342-256, SLA₁₁₀₋₁₂₄, SLA₂₉₉₋₃₁₃ (for DRB3*0202); SLA₄₉₋₆₃, SLA₂₆₀₋₂₇₄, SLA₁₁₉₋₁₃₃ (for DRB4*0101); SLA₈₆₋₁₀₀, SLA₂₆₋₄₀, SLA₃₃₁₋₃₄₅ (for DRB5*0101); SLA₃₁₇₋₃₃₁, SLA₁₇₁₋₁₈₅, SLA₄₁₇₋₄₃₁ (for DRB1*0401); SLA₃₅₉₋₃₇₃, SLA₂₁₅₋₂₂₉, SLA₁₁₁₋₁₂₅ (for DRB1*0701). In certain embodiments, the second ubiquitous autoantigen is selected from the group consisting of: FTCD₄₃₉₋₄₅₃, FTCD₃₈₁₋₃₉₅, FTCD₂₉₇₋₃₁₁ (for DRB3*0202); FTCD₅₂₅₋₅₃₉, FTCD₂₁₈₋₂₃₂, FTCD₄₉₅₋₅₀₉ (for DRB1*0301); FTCD₂₆₂₋₂₇₆, FTCD₃₀₀₋₃₁₄, FTCD₂₅₉₋₂₇₃ (for DRB4*0101); FTCD₄₉₀₋₅₀₄, FTCD₃₈₉₋₄₀₃, FTCD₂₉₅₋₃₀₉ (for DRB5*0101). In certain embodiments, the second ubiquitous autoantigen is selected from the group consisting of: FTCD₂₇₁₋₂₈₅, FTCD₄₉₈₋₅₁₂, and FTCD₃₀₁₋₃₁₅. In certain embodiments, the second ubiquitous autoantigen is selected from the group consisting of: MPO₃₂₂₋₃₃₆, MPO₇₁₄₋₇₂₈, MPO₆₁₇₋₆₃₁ (for DRB3*0202); MPO₅₀₄₋₅₁₈, MPO₄₆₂₋₄₇₆, MPO₆₁₇₋₆₃₁ (for DRB1*0301); MPO₄₄₄₋₄₅₈, MPO₆₈₉₋₇₀₃, MPO₂₄₈₋₂₆₂ (for DRB4*0101); MPO₅₁₁₋₅₂₅, MPO₉₇₋₁₁₁, MPO₆₁₆₋₆₃₀ (for DRB5*0101). In certain embodiments, the composition further comprises a pharmaceutically acceptable stabilizer, excipient, diluent, or combination thereof. In certain embodiments, the composition is formulated for intravenous administration. In certain embodiments, the autoimmune or inflammatory disease is selected from the group consisting of type I diabetes, multiple sclerosis, relapsing-remitting multiple sclerosis, pemphigus, pemphigus foliaceus, pemphigus vulgaris, neuromyelitis optica spectrum disorder, rheumatoid arthritis, osteoarthritis, psoriatic arthritis, inflammatory bowel disease, Crohn's disease, celiac disease, allergic asthma, systemic lupus erythematosus, atherosclerosis, chronic obstructive pulmonary disease, emphysema, psoriasis, uveitis, Sjogren's Syndrome, scleroderma, anti-phospholipid syndrome, ANCA-associated vasculitis, primary biliary cirrhosis, autoimmune hepatitis, primary sclerosing cholangitis, and Stiff Man Syndrome. In certain embodiments, the autoimmune or inflammatory disease is multiple sclerosis. In certain embodiments, the ubiquitous autoantigen is not a polypeptide derived from myelin basic protein, myelin associated glycoprotein, myelin oligodendrocyte protein (MOG), proteolipid protein, oligodendrocyte myelin oligoprotein, myelin associated oligodendrocyte basic protein, oligodendrocyte specific protein, heat shock proteins, an oligodendrocyte specific protein, NOGO A, glycoprotein Po, peripheral myelin protein 22, or 2′3′-cyclic nucleotide 3′-phosphodiesterase. In certain embodiments, the autoimmune or inflammatory disease is type I diabetes. In certain embodiments, the ubiquitous autoantigen is a polypeptide derived from pre-proinsulin, proinsulin, islet-specific glucose-6-phosphatase (IGRP), glutamate decarboxylase (GAD), islet cell autoantigen-2 (ICA2), or insulin.

In another aspect described herein is a method of treating multiple sclerosis comprising administering to an individual a therapeutically effective amount of a composition comprising: (a) a plurality of antigen-major histocompatibility complexes (MHCs), each antigen-MHC of the plurality comprising a ubiquitous autoantigen, which is not a multiple sclerosis specific antigen, associated with the binding groove of an MHC molecule; and (a) a nanoparticle core, the nanoparticle core possessing a diameter of between 1 and 100 nanometers; wherein the antigen-MHCs are coupled to the nanoparticle core. In certain embodiments, the MHC molecule is a MHC class II molecule. In certain embodiments, the nanoparticle core is a metal or metal oxide. In certain embodiments, the metal is iron. In certain embodiments, the metal oxide is iron oxide. In certain embodiments, the diameter is between about 5 nanometers and about 50 nanometers. In certain embodiments, the diameter is between about 5 nanometers and about 25 nanometers. In certain embodiments, the plurality of antigen-MHCs is coupled to the nanoparticle core at an antigen-MHC to nanoparticle core ratio of at least 10:1. In certain embodiments, the plurality of antigen-MHCs is coupled to the nanoparticle core at an antigen-MHC to nanoparticle core ratio of no more than 150:1. In certain embodiments, the plurality of antigen-MHCs is coupled to the nanoparticle core at a density from about 0.4 to about 13 antigen-MHCs per 100 nm² of nanoparticle core surface area. In certain embodiments, the antigen-MHCs are covalently coupled to the nanoparticle core. In certain embodiments, the antigen-MHCs are covalently coupled to the nanoparticle core by a polyethylene glycol (PEG) linker having a mass of less than about 5 kilodaltons. In certain embodiments, the nanoparticle core further comprises a biocompatible coating. In certain embodiments, the ubiquitous autoantigen comprises a polypeptide derived from a protein that at steady-state exists in or on an intracellular compartment. In certain embodiments, the intracellular compartment is cytosol. In certain embodiments, the intracellular compartment is a mitochondrion. In certain embodiments, the ubiquitous autoantigen is pyruvate dehydrogenase complex-E2 component (PDC-E2). In certain embodiments, the ubiquitous autoantigen is Cytochrome P450 2D6 (CYP2D6). In certain embodiments, the ubiquitous autoantigen is soluble liver antigen (SLA). In certain embodiments, the ubiquitous autoantigen is actin (ACTB). In certain embodiments, the ubiquitous autoantigen is formimidoyltransferase-cyclodeaminase (FTCD). In certain embodiments, the ubiquitous autoantigen is myeloperoxidase (MPO). In certain embodiments, the intracellular compartment is a mitochondrion. In certain embodiments, the ubiquitous autoantigen is selected from the group consisting of: PDC-E2₃₅₃₋₃₆₇, PDC-E2₇₂₋₈₆ and PDC-E2₄₂₂₋₄₃₆ for DRB3*0202; PDC-E2₃₅₃₋₃₆₇, PDC-E2₈₀₋₉₄ and PDC-E2₅₃₅₋₅₄₉ for DRB5*0101; PDC-E2₆₂₉₋₆₄₈, PDC-E2₁₂₂₋₁₃₅ and PDC-E2249-263 for DRB4*0101; and PDC-E2₂₄₉₋₂₆₃ for DRB1*0801. In certain embodiments, the ubiquitous autoantigen is selected from the group consisting of: PDC-E2₄₂₂₋₄₃₆ and PDC-E2₈₀₋₉₄. In certain embodiments, the ubiquitous autoantigen is selected from the group consisting of: CYP2D6₂₈₄₋₂₉₈, CYP2D6₂₈₉₋₃₀₃, CYP2D6₃₁₈₋₃₃₂, CYP2D6₃₁₃₋₃₃₂, CYP2D6₃₉₃₋₄₁₂, CYP2D6₁₉₂₋₂₀₆, CYP2D6₅₋₁₉, CYP2D6₂₉₃₋₃₀₇ (for DRB1*0301); CYP2D6₂₁₉₋₂₃₃, CYP2D6₂₃₇₋₂₅₁, CYP2D6₁₅₋₂₉ (for DRB3*0202); CYP2D6₂₃₅₋₂₄₉, CYP2D6₃₁₇₋₃₃₁, CYP2D6₂₉₃₋₃₀₇ (for DRB4*0101); CYP2D6₄₂₈₋₄₄₂, CYP2D6₂₃₇₋₂₅₁, CYP2D6₁₄₋₂₈ (for DRB5*0101); CYP2D6₁₉₉₋₂₁₃, CYP2D6₄₅₀₋₄₆₄, CYP2D6₃₀₁₋₃₁₅ (for DRB1*0401); CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₅₋₂₉ (for DRB1*0701). In certain embodiments, the ubiquitous autoantigen is selected from the group consisting of: ACTB₂₀₂₋₂₁₆, ACTB₁₇₀₋₁₈₄, ACTB₂₄₅₋₂₅₉, (for DRB1*0301); ACTB₁₈₇₋₂₀₁, ACTB₁₇₂₋₁₈₆, ACTB₁₃₁₋₁₄₅ (for DRB3*0202); ACTB₁₃₁₋₁₄₅, ACTB₁₇₁₋₁₈₅, ACTB₁₂₉₋₁₄₃ (for DRB4*0101); ACTB₁₆₄₋₁₇₈, ACTB₂₅₋₃₉, ACTB₃₂₃₋₃₃₇ (for DRB5*0101). In certain embodiments, the ubiquitous autoantigen is selected from the group consisting of: ACTB₁₄₆₋₁₆₀, ACTB₁₈₋₃₂, and ACTB₁₇₁₋₁₈₅. In certain embodiments, the ubiquitous autoantigen is selected from the group consisting of: SLA₃₃₄₋₃₄₈, SLA₁₉₆₋₂₁₀, SLA₁₁₅₋₁₂₉, SLA₃₇₃₋₃₈₆, SLA₁₈₆₋₁₉₇ (for DRB1*0301); SLA₃₄₂₋₂₅₆, SLA₁₁₀₋₁₂₄, SLA₂₉₉₋₃₁₃ (for DRB3*0202); SLA₄₉₋₆₃, SLA₂₆₀₋₂₇₄, SLA₁₁₉₋₁₃₃ (for DRB4*0101); SLA₈₆₋₁₀₀, SLA₂₆₋₄₀, SLA₃₃₁₋₃₄₅ (for DRB5*0101); SLA₃₁₇₋₃₃₁, SLA₁₇₁₋₁₈₅, SLA₄₁₇₋₄₃₁ (for DRB1*0401); SLA₃₅₉₋₃₇₃, SLA₂₁₅₋₂₂₉, SLA₁₁₁₋₁₂₅ (for DRB1*0701). In certain embodiments, the ubiquitous autoantigen is selected from the group consisting of: FTCD₄₃₉₋₄₅₃, FTCD₃₈₁₋₃₉₅, FTCD₂₉₇₋₃₁₁ (for DRB3*0202); FTCD₅₂₅₋₅₃₉, FTCD₂₁₈₋₂₃₂, FTCD₄₉₅₋₅₀₉ (for DRB1*0301); FTCD₂₆₂₋₂₇₆, FTCD₃₀₀₋₃₁₄, FTCD₂₅₉₋₂₇₃ (for DRB4*0101); FTCD₄₉₀₋₅₀₄, FTCD₃₈₉₋₄₀₃, FTCD₂₉₅₋₃₀₉ (for DRB5*0101). In certain embodiments, the ubiquitous autoantigen is selected from the group consisting of: FTCD₂₇₁₋₂₈₅, FTCD₄₉₈₋₅₁₂, and FTCD₃₀₁₋₃₁₅. In certain embodiments, the ubiquitous autoantigen is selected from the group consisting of: MPO₃₂₂₋₃₃₆, MPO₇₁₄₋₇₂₈, MPO₆₁₇₋₆₃₁ (for DRB3*0202); MPO₅₀₄₋₅₁₈, MPO₄₆₂₋₄₇₆, MPO₆₁₇₋₆₃₁ (for DRB1*0301); MPO₄₄₄₋₄₅₈, MPO₆₈₉₋₇₀₃, MPO₂₄₈₋₂₆₂ (for DRB4*0101); MPO₅₁₁₋₅₂₅, MPO₉₇₋₁₁₁, MPO₆₁₆₋₆₃₀ (for DRB5*0101). In certain embodiments, the complex further comprises a second plurality of antigen-major histocompatibility complexes (MHCs) coupled to the nanoparticle core, each antigen-MHC of the second plurality comprising an antigen. In certain embodiments, the antigen of the second plurality of antigen-major histocompatibility complexes (MHCs) is a second ubiquitous autoantigen. In certain embodiments, the second ubiquitous autoantigen comprises a polypeptide derived from a protein that at steady-state exists in or on an intracellular compartment. In certain embodiments, the intracellular compartment is cytosol. In certain embodiments, the intracellular compartment is a mitochondrion. In certain embodiments, the second ubiquitous autoantigen is selected from the group consisting of: PDC-E2₃₅₃₋₃₆₇, PDC-E2₇₂₋₈₆ and PDC-E2₄₂₂₋₄₃₆ for DRB3*0202; PDC-E2₃₅₃₋₃₆₇, PDC-E2₈₀₋₉₄ and PDC-E2₅₃₅₋₅₄₉ for DRB5*0101; PDC-E2₆₂₉₋₆₄₈, PDC-E2₁₂₂₋₁₃₅ and PDC-E2₂₄₉₋₂₆₃ for DRB4*0101; and PDC-E2₂₄₉₋₂₆₃ for DRB1*0801. In certain embodiments, the second ubiquitous autoantigen is selected from the group consisting of: PDC-E2₄₂₂₋₄₃₆ and PDC-E2₈₀₋₉₄. In certain embodiments, the second ubiquitous autoantigen is selected from the group consisting of: CYP2D6₂₈₄₋₂₉₈, CYP2D6₂₈₉₋₃₀₃, CYP2D6₃₁₈₋₃₃₂, CYP2D6₃₁₃₋₃₃₂, CYP2D6₃₉₃₋₄₁₂, CYP2D6₁₉₂₋₂₀₆, CYP2D6₅₋₁₉, CYP2D6₂₉₃₋₃₀₇ (for DRB1*0301); CYP2D6₂₁₉₋₂₃₃, CYP2D6₂₃₇₋₂₅₁, CYP2D6₁₅₋₂₉ (for DRB3*0202); CYP2D6₂₃₅₋₂₄₉, CYP2D6₃₁₇₋₃₃₁, CYP2D6₂₉₃₋₃₀₇ (for DRB4*0101); CYP2D6₄₂₈₋₄₄₂, CYP2D6₂₃₇₋₂₅₁, CYP2D6₁₄₋₂₈ (for DRB5*0101); CYP2D6₁₉₉₋₂₁₃, CYP2D6₄₅₀₋₄₆₄, CYP2D6₃₀₁₋₃₁₅ (for DRB1*0401); CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₅₋₂₉ (for DRB1*0701). In certain embodiments, the second ubiquitous autoantigen is selected from the group consisting of: ACTB₂₀₂₋₂₁₆, ACTB₁₇₀₋₁₈₄, ACTB₂₄₅₋₂₅₉, (for DRB1*0301); ACTB₁₈₇₋₂₀₁, ACTB₁₇₂₋₁₈₆, ACTB₁₃₁₋₁₄₅ (for DRB3*0202); ACTB₁₃₁₋₁₄₅, ACTB₁₇₁₋₁₈₅, ACTB₁₂₉₋₁₄₃ (for DRB4*0101); ACTB₁₆₄₋₁₇₈, ACTB₂₅₋₃₉, ACTB₃₂₃₋₃₃₇ (for DRB5*0101). In certain embodiments, the second ubiquitous autoantigen is selected from the group consisting of: ACTB₁₄₆₋₁₆₀, ACTB₁₈₋₃₂, and ACTB₁₇₁₋₁₈₅. In certain embodiments, the ubiquitous autoantigen is selected from the group consisting of: SLA₃₃₄₋₃₄₈, SLA 196-210, SLA₁₁₅₋₁₂₉, SLA₃₇₃₋₃₈₆, SLA₁₈₆₋₁₉₇ (for DRB1*0301); SLA₃₄₂₋₂₅₆, SLA₁₁₀₋₁₂₄, SLA₂₉₉₋₃₁₃ (for DRB3*0202); SLA₄₉₋₆₃, SLA₂₆₀₋₂₇₄, SLA₁₁₉₋₁₃₃ (for DRB4*0101); SLA₈₆₋₁₀₀, SLA₂₆₋₄₀, SLA₃₃₁₋₃₄₅ (for DRB5*0101); SLA₃₁₇₋₃₃₁, SLA₁₇₁₋₁₈₅, SLA₄₁₇₋₄₃₁ (for DRB1*0401); SLA₃₅₉₋₃₇₃, SLA₂₁₅₋₂₂₉, SLA₁₁₁₋₁₂₅ (for DRB1*0701). In certain embodiments, the second ubiquitous autoantigen is selected from the group consisting of: FTCD 439-453, FTCD₃₈₁₋₃₉₅, FTCD₂₉₇₋₃₁₁ (for DRB3*0202); FTCD₅₂₅₋₅₃₉, FTCD₂₁₈₋₂₃₂, FTCD₄₉₅₋₅₀₉ (for DRB1*0301); FTCD₂₆₂₋₂₇₆, FTCD₃₀₀₋₃₁₄, FTCD₂₅₉₋₂₇₃ (for DRB4*0101); FTCD₄₉₀₋₅₀₄, FTCD₃₈₉₋₄₀₃, FTCD₂₉₅₋₃₀₉ (for DRB5*0101). In certain embodiments, the second ubiquitous autoantigen is selected from the group consisting of: FTCD₂₇₁₋₂₈₅, FTCD₄₉₈₋₅₁₂, and FTCD₃₀₁₋₃₁₅. In certain embodiments, the ubiquitous autoantigen is selected from the group consisting of: MPO 322-336, MPO₇₁₄₋₇₂₈, MPO₆₁₇₋₆₃₁ (for DRB3*0202); MPO₅₀₄₋₅₁₈, MPO₄₆₂₋₄₇₆, MPO₆₁₇₋₆₃₁ (for DRB1*0301); MPO₄₄₄₋₄₅₈, MPO₆₈₉₋₇₀₃, MPO₂₄₈₋₂₆₂ (for DRB4*0101); MPO₅₁₁₋₅₂₅, MPO₉₇₋₁₁₁, MPO₆₁₆₋₆₃₀ (for DRB5*0101). In certain embodiments, the composition further comprises a pharmaceutically acceptable stabilizer, excipient, diluent, or combination thereof. In certain embodiments, the composition is formulated for intravenous administration. In certain embodiments, the ubiquitous autoantigen is not a polypeptide derived from myelin basic protein, myelin associated glycoprotein, myelin oligodendrocyte protein (MOG), proteolipid protein, oligodendrocyte myelin oligoprotein, myelin associated oligodendrocyte basic protein, oligodendrocyte specific protein, heat shock proteins, an oligodendrocyte specific protein, NOGO A, glycoprotein Po, peripheral myelin protein 22, or 2′3′-cyclic nucleotide 3′-phosphodiesterase.

In another aspect described herein is a method of treating type I diabetes comprising administering to an individual a therapeutically effective amount of a composition comprising: (a) a plurality of antigen-major histocompatibility complexes (MHCs), each antigen-MHC of the plurality comprising a ubiquitous autoantigen, which is not a type I diabetes specific antigen, associated with the binding groove of an MHC molecule; and (b) a nanoparticle core, the nanoparticle core possessing a diameter of between 1 and 100 nanometers; wherein the plurality of antigen-MHCs is coupled to the nanoparticle core. In certain embodiments, the MHC molecule is a MHC class II molecule. In certain embodiments, the nanoparticle core is a metal or metal oxide. In certain embodiments, the metal is iron. In certain embodiments, the metal oxide is iron oxide. In certain embodiments, the diameter is between about 5 nanometers and about 50 nanometers. In certain embodiments, the diameter is between about 5 nanometers and about 25 nanometers. In certain embodiments, the plurality of antigen-MHCs is coupled to the nanoparticle core at an antigen-MHC to nanoparticle core ratio of at least 10:1. In certain embodiments, the plurality of antigen-MHCs is coupled to the nanoparticle core at an antigen-MHC to nanoparticle core ratio of no more than 150:1. In certain embodiments, the plurality of antigen-MHCs is coupled to the nanoparticle at a density from about 0.4 to about 13 antigen-MHCs per 100 nm² of nanoparticle surface area. In certain embodiments, the antigen-MHCs are covalently coupled to the nanoparticle core. In certain embodiments, the antigen-MHCs are covalently coupled to the nanoparticle core by a polyethylene glycol (PEG) linker having a mass of less than about 5 kilodaltons. In certain embodiments, the nanoparticle core further comprises a biocompatible coating. In certain embodiments, the ubiquitous autoantigen comprises a polypeptide derived from a protein that at steady-state exists in or on an intracellular compartment. In certain embodiments, the intracellular compartment is cytosol. In certain embodiments, the intracellular compartment is a mitochondrion. In certain embodiments, the ubiquitous autoantigen is pyruvate dehydrogenase complex-E2 component (PDC-E2). In certain embodiments, the ubiquitous autoantigen is Cytochrome P450 2D6 (CYP2D6). In certain embodiments, the ubiquitous autoantigen is soluble liver antigen (SLA). In certain embodiments, the ubiquitous autoantigen is actin (ACTB). In certain embodiments, the ubiquitous autoantigen is formimidoyltransferase-cyclodeaminase (FTCD). In certain embodiments, the ubiquitous autoantigen is myeloperoxidase (MPO). In certain embodiments, the intracellular compartment is a mitochondrion. In certain embodiments, the ubiquitous autoantigen is selected from the group consisting of: PDC-E2₃₅₃₋₃₆₇, PDC-E2₇₂₋₈₆ and PDC-E2₄₂₂₋₄₃₆ for DRB3*0202; PDC-E2₃₅₃₋₃₆₇, PDC-E2₈₀₋₉₄ and PDC-E2₅₃₅₋₅₄₉ for DRB5*0101; PDC-E2₆₂₉₋₆₄₈, PDC-E2₁₂₂₋₁₃₅ and PDC-E2₂₄₉₋₂₆₃ for DRB4*0101; and PDC-E2₂₄₉₋₂₆₃ for DRB1*0801. In certain embodiments, the ubiquitous autoantigen is selected from the group consisting of: PDC-E2₄₂₂₋₄₃₆ and PDC-E2₈₀₋₉₄. In certain embodiments, the ubiquitous autoantigen is selected from the group consisting of: CYP2D6₂₈₄₋₂₉₈, CYP2D6₂₈₉₋₃₀₃, CYP2D6₃₁₈₋₃₃₂, CYP2D6₃₁₃₋₃₃₂, CYP2D6₃₉₃₋₄₁₂, CYP2D6₁₉₂₋₂₀₆, CYP2D6₅₋₁₉, CYP2D6₂₉₃₋₃₀₇ (for DRB1*0301); CYP2D6₂₁₉₋₂₃₃, CYP2D6₂₃₇₋₂₅₁, CYP2D6₁₅₋₂₉ (for DRB3*0202); CYP2D6₂₃₅₋₂₄₉, CYP2D6₃₁₇₋₃₃₁, CYP2D6₂₉₃₋₃₀₇ (for DRB4*0101); CYP2D6₄₂₈₋₄₄₂, CYP2D6₂₃₇₋₂₅₁, CYP2D6₁₄₋₂₈ (for DRB5*0101); CYP2D6₁₉₉₋₂₁₃, CYP2D6₄₅₀₋₄₆₄, CYP2D6₃₀₁₋₃₁₅ (for DRB1*0401); CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₅₋₂₉ (for DRB1*0701). In certain embodiments, the ubiquitous autoantigen is selected from the group consisting of: ACTB₂₀₂₋₂₁₆, ACTB₁₇₀₋₁₈₄, ACTB₂₄₅₋₂₅₉, (for DRB1*0301); ACTB₁₈₇₋₂₀₁, ACTB₁₇₂₋₁₈₆, ACTB₁₃₁₋₁₄₅ (for DRB3*0202); ACTB₁₃₁₋₁₄₅, ACTB₁₇₁₋₁₈₅, ACTB₁₂₉₋₁₄₃ (for DRB4*0101); ACTB₁₆₄₋₁₇₈, ACTB₂₅₋₃₉, ACTB₃₂₃₋₃₃₇ (for DRB5*0101). In certain embodiments, the ubiquitous autoantigen is selected from the group consisting of: ACTB₁₄₆₋₁₆₀, ACTB₁₈₋₃₂, and ACTB₁₇₁₋₁₈₅. In certain embodiments, the ubiquitous autoantigen is selected from the group consisting of: SLA₃₃₄₋₃₄₈, SLA₁₉₆₋₂₁₀, SLA₁₁₅₋₁₂₉, SLA₃₇₃₋₃₈₆, SLA₁₈₆₋₁₉₇ (for DRB1*0301); SLA₃₄₂₋₂₅₆, SLA₁₁₀₋₁₂₄, SLA₂₉₉₋₃₁₃ (for DRB3*0202); SLA₄₉₋₆₃, SLA₂₆₀₋₂₇₄, SLA₁₁₉₋₁₃₃ (for DRB4*0101); SLA₈₆₋₁₀₀, SLA₂₆₋₄₀, SLA₃₃₁₋₃₄₅ (for DRB5*0101); SLA₃₁₇₋₃₃₁, SLA₁₇₁₋₁₈₅, SLA₄₁₇₋₄₃₁ (for DRB1*0401); SLA₃₅₉₋₃₇₃, SLA₂₁₅₋₂₂₉, SLA₁₁₁₋₁₂₅ (for DRB1*0701). In certain embodiments, the ubiquitous autoantigen is selected from the group consisting of: FTCD₄₃₉₋₄₅₃, FTCD₃₈₁₋₃₉₅, FTCD₂₉₇₋₃₁₁ (for DRB3*0202); FTCD₅₂₅₋₅₃₉, FTCD₂₁₈₋₂₃₂, FTCD₄₉₅₋₅₀₉ (for DRB1*0301); FTCD₂₆₂₋₂₇₆, FTCD₃₀₀₋₃₁₄, FTCD₂₅₉₋₂₇₃ (for DRB4*0101); FTCD₄₉₀₋₅₀₄, FTCD₃₈₉₋₄₀₃, FTCD₂₉₅₋₃₀₉ (for DRB5*0101). In certain embodiments, the ubiquitous autoantigen is selected from the group consisting of: FTCD₂₇₁₋₂₈₅, FTCD₄₉₈₋₅₁₂, and FTCD₃₀₁₋₃₁₅. In certain embodiments, the ubiquitous autoantigen is selected from the group consisting of: MPO₃₂₂₋₃₃₆, MPO₇₁₄₋₇₂₈, MPO 617-631 (for DRB3*0202); MPO₅₀₄₋₅₁₈, MPO₄₆₂₋₄₇₆, MPO₆₁₇₋₆₃₁ (for DRB1*0301); MPO₄₄₄₋₄₅₈, MPO₆₈₉₋₇₀₃, MPO₂₄₈₋₂₆₂ (for DRB4*0101); MPO₅₁₁₋₅₂₅, MPO₉₇₋₁₁₁, MPO₆₁₆₋₆₃₀ (for DRB5*0101). In certain embodiments, the complex further comprises a second plurality of antigen-major histocompatibility complexes (MHCs) coupled to the nanoparticle core, each antigen-MHC of the second plurality comprising an antigen. In certain embodiments, the antigen of the second plurality of antigen-major histocompatibility complexes (MHCs) is a second ubiquitous autoantigen. In certain embodiments, the second ubiquitous autoantigen comprises a polypeptide derived from a protein that at steady-state exists in or on an intracellular compartment. In certain embodiments, the intracellular compartment is cytosol. In certain embodiments, the intracellular compartment is a mitochondrion. In certain embodiments, the intracellular compartment is a mitochondrion. In certain embodiments, the second ubiquitous autoantigen is selected from the group consisting of: PDC-E2₃₅₃₋₃₆₇, PDC-E2₇₂₋₈₆ and PDC-E2₄₂₂₋₄₃₆ for DRB3*0202; PDC-E2₃₅₃₋₃₆₇, PDC-E2₈₀₋₉₄ and PDC-E2₅₃₅₋₅₄₉ for DRB5*0101; PDC-E2₆₂₉₋₆₄₈, PDC-E2₁₂₂₋₁₃₅ and PDC-E2₂₄₉₋₂₆₃ for DRB4*0101; and PDC-E2₂₄₉₋₂₆₃ for DRB1*0801. In certain embodiments, the ubiquitous autoantigen is selected from the group consisting of: PDC-E2₄₂₂₋₄₃₆ and PDC-E2₈₀₋₉₄. In certain embodiments, the second ubiquitous autoantigen is selected from the group consisting of: CYP2D6₂₈₄₋₂₉₈, CYP2D6₂₈₉₋₃₀₃, CYP2D6₃₁₈₋₃₃₂, CYP2D6₃₁₃₋₃₃₂, CYP2D6₃₉₃₋₄₁₂, CYP2D6₁₉₂₋₂₀₆, CYP2D6₅₋₁₉, CYP2D6₂₉₃₋₃₀₇ (for DRB1*0301); CYP2D6₂₁₉₋₂₃₃, CYP2D6₂₃₇₋₂₅₁, CYP2D6₁₅₋₂₉ (for DRB3*0202); CYP2D6₂₃₅₋₂₄₉, CYP2D6₃₁₇₋₃₃₁, CYP2D6₂₉₃₋₃₀₇ (for DRB4*0101); CYP2D6₄₂₈₋₄₄₂, CYP2D6₂₃₇₋₂₅₁, CYP2D6₁₄₋₂₈ (for DRB5*0101); CYP2D6₁₉₉₋₂₁₃, CYP2D6₄₅₀₋₄₆₄, CYP2D6₃₀₁₋₃₁₅ (for DRB1*0401); CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₅₋₂₉ (for DRB1*0701). In certain embodiments, the second ubiquitous autoantigen is selected from the group consisting of: ACTB₂₀₂₋₂₁₆, ACTB₁₇₀₋₁₈₄, ACTB₂₄₅₋₂₅₉, (for DRB1*0301); ACTB₁₈₇₋₂₀₁, ACTB₁₇₂₋₁₈₆, ACTB₁₃₁₋₁₄₅ (for DRB3*0202); ACTB₁₃₁₋₁₄₅, ACTB₁₇₁₋₁₈₅, ACTB₁₂₉₋₁₄₃ (for DRB4*0101); ACTB₁₆₄₋₁₇₈, ACTB₂₅₋₃₉, ACTB₃₂₃₋₃₃₇ (for DRB5*0101). In certain embodiments, the second ubiquitous autoantigen is selected from the group consisting of: ACTB₁₄₆₋₁₆₀, ACTB₁₈₋₃₂, and ACTB₁₇₁₋₁₈₅. In certain embodiments, the second ubiquitous autoantigen is selected from the group consisting of: SLA₃₃₄₋₃₄₈, SLA₁₉₆₋₂₁₀, SLA₁₁₅₋₁₂₉, SLA₃₇₃₋₃₈₆, SLA₁₈₆₋₁₉₇ (for DRB1*0301); SLA₃₄₂₋₂₅₆, SLA₁₁₀₋₁₂₄, SLA₂₉₉₋₃₁₃ (for DRB3*0202); SLA₄₉₋₆₃, SLA₂₆₀₋₂₇₄, SLA₁₁₉₋₁₃₃ (for DRB4*0101); SLA₈₆₋₁₀₀, SLA₂₆₋₄₀, SLA₃₃₁₋₃₄₅ (for DRB5*0101); SLA₃₁₇₋₃₃₁, SLA₁₇₁₋₁₈₅, SLA₄₁₇₋₄₃₁ (for DRB1*0401); SLA₃₅₉₋₃₇₃, SLA₂₁₅₋₂₂₉, SLA₁₁₁₋₁₂₅ (for DRB1*0701). In certain embodiments, the second ubiquitous autoantigen is selected from the group consisting of: FTCD₄₃₉₋₄₅₃, FTCD₃₈₁₋₃₉₅, FTCD₂₉₇₋₃₁₁ (for DRB3*0202); FTCD₅₂₅₋₅₃₉, FTCD₂₁₈₋₂₃₂, FTCD₄₉₅₋₅₀₉ (for DRB1*0301); FTCD₂₆₂₋₂₇₆, FTCD₃₀₀₋₃₁₄, FTCD₂₅₉₋₂₇₃ (for DRB4*0101); FTCD₄₉₀₋₅₀₄, FTCD₃₈₉₋₄₀₃, FTCD₂₉₅₋₃₀₉ (for DRB5*0101). In certain embodiments, the second ubiquitous autoantigen is selected from the group consisting of: FTCD₂₇₁₋₂₈₅, FTCD₄₉₈₋₅₁₂, and FTCD₃₀₁₋₃₁₅. In certain embodiments, the second ubiquitous autoantigen is selected from the group consisting of: MPO₃₂₂₋₃₃₆, MPO₇₁₄₋₇₂₈, MPO₆₁₇₋₆₃₁ (for DRB3*0202); MPO₅₀₄₋₅₁₈, MPO₄₆₂₋₄₇₆, MPO₆₁₇₋₆₃₁ (for DRB1*0301); MPO₄₄₄₋₄₅₈, MPO₆₈₉₋₇₀₃, MPO₂₄₈₋₂₆₂ (for DRB4*0101); MPO₅₁₁₋₅₂₅, MPO₉₇₋₁₁₁, MPO₆₁₆₋₆₃₀ (for DRB5*0101). In certain embodiments, the composition further comprises a pharmaceutically acceptable stabilizer, excipient, diluent, or combination thereof. In certain embodiments, the autoimmune or inflammatory disease is type I diabetes. In certain embodiments, the ubiquitous autoantigen is not a polypeptide derived from pre-proinsulin, proinsulin, islet-specific glucose-6-phosphatase (IGRP), glutamate decarboxylase (GAD), islet cell autoantigen-2 (ICA2), or insulin.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 illustrates the expansion of Primary Biliary Cirrhosis (PBC) relevant regulatory T cells by nanoparticles coupled to MHC class II associated with peptides derived from pyruvate dehydrogenase complex-E2 component (PDC-E2) (PBC-relevant peptide-major histocompatibility complex-nanoparticles (pMHC-NPs)). FIG. 1A shows percentage of tetramer+CD4+ T-cells in blood of NOD vs. NOD.c3c4 mice as a function of age. FIG. 1B shows percentage of tetramer+CD4+ T-cells in peripheral blood of pMHC-NP-treated NOD.c3c4 mice as compared to untreated NOD or NOD.c3c4 mice or NOD.c3c4 mice treated with control Cys-NPs. FIG. 1C shows percentages of tetramer+CD4+ T-cells in mice from panel B in various organs at the end of pMHC-NP therapy. FIG. 1D shows percentages of tetramer+CD4+ T-cells in NOD.c3c4 mice treated with type 1 diabetes-relevant pMHC-NPs. FIG. 1E shows percentages of tetramer+CD4+ T-cells in various lymphoid organs and liver of NOD.c3c4 mice treated with NPs coated with one of two different PBC-relevant pMHCs. FIG. 1F shows expression of TR1-like cell surface markers by the tetramer+CD4+ T-cells expanded in NOD.c3c4 mice by pMHC-NP therapy. (FIG. 1G shows cytokine secretion profile of sorted tetramer+CD4+ TR1-like cells vs. tetramer-negative CD4+ T-cells ex vivo upon stimulation with peptide-pulsed DCs.

FIG. 2 illustrates the expansion of PBC relevant regulatory T cells by PBC-relevant pMHC-NPs. (FIGS. 2A-B) Upregulation of TR1-like markers on tetramer+CD4+ T-cells expanded in vivo in response to PDC₁₆₆/IA^{g7}- or PDC₈₂/IA^{g7}-NP therapy FIG. 2A is a representative FACS profiles; FIG. 2B shows average mean fluorescence intensity values. FIG. 2C shows average mean fluorescence intensity values for TR1 cell surface markers on tetramer+CD4+ T-cells arising in 38-44 week-old NOD.c3c4 mice in response to PDC₁₆₆/IA^{g7}-NP therapy.

FIG. 3 illustrates clinical, phenotypic, immunological and pathological features of liver disease in NOD.c3c4 mice. FIG. 3A shows changes in serum TB and ALT levels with age. FIG. 3B shows microscopic scoring system (left) and progression of microscopic scores of disease with age (right). FIG. 3C shows representative CBD images and progression of CBD diameter and scores with age. FIG. 3D shows representative liver images (top) and progression of liver scores and weight with age (bottom). FIG. 3E shows that NOD.c3c4 mice spontaneously develop anti-PDC-E2-specific autoantibodies (left) and ANAs (right). FIG. 3F depicts representative images of liver inflammation by CD4+ and CD8+ T-cells.

FIG. 4 illustrates reversal of disease in a mouse model of PBC using PBC-relevant pMHC-NPs. FIG. 4A shows changes in total bile acid (TBA) and alanine aminotransferase (ALT) levels in serum of NOD.c3c4 mice treated with PDC₁₆₆/IA^{g7}-NPs, PDC₈₂/IA^{g7}-NPs or control (Cys-NP). FIG. 4B shows representative histological images of livers from PDC₁₆₆/IA^{g7}-NPs, PDC₈₂/IA^{g7}-NPs, or Cys-NP-treated NOD.c3c4 mice (top) and average histological scores (bottom). FIG. 4C shows representative macroscopic images of the common bile duct (top), and average common bile duct scores and diameters (bottom). FIG. 4D shows representative macroscopic images of livers (top) and average liver scores and weight (bottom). FIG. 4E shows representative whole body images of NOD.c3c4 mice treated with PDC₁₆₆/IA^{g7}-NP or control Cys-NPs. FIG. 4F shows changes in the titer of anti-mitochondrial (PDC-E2) antibodies and anti-nuclear autoantibodies (ANAs) after treatment (top left two panels and right panel, respectively), and representative images of Hep2 cells stained with sera from pMHC-NP vs Cys-NP-treated NOD.c3c4 mice (bottom). FIG. 4G shows percentages of tetramer+ cells in mice treated starting at 24 weeks of age (until week 38-44). FIGS. 4H and 4I show microscopic (FIG. 4H) and macroscopic scores (FIG. 4I) for the mice studied in FIG. 4G.

FIG. 5 illustrates changes in the circulating frequency of tetramer+CD4+ T-cells in response to periodic re-treatment with PBC relevant nanoparticles. FIG. 5A shows two different mice, and FIG. 5B shows average values corresponding to cohorts of mice treated with PDC₁₆₆/IA^{g7}-NPs or left untreated. FIG. 5C shows percentage of tetramer+CD4+ T-cells, and FIG. 5D shows mean fluorescence intensity staining for TR1 markers in tetramer+CD4+ T-cells from the mice studied in FIG. 5A. FIG. 5E shows average macroscopic CBD and liver scores for the mice studied in FIGS. 5A-D.

FIG. 6 illustrates effects of treatment with PBC-relevant pMHC-NPs or the standard of care in PBC (UDCA) on macroscopic and serum ALT levels (FIG. 6A) and microscopic (FIG. 6B) disease scores when treatment is initiated early on in the disease process. FIG. 6C shows percentages of tetramer+CD4+ T-cells in the mice studied in FIGS. 6A-B. FIGS. 6D and E show effects of treatment with PBC-relevant pMHC-NPs or the standard of care in PBC (UDCA) on macroscopic (FIG. 6D) and microscopic (FIG. 6E) disease scores when treatment is initiated at advanced stages of disease. FIG. 6F shows percentages of tetramer+CD4+ T-cells in the mice studied in FIGS. 6D-E.

FIG. 7 illustrates that PBC-relevant pMHC-NPs expand regulatory B-cells. FIG. 7A shows percentages of tetramer+CD4+ T-cells in mice treated with pMHC-NPs and rat-IgG (control) or blocking rat mAbs against mouse IL-10 or TGF-beta. FIGS. 7B and C show macroscopic (FIG. 7B) and microscopic (FIG. 7C) scores of the mice studied in FIG. 7A. FIG. 7D shows percentages of tetramer+CD4+ T cells in blood and lymphoid organs of NOD.c3c4.scid hosts reconstituted with whole splenocytes from untreated NOD.c3c4 donors and then transfused with splenic CD4+ T-cells from PDC₁₆₆₋₁₈₁/IA 87-NP-treated NOD.c3c4 mice. The latter were either left untreated after CD4+ T-cell transfer or were treated with PDC₁₆₆₋₁₈₁/IA^{g7}-NPs. FIG. 7E shows representative FACS staining histograms (top) and average mean fluorescence intensity values for TR1 markers on tetramer+CD4+vs. tetramer-CD4+ T-cells (bottom) of the hosts treated with PDC₁₆₆₋₁₈₁/IA^{g7}-NPs. FIG. 7F shows macroscopic scores and measurements of the mice studied in A. FIG. 7G shows the cytokine profile of LPS challenged CD11b+ cells isolated from the liver draining (PLN) or non-draining (MLN) lymph nodes of pMHC-NP vs Cys-NP-treated NOD.c3c4 mice. FIG. 7H shows the cytokine profile of liver Kupffer cells from pMHC-NP vs Cys-NP-treated NOD.c3c4 mice. FIG. 7I shows absolute numbers of B-cells in liver draining (PCLN) and non-draining (ILN) lymph nodes or liver of pMHC-NP vs Cys-NP-treated NOD.c3c4 mice. FIG. 7J shows correlation between absolute numbers of B-cells and tetramer+CD4+ T-cells in pMHC-NP-treated mice. (IG. 7K shows IL-10 secretion levels of LPS-challenged B-cells isolated from liver draining and non-draining lymph nodes or liver of pMHC-NP vs Cys-NP-treated NOD.c3c4 mice. FIG. 7L shows representative FACS plots showing conversion of B cells into IL-10-producing Breg cells only in the experiments arising in in hosts treated with pMHC-NPs. FIG. 7M shows percentages of conventional B-cell-derived Breg cells in hosts treated with pMHC-NP or Cys-NP in liver and peripheral lymphoid organs harboring (spleen and PCLN) or lacking (MLN) pMHC-NP-induced TR1-like CD4+ T-cells.

FIG. 8 illustrates that PBC-relevant pMHC-NPs spare systemic immunity. FIG. 8A shows average titers of rVV in ovaries of pMHC-NP-treated vs. untreated NOD.c3c4 female mice 4 and 14 days after a systemic rVV infection. FIG. 8B shows titers of influenza virus in lungs of pMHC-NP-treated vs. untreated NOD.c3c4 female mice 3 and 7 days after infection with the PR8 strain with or without prior priming with the HKx31 strain. FIG. 8C shows clinical scores and body weight changes as a function of infection and treatment. FIG. 8D shows colony-forming units of Listeria in the spleen or liver of pMHC-treated vs untreated NOD or NOD.c3c4 mice on days 3, 7, and 14 after a systemic Listeria infection. FIG. 8E shows serum anti-DNP antibody titers upon KLH-DNP immunization (n=3/group). FIGS. 8F-8K show percentages of tetramer+ cells (FIG. 8F), macroscopic PBC scores and liver weight (FIG. 8G), microscopic PBC scores (FIG. 8H), liver images (FIG. 8I), microscopic tumor scores (FIG. 8J) and survival rates (FIG. 8K) of untreated vs. PDC₁₆₆₋₁₈₁/IA^{g7}-NP-treated NOD.c3c4 (n=5/group) or untreated Balb/c (n=7) injected with CT26 cells. FIGS. 8L-8P show percentages of tetramer+ cells (FIG. 8L), macroscopic (FIG. 8M) and microscopic PBC scores (FIG. 8N), and liver weight and metastasis number (FIG. 8O), and liver images (FIG. 8P) of untreated vs. PDC₁₆₆₋₁₈₁/IA^{g7}-NP-treated NOD.c3c4 (n=5 and 4) or untreated B6 (n=6) injected with B16/F10 cells. FIG. 8Q shows the survival rates of the mice in FIGS. 8L-8P. Data correspond to mean±SEM. P values were compared via Mann-Whitney U except for K, Q (log-rank) or C (two-way ANOVA).

FIG. 9 illustrates expansion of T regulatory cells by PBC-relevant pMHC-NPs in humanized mice. FIG. 9A shows representative tetramer stains in NSG hosts engrafted with PBMCs from DRB4*0101+PBC patients and treated with three different PBC-relevant pMHC-NPs. FIG. 9B shows average percentages and absolute numbers of tetramer+CD4+ cells in responsive vs. unresponsive pMHC-NP-treated mice or untreated littermates. FIG. 9C shows mean fluorescence intensity values (top) and two-dimensional FACS plots (bottom) for the TR1 markers CD49b and LAG3 in the mice studied in FIGS. 9A-B.

FIG. 10 illustrates that PBC-relevant pMHC-NPs are able to treat disease in models of liver autoimmunity distinct from PBC, in particular, primary sclerosing cholangitis (PSC) and autoimmune hepatitis (AIH). FIG. 10A shows percentage of tetramer+CD4+ T-cells in NOD.Abcb4−/− mice in response to pMHC-NP therapy. FIG. 10B shows average Primary Sclerosing Cholangitis scores (top) and representative H&E stained or picrosirius-stained liver sections (bottom), and TBA and ALT (right column) corresponding to the mice studied in FIG. 10A. FIG. 10C shows percentage of tetramer+CD4+ T-cells in NOD mice infected with an hFTCD-encoding Adenovirus (developing AIH) and treated with three different pMHC-NP types. FIG. 10D shows autoimmune hepatitis histopathological scores (top) and representative H&D and picrosirius liver stained sections (bottom) for the mice studied in FIG. 10C. FIG. 10E shows serum ALT levels in the mice in FIG. 10C [n=11, 5, 9 and 10 mice (left to right); 2-3 experiments].

FIG. 11 illustrates that PBC and AIH-relevant pMHC-NPs expand T regulatory cells in a mouse model of PSC. FIG. 11A shows representative FACS staining histograms, and FIG. 11B shows average mean fluorescence intensity values for TR1 markers on tetramer+CD4+vs. tetramer-CD4+ T-cells of NOD.Abcb4−/− mice (spontaneously developing PSC) treated with PBC- or AIH-relevant pMHC-NPs.

FIG. 12 illustrates that PBC relevant pMHC-NPs expand T regulatory cells in a mouse model of AIH. FIG. 12A shows representative FACS staining histograms, and FIG. 12B shows average mean fluorescence intensity values for TR1 markers on tetramer+CD4+vs. tetramer-CD4+ T-cells of NOD mice infected with an adenovirus encoding hFTCD (spontaneously developing AIH) treated with PBC- or AIH-relevant pMHC-NPs.

FIG. 13. illustrates the ability of ubiquitous autoantigen based pMHC-NPs to blunt liver autoimmune disease in an organ rather than disease-specific manner. FIG. 13A shows percentage of tetramer+CD4+ T-cells in NOD.c3c4 mice treated with an AIH relevant pMHC-NP type. FIG. 13B shows average macroscopic liver scores in NOD.c3c4 mice treated with PBC-AIH- or TID-relevant pMHC-NP types. FIG. 13C shows percentage of tetramer+CD4+ T-cells in NOD mice treated with PBC- or AIH-relevant pMHC-NP types.

FIG. 14 illustrates therapeutic effects of PBC-relevant pMHCII-NPs in (NODxB6.Ifng-ARE-Del−/−) F1 mice. FIG. 14A shows percentages of tetramer+CD4+ T-cells in mice (males and females pooled) treated with Cys-NPs or PDC₁₆₆₋₁₈₁/IA^{g7}-NPs. FIG. 14B shows serum TBA and ALT levels in the mice studied in A. FIG. 14C shows microscopic scores of the female mice studied in A (left) and representative H&E (top right) and picrosirius-red-stained liver sections (bottom right). Data in FIGS. 14A-C correspond to n=7 and 12 mice/treatment type, respectively, from 2-3 experiments.

FIG. 15 illustrates that disease-relevant pMHCII-NPs spare general immunity despite harboring PDC-E2 autoreactive TR1-like CD4+ T-cells. FIG. 15A shows average percentages of PDC₁₆₆₋₁₈₁/IA^{g7} tetramer+CD4+ T-cells in blood and lymphoid organs of PDC₁₆₆₋₁₈₁/IA^{g7} in NP-treated NOD.c3c4 mice 7 days after priming with HKx31 strain. Data correspond to n=4 and 5, respectively. FIG. 15B shows average percentages of PDC₁₆₆₋₁₈₁/IA^{g7} tetramer+CD4+ T-cells in blood and lymphoid organs of PDC₁₆₆₋₁₈₁/A^{g7} in NP-treated NOD.c3c4 mice 14 days after infection with Listeria. Data correspond to n=3 and 5 mice, respectively. Data correspond the mean±SEM. P values were compared via Mann-Whitney U.

FIG. 16 illustrates that pMHCII-based nanomedicines displaying epitopes from ubiquitous autoantigens blunt relapsing-remitting EAE in NOD mice. FIG. 16A shows percentage of tetramer+CD4+ cells in blood, spleen and cervical LNs of pMHCII-NP-treated NOD mice with EAE. Data correspond to (from top): (i) MOG₃₆₋₅₀/IA^{g7} tetramer staining for 5 Cys-NP- and 7 MOG₃₆₋₅₀/IA^{g7}-NP-treated mice; (ii) PDC₁₆₆₋₁₈₁/IA^{g7} tetramer staining for 11 Cys-NP-plus 14 BDC_{2.5mi}/IA^{g7}-NP- and 22 PDC₁₆₆₋₁₈₁/IA^{g7}-NP-treated mice (pooled from 2 experiments); (iii) CYPD₃₉₈₋₄₁₂/IA^{g7} tetramer staining for 11 Cys-NP- and 8 CYPD₃₉₈₋₄₁₂/IA^{g7}-NP-treated mice (pooled from 2 experiments); and (iv) BDC2.5 mi/IA^{g7} tetramer staining for 14 PDC₁₆₆₋₁₈₁/IA^{g7}-NP- and 14 BDC_{2.5mi}/IA^{g7}_NP-treated mice. FIG. 16B shows and MFI for TR1 markers in splenic tetramer+CD4+ cells. Data in FIG. 16B correspond to 3 PDC₁₆₆₋₁₈₁/IA^{g7}-NP- and 4 BDC2.5 mi/IA^{g7}-NP-treated mice. FIG. 16C shows EAE scores in NOD mice treated with: (i) Cys-NPs (n=9), BDC_{2.5mi}/IA^{g7}-NPs (n=14), or MOG₃₆₋₅₀/IA^{g7}-NPs (n=8) (top); and (ii) Cys-NPs (n=11, from 2 experiments) PDC₁₆₆₋₁₈₁/IA^{g7}-NPs (n=22, from 2 experiments) or CYPD₃₉₈₋₄₁₂/IA^{g7}-NPs (n=8) (bottom). FIG. 16D shows representative luxol fast blue (LFB)/H&E staining pictures of cerebellum from the mice in FIG. 16C showing infiltration of inflammatory cells and areas of demyelination (white arrows). FIG. 16E shows average blinded histopathological rank order LFB scores for the mice in FIG. 16D. Data correspond to n=5, 7, 6, 8 and 7/NP type, from left to right in the bar diagram. Data correspond to the mean±SEM. P values were calculated using two-way ANOVA (FIG. 16C) or Mann-Whitney U test (FIGS. 16A, B, and E).

FIG. 17 illustrates pMHCII-based nanomedicines displaying epitopes from ubiquitous autoantigens blunt chronic progressive EAE in C57BL/6 mice. FIG. 17A shows percentages of tetramer+CD4+ cells in in blood, spleen and cervical LNs of pMHCII-NP-treated C57BL/6J mice with EAE. Data correspond to samples from 6 Cys-NP- and 7 pMHCII-NP-treated mice per panel, stained with cognate pMHC tetramers (top: MOG₃₈₋₄₉/IA^{b}; middle: PDC₉₄-108/IA^{b}; and bottom: CYPD₃₅₃₋₃₆₇/IA^{b}). FIG. 17B shows and MFI of TR1 markers in splenic tetramer+CD4+ cells. Data in FIG. 17B correspond to 7 pMHCII-NP-treated mice in each panel. FIG. 17C shows EAE scores and % initial body weight changes in pMHCII-NP-treated C57BL/6J mice with EAE. Data corresponds to 6 Cys-NP-, 7 MOG₃₈₋₄₉/IA^{b}-NP-, 7 PDC₉₄-108/IA^{b}-NP- and 7 CYPD₃₅₃₋₃₆₇/IA^{b}-NP-treated mice. FIG. 17D shows average blinded histopathological rank order LFB scores for the mice in FIG. 17D. Data correspond to n=6, 6, 6 and 7/pMHC-NP type, from left to right in the bar diagram. Data correspond to the mean±SEM. P values were calculated using two-way ANOVA (FIG. 17C) or Mann-Whitney U test (FIGS. 17A, B, and D).

FIG. 18 illustrates autoantigen shedding renders peripheral autoreactive T-cells responsive to pMHCII-NP therapy. FIG. 18A shows percentage of tetramer+CD4+ cells in the blood of pMHCII-NP-treated vs. untreated NOD mice before (time 0) and 2.5 and 5 weeks after treatment initiation. Data correspond to (from left to right panels): (i) 5 PDC₁₆₆₋₁₈₁/IA^{g7}-NP treatment (2 experiments); (ii) 5 CYPD₃₉₈₋₄₁₂/IA^{g7}-NP treatment; and (iii) 5 untreated mice (2 experiments). Blood samples were stained with BDC2.5 mi/IA^{g7}, PDC₁₆₆₋₁₈₁/IA^{g7} and CYPD₃₉₈₋₄₁₂/IA^{g7} tetramers. FIG. 18B shows percentages of tetramer+CD4+ cells in different lymphoid organs and liver of NOD mice treated with PDC₁₆₆₋₁₈₁/IA^{g7}-NPs or CYPD₃₉₈₋₄₁₂/IA^{g7}-NPs. PCLN, portal and celiac LNs; PLN: pancreatic LNs; MLN, mesenteric LNs; BM, bone marrow. Data corresponds to 9 untreated (2 experiments), 5 PDC₁₆₆₋₁₈₁/IA^{g7}-NP-treated (2 experiments) and 5 CYPD₃₉₈₋₄₁₂/IA^{g7}-NP-treated mice. FIG. 18C shows percentages of tetramer+CD4+ cells in PCLN, PLN and spleen of DT-treated RIP-DTR-transgenic NOD mice following pMHCII-NP therapy. Data correspond to 6 Cys-NP-, 5 BDC2.5 mi/IA^{g7}-NP-, 5 PDC₁₆₆₋₁₈₁/IA^{g7}-NP- and 5 CYPD₃₉₈₋₄₁₂/IA^{g7}-NP-treated mice from 2 independent experiments. Samples from mice treated with BDC2.5 mi/IA^{g7}-NPs and CYPD₃₉₈₋₄₁₂/IA^{g7}-NPs were stained with both pMHCII tetramers (cognate and non-cognate). Samples from mice treated with Cys-NPs, PDC₁₆₆₋₁₈₁/IA^{g7}-NPs and MOG₃₆₋₅₀/IA^{g7}-NPs were stained with PDC₁₆₆₋₁₈₁/IA^{g7} or MOG₃₆₋₅₀/IA 87 tetramers, respectively. FIG. 18 D shows mean fluorescence intensity (MFI) for TR1 markers in splenic tetramer+CD4+ cells. Data are presented as mean±SEM. P values were calculated using ANOVA (FIG. 18A) or Mann-Whitney U test (FIGS. 18B, C and D).

FIG. 19 illustrates that a mouse model of psoriasis can prime ubiquitous autoantigen, and that administration of ubiquitous autoantigen pMHCII-NP therapy can reduce signs of psoriasis. FIG. 19A shows percent tetramer staining of PDC₁₆₆₋₁₈₁/IA^{g7} T cells. FIG. 19B shows improvements of ear thickness in mice administered, PDC₁₆₆₋₁₈₁/IA^{g7}-NPs.

FIG. 20 illustrates a non-limiting embodiment of a pMHC-NP of this disclosure. FIG. 20A shows an alpha chain of an MHC class II dimer fused to the CH2 and CH3 domain of an immunoglobulin molecule comprising an engineered knob. FIG. 20B shows a beta chain of an MHC class II with an N-terminal ubiquitous polypeptide (E2₁₂₂₋₁₃₅), fused to the beta chain of the MHC class II, and fused to the CH2 and CH3 domains of an immunoglobulin molecule comprising an engineered hole.

TABLE 1 illustrates linkers useful for coupling ubiquitous autoantigen-MHCs to nanoparticles.

TABLES 2, 3, and 4 illustrate percentages and absolute numbers of tetramer+CD4+ T-cells in NSG mice engrafted with PBMCs from DRB4*0101+PBC patients, upon treatment with three different human PBC-relevant pMHC-NP types.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

“Antigen” as used herein refers to all, part, fragment, or segment of a molecule that can induce an immune response in a subject or an expansion of an immune cell, preferably a T or B cell. Antigens can be polypeptides, lipids, carbohydrates, or nucleic acids.

As used herein “individual” is synonymous with “subject” or “patient”. The individual can be diagnosed with a disease. The individual can suspected of having a particular disease based on manifesting at least one symptom of said disease, having a family history of said disease, having a genotype relevant to define risk for said disease, or having one or more phenotypic measurements or “lab tests” at or near a level that would place an individual at risk for the disease. The individual can be a mammal, such as a horse, cat, dog, pig, cow, goat, or sheep. The individual can in certain instances be a human person.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, “polypeptide” refers to a plurality of amino acids joined by peptide bonds having more than about eight amino acid residues. The amino acids of the polypeptide can be naturally occurring or unnatural amino acid residues.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The uaMHC of the uaMHC-NP of the current disclosure, described herein, can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

Any of the nucleic acids encoding the uaMHC or the vectors comprising said nucleic acids can be transferred to a suitable cell line for the production of uaMHC. In certain embodiments, the nucleic acid or vector is stably integrated into the genome of the cell line. Suitable cell lines can be as Vero (ATCC CRL 81) or CHO-K1 (ATCC CRL 61) cells, HeLa cells and L cells. Exemplary eukaryotic cells that can be used to express polypeptides include, but are not limited to, COS cells, including COS 7 cells; 293 cells, including 293-6E cells; CHO cells, including CHO-S and DG44 cells; PER.C6™ cells (Crucell); and NSO cells.

Described herein in certain embodiments, is a composition comprising: a plurality of antigen-major histocompatibility complexes (MHCs), each antigen-MHC of the plurality comprising a ubiquitous autoantigen, that is not a tissue specific antigen, associated with the binding groove of an MHC molecule; and a nanoparticle core possessing a diameter of between 1 and 100 nanometers; wherein the antigen-MHCs are coupled to the nanoparticle core.

Described herein in certain embodiments, is a method of treating an autoimmune or inflammatory disease comprising administering to an individual a therapeutically effective amount of a composition comprising: (a) a plurality of antigen-major histocompatibility complexes (MHCs), each antigen-MHC of the plurality comprising a ubiquitous autoantigen, that is not a tissue specific antigen, associated with the binding groove of an MHC molecule; (b) and a nanoparticle core possessing a diameter of between 1 and 100 nanometers; wherein the antigen-MHCs are coupled to the nanoparticle core.

Described herein in certain embodiments, is a method of treating multiple sclerosis comprising administering to an individual a therapeutically effective amount of a composition comprising: (a) a plurality of antigen-major histocompatibility complexes (MHCs), each antigen-MHC of the plurality comprising a ubiquitous autoantigen, which is not a multiple sclerosis specific antigen, associated with the binding groove of an MHC molecule; (b) and a nanoparticle core, the nanoparticle core possessing a diameter of between 1 and 100 nanometers; wherein the antigen-MHCs are coupled to the nanoparticle core.

Described herein in certain embodiments, is a method of treating type I diabetes comprising administering to an individual a therapeutically effective amount of a composition comprising: (a) a plurality of antigen-major histocompatibility complexes (MHCs), each antigen-MHC of the plurality comprising a ubiquitous autoantigen, which is not a type I diabetes specific antigen, associated with the binding groove of an MHC molecule; and (b) a nanoparticle core, the nanoparticle core possessing a diameter of between 1 and 100 nanometers; wherein the plurality of antigen-MHCs is coupled to the nanoparticle core.

### Ubiquitous Autoantigens

Described herein are nanoparticle compositions and methods useful for treating autoimmune diseases and inflammatory disorders. The nanoparticle compositions comprise a plurality of antigens associated with MHCs coupled to a nanoparticle. The nanoparticle compositions and methods utilize broadly expressed ubiquitous autoantigens to elicit the generation of regulatory T and B lymphocytes.

In a certain aspect, the antigens that are associated with the MHC molecules are ubiquitous autoantigens. Ubiquitous autoantigens are differentiated from tissue specific antigens at least in that they are antigens commonly expressed by a plurality of different cell types that are unrelated. In certain embodiments, a ubiquitous autoantigen is one that is commonly expressed by ontogenically distinct tissues. In certain embodiments, a ubiquitous autoantigen that is one that is expressed in at least two cell types derived from a tissue originating from the list consisting of ectoderm, mesoderm, and endoderm. In certain embodiments, a ubiquitous autoantigen is one that is commonly expressed by functionally distinct tissues. In certain embodiments, a ubiquitous autoantigen is one that is expressed in at least two tissues selected from the list consisting of neural tissue, endocrine tissue, connective tissue, hematopoietic cells, liver issue, cardiac tissue, skin tissue, lung tissue, vascular tissue, intestinal tissue, and stomach tissue. In certain embodiments, a ubiquitous autoantigen is one that is expressed in both neural tissue and liver tissue. In certain embodiments, a ubiquitous autoantigen is one that is expressed in both neural tissue and pancreatic tissue. In a certain embodiment, the ubiquitous autoantigen is a polypeptide derived from a protein that participates in a cellular process common to at least 1, 2, 3, 4, 5, 6, 7, 8, 9, 10, or more cell types. Ubiquitous autoantigens may be sequences that are common to two or more closely related proteins that are recognized as being paralogs or homologs in the same family, yet display differential expression across various tissues. In certain embodiments, the two or more closely related proteins can for example, perform the same or similar function in two different unrelated tissues. In a certain embodiment, the ubiquitous autoantigen is a polypeptide derived from a protein that participates in a cellular process, wherein the cellular process is a metabolic process selected from glycolysis, oxidative phosphorylation, glycogenesis, nucleotide biosynthesis, beta oxidation, and omega oxidation. In certain embodiments, the ubiquitous autoantigen is selected from the list consisting of pyruvate dehydrogenase complex-E2 component (PDC-E2), Cytochrome P450 2D6 (CYP2D6), formimidoyltransferase-cyclodeaminase (FTCD), soluble liver antigen (SLA), actin (ACTB), and myeloperoxidase (MPO).

Ubiquitous autoantigens are often encoded by housekeeping genes that are utilized in a variety of cell types. For example, actin is a cytoskeletal protein that contributes to cell structure, motility, cell division, and vesicle motility and is also ubiquitously expressed. As such, many ubiquitous autoantigens are intracellular and reside in a particular intracellular compartment at a steady-state. An antigen exists at steady-state in the cellular location where the antigen can be found at its highest quantities, determined, for example, by microscopy or cell fractionation. For example, despite the fact that actin can be found extracellularly associated with exosomes the vast majority of actin is found in the cytosol of the cell. Likewise, many antigens may transit through different organelles but reside primarily in a single organelle. For example, many endoplasmic reticulum (ER) resident proteins will transiently transit through the cis-Golgi, but are immediately returned to the ER, where they reside at steady-state.

In certain embodiments, the ubiquitous autoantigens for use with the nanoparticle compositions described herein are polypeptides derived from pyruvate dehydrogenase complex-E2 component (PDC-E2). In certain embodiments, the polypeptide derived from PDC-E2 is any one or more of: PDC-E2₃₅₃₋₃₆₇, PDC-E2₇₂₋₈₆ and PDC-E2₄₂₂₋₄₃₆ for DRB3*0202; PDC-E2₃₅₃₋₃₆₇, PDC-E2₈₀₋₉₄ and PDC-E2₅₃₅₋₅₄₉ for DRB5*0101; PDC-E2₆₂₉₋₆₄₈, PDC-E2₁₂₂₋₁₃₅ and PDC-E2₂₄₉₋₂₆₃ for DRB4*0101; and PDC-E2₂₄₉₋₂₆₃ for DRB1*0801. In certain embodiments, the polypeptide derived from PDC-E2 is any one or more of: PDC-E2₄₂₂₋₄₃₆ and PDC-E2₈₀₋₉₄. In certain embodiments, the polypeptide derived from PDC-E2 is any one or more of: SEQ ID NOs: 1 to 12.

In certain embodiments, the ubiquitous autoantigens for use with the nanoparticle compositions described herein are polypeptides derived from Cytochrome P450 2D6 (CYP2D6). In certain embodiments, the polypeptide derived from CYP2D6 is any one or more of: CYP2D6₂₈₄₋₂₉₈, CYP2D6₂₈₉₋₃₀₃, CYP2D6₃₁₈₋₃₃₂, CYP2D6₃₁₃₋₃₃₂, CYP2D6₃₉₃₋₄₁₂, CYP2D6₁₉₂₋₂₀₆, CYP2D6₅₋₁₉, CYP2D6₂₉₃₋₃₀₇ (for DRB1*0301); CYP2D6₂₁₉₋₂₃₃, CYP2D6₂₃₇₋₂₅₁, CYP2D6₁₅₋₂₉ (for DRB3*0202); CYP2D6₂₃₅₋₂₄₉, CYP2D6₃₁₇₋₃₃₁, CYP2D6₂₉₃₋₃₀₇ (for DRB4*0101); CYP2D6₄₂₈₋₄₄₂, CYP2D6₂₃₇₋₂₅₁, CYP2D6₁₄₋₂₈ (for DRB5*0101); CYP2D6₁₉₉₋₂₁₃, CYP2D6₄₅₀₋₄₆₄, CYP2D6₃₀₁₋₃₁₅ (for DRB1*0401); CYP2D6₄₅₂₋₄₆₆, CYP2D6₅₉₋₇₃, CYP2D6₁₃₀₋₁₄₄, CYP2D6₁₉₃₋₂₁₂, CYP2D6₃₀₅₋₃₂₄, CYP2D6₁₅₋₂₉ (for DRB1*0701). In certain embodiments, the polypeptide derived from CYP2D6 is any one or more of: SEQ ID NOs: 13 to 37.

In certain embodiments, the ubiquitous autoantigens for use with the nanoparticle compositions described herein are polypeptides derived from soluble liver antigen (SLA). In certain embodiments, the polypeptide derived from SLA is any one or more of: SLA₃₃₄₋₃₄₈, SLA₁₉₆₋₂₁₀, SLA₁₁₅₋₁₂₉, SLA₃₇₃₋₃₈₆, SLA₁₈₆₋₁₉₇ (for DRB1*0301); SLA₃₄₂₋₂₅₆, SLA₁₁₀₋₁₂₄, SLA₂₉₉₋₃₁₃ (for DRB3*0202); SLA₄₉₋₆₃, SLA₂₆₀₋₂₇₄, SLA₁₁₉₋₁₃₃ (for DRB4*0101); SLA₈₆₋₁₀₀, SLA₂₆₋₄₀, SLA₃₃₁₋₃₄₅ (for DRB5*0101); SLA₃₁₇₋₃₃₁, SLA₁₇₁₋₁₈₅, SLA₄₁₇₋₄₃₁ (for DRB1*0401); SLA₃₅₉₋₃₇₃, SLA₂₁₅₋₂₂₉, SLA₁₁₁₋₁₂₅ (for DRB1*0701). In certain embodiments, the polypeptide derived from SLA is any one or more of: SEQ ID NOs: 53 to 72.

In certain embodiments, the ubiquitous autoantigens for use with the nanoparticle compositions described herein are polypeptides derived from actin (ACTB). In certain embodiments, the polypeptide derived from ACTB is any one or more of: ACTB₂₀₂₋₂₁₆, ACTB₁₇₀₋₁₈₄, ACTB₂₄₅₋₂₅₉, (for DRB1*0301); ACTB₁₈₇₋₂₀₁, ACTB₁₇₂₋₁₈₆, ACTB₁₃₁₋₁₄₅ (for DRB3*0202); ACTB₁₃₁₋₁₄₅, ACTB₁₇₁₋₁₈₅, ACTB₁₂₉₋₁₄₃ (for DRB4*0101); ACTB₁₆₄₋₁₇₈, ACTB₂₅₋₃₉, ACTB₃₂₃₋₃₃₇ (for DRB5*0101). In certain embodiments, the polypeptide derived from ACTB is any one or more of: ACTB₁₄₆₋₁₆₀,ACTB₁₈₋₃₂, and ACTB₁₇₁₋₁₈₅. In certain embodiments, the polypeptide derived from ACTB is any one or more of: SEQ ID NOs: 38 to 52.

In certain embodiments, the ubiquitous autoantigens for use with the nanoparticle compositions described herein are polypeptides derived from formimidoyltransferase-cyclodeaminase (FTCD). In certain embodiments, the polypeptide derived from FTCD is any one or more of: FTCD₄₃₉₋₄₅₃, FTCD₃₈₁₋₃₉₅, FTCD₂₉₇₋₃₁₁ (for DRB3*0202); FTCD₅₂₅₋₅₃₉, FTCD₂₁₈₋₂₃₂, FTCD₄₉₅₋₅₀₉ (for DRB1*0301); FTCD₂₆₂₋₂₇₆, FTCD₃₀₀₋₃₁₄, FTCD₂₅₉₋₂₇₃ (for DRB4*0101); FTCD₄₉₀₋₅₀₄, FTCD₃₈₉₋₄₀₃, FTCD₂₉₅₋₃₀₉ (for DRB5*0101). In certain embodiments, the polypeptide derived from FTCD is any one or more of: FTCD₂₇₁₋₂₈₅, FTCD₄₉₈₋₅₁₂, and FTCD₃₀₁₋₃₁₅. In certain embodiments, the polypeptide derived from FTCD is any one or more of: SEQ ID NOs: 73 to 87.

In certain embodiments, the ubiquitous autoantigens for use with the nanoparticle compositions described herein are polypeptides derived from myeloperoxidase (MPO). In certain embodiments, the polypeptide derived from MPO is any one or more of: MPO₃₂₂₋₃₃₆, MPO₇₁₄₋₇₂₈, MPO₆₁₇₋₆₃₁ (for DRB3*0202); MPO₅₀₄₋₅₁₈, MPO₄₆₂₋₄₇₆, MPO₆₁₇₋₆₃₁ (for DRB1*0301); MPO₄₄₄₋₄₅₈, MPO₆₈₉₋₇₀₃, MPO₂₄₈₋₂₆₂ (for DRB4*0101); MPO₅₁₁₋₅₂₅, MPO₉₇₋₁₁₁, MPO₆₁₆₋₆₃₀ (for DRB5*0101). In certain embodiments, the polypeptide derived from MPO is any one or more of: SEQ ID NOs: 88 to 99.

Additional ubiquitous autoantigens are listed in Table 5 at the end of this disclosure. In certain embodiments, the ubiquitous autoantigens for use with the nanoparticle compositions described herein are derived from a protein or polypeptide listed in Table 5. In certain embodiments, the ubiquitous autoantigens for use with the nanoparticle compositions described herein is derived from a human homolog to protein or polypeptide listed in Table 5. In certain embodiments, a homologue or human homologue is a protein or polypeptide that displays at least about 75%, 80%, 85%, 90%, 95%, or 98% identity to a protein listed in Table 5.

### Tissue Specific Antigens

The nanoparticle compositions and methods described herein utilize ubiquitous autoantigens that are not tissue specific antigens. Many autoimmune or inflammatory diseases are associated with an immune response directed to a tissue specific antigen. This presents a problem for the production of a medicament to treat an autoimmune or inflammatory disease, since each disease requires a specific medicament that targets that antigen. Alternatively, nonspecific immune inhibitors can be used, but these are associated with significant systemic side-effects.

Tissue specific antigens are often expressed by a tissue or cell type affected by the autoimmune disease, for example a main pathological consequence of multiple sclerosis is demyelination of nervous system tissue, as a consequence tissue specific antigens for multiple sclerosis are largely restricted to the nervous system (e.g., myelin basic protein). Tissue specific antigens are those antigens that are associated with a specific cell or cell type. Tissue specific antigens can perform specialized functions or contribute to specialized tissue structures. In certain embodiments, a tissue specific antigen has expression restricted to any one of the following tissues: neural, kidney, cardiac, lung, liver, small intestine, colon, stomach, muscle, connective, and blood-vessel. In certain embodiments, a tissue specific antigen is restricted to expression of any one of the following cell types: beta cells, alpha cells, B lymphocytes, T lymphocytes, Schwann cells, adrenocortical cells.

Many tissue specific antigens may be expressed at a very low level in other cell or tissue types, but the main source of expression is one specific cell or tissue type. For example, a single cell or tissue type that displays cell specific or tissue type specific expression of a certain gene will express at least 10-fold, 50-fold, 100-fold, 500-fold, 1,000 fold or more of the gene at the mRNA or protein level than any other unrelated cell-type. Additionally, some tissue specific antigens will gain ectopic expression of a cell specific antigen under a pathogenic condition or by an exogenous stimulation. It is intended that merely because a different cell-type may gain ectopic expression under pathological or exogenous conditions the tissue specific nature of the antigen is not lost. For example, insulin is a tissue specific antigen produced by beta cells, yet due to genetic instability, some tumors (known as insulinomas) will express insulin, and under these types of circumstances insulin is still considered tissue specific.

Tissue specific antigens that are not ubiquitous autoantigens are primarily antigens associated with a particular tissue specific autoimmune or inflammatory disease.

In certain embodiments, the autoimmune or inflammatory disease is multiple sclerosis. In certain embodiments, the tissue specific antigen that is not a ubiquitous autoantigen is a polypeptide derived from myelin basic protein, myelin associated glycoprotein, myelin oligodendrocyte protein (MOG), proteolipid protein, oligodendrocyte myelin oligoprotein, myelin associated oligodendrocyte basic protein, oligodendrocyte specific protein, heat shock proteins, an oligodendrocyte specific protein, NOGO A, glycoprotein Po, peripheral myelin protein 22, and/or 2′3′-cyclic nucleotide 3′-phosphodiesterase.

In certain embodiments, the autoimmune or inflammatory disease is type I diabetes. In certain embodiments, the tissue specific antigen that is not a ubiquitous autoantigen is a polypeptide derived from pre-proinsulin, proinsulin, islet-specific glucose-6-phosphatase (IGRP), glutamate decarboxylase (GAD), islet cell autoantigen-2 (ICA2), and/or insulin.

In certain embodiments, the autoimmune or inflammatory disease is Pemphigus Foliaceus (PF) or Pemphigus Vulgaris (PV). In certain embodiments, the tissue specific antigen that is not a ubiquitous autoantigen is a polypeptide derived from desmoglein 3 (DG3) and/or desmoglein 1 (DG1).

In certain embodiments, the autoimmune or inflammatory disease is Neuromyelitis optica spectrum disorder (NMO). In certain embodiments, the tissue specific antigen that is not a ubiquitous autoantigen is a polypeptide derived from aquaporin 4 (AQP4).

In certain embodiments, the autoimmune or inflammatory disease is Arthritis. In certain embodiments, the tissue specific antigen that is not a ubiquitous autoantigen is a polypeptide derived from a heat shock protein, immunoglobulin binding protein, heterogeneous nuclear RNPs, annexin V, calpastatin, type II collagen, glucose-6-phosphate isomerase, elongation factor human cartilage gp39, mannose binding lectin, citrullinated vimentin, type II collagen, fibrinogen, alpha enolase, anti-carbamylated protein (anti-CarP), peptidyl arginine deiminase type 4 (PAD4), BRAF, fibrinogen gamma chain, inter-alpha-trypsin inhibitor heavy chain H1, alpha-1-antitrypsin, plasma protease C1 inhibitor, gelsolin, alpha 1-B glycoprotein, ceruloplasmin, inter-alpha-trypsin inhibitor heavy chain H4, complement factor H, alpha 2 macroglobulin, serum amyloid, C-reactive protein, serum albumin, fibrogen beta chain, serotransferin, alpha 2 HS glycoprotein, vimentin, and/or Complement C3

In certain embodiments, the autoimmune or inflammatory disease is allergic asthma. In certain embodiments, the tissue specific antigen that is not a ubiquitous autoantigen is a polypeptide derived from DERP1 and/or DERP2.

In certain embodiments, the autoimmune or inflammatory disease is inflammatory bowel disease. In certain embodiments, the tissue specific antigen that is not a ubiquitous autoantigen is a polypeptide derived from bacteroides integrase, flagelin, flagellin 2 (Fla-2/Fla-X), or uncharacterized E. coli protein (YIDX).

In certain embodiments, the autoimmune or inflammatory disease is systemic lupus erythematosus disease. In certain embodiments, the tissue specific antigen that is not a ubiquitous autoantigen is a polypeptide H4, H2B, H1′, dsDNA, RNP, Smith (Sm), Sjogren's Syndrome-related Antigen A (SS-A)/Ro, Sjogren's Syndrome-related Antigen B (SS-B)/La, and/or histones. In some embodiments, SS-A includes, but is not limited to, RO60 and RO52. In some embodiments, histones include but are not limited to H4, H2B, H1.

In certain embodiments, the autoimmune or inflammatory disease is atherosclerosis. In certain embodiments, the tissue specific antigen that is not a ubiquitous autoantigen is a polypeptide derived from Apolipoprotein B (ApoB) and/or Apolipoprotein E (ApoE).

In certain embodiments, the autoimmune or inflammatory disease is chronic obstructive pulmonary disease (COPD). In certain embodiments, the tissue specific antigen that is not a ubiquitous autoantigen is a polypeptide derived from elastin.

In certain embodiments, the autoimmune or inflammatory disease is psoriasis. In certain embodiments, the tissue specific antigen that is not a ubiquitous autoantigen is a polypeptide derived from human adamis-like protein 5 (ATL5), cathelicidin antimicrobial peptide (CAP18), and/or ADAMTS-like protein 5 (ADMTSL5).

In certain embodiments, the autoimmune or inflammatory disease is uveitis. In certain embodiments, the tissue specific antigen that is not a ubiquitous autoantigen is a polypeptide derived from arrestin, human retinal S-antigen, and/or interphotoreceptor retinoid-binding protein (IRBP).

In certain embodiments, the autoimmune or inflammatory disease is Sjogren's syndrome. In certain embodiments, the tissue specific antigen that is not a ubiquitous autoantigen is a polypeptide derived from (SS-A)/Ro, (SS-B)/La, RO60, RO52, and/or muscarinic receptor 3 (MR3).

In certain embodiments, the autoimmune or inflammatory disease is scleroderma. In certain embodiments, the tissue specific antigen that is not a ubiquitous autoantigen is a polypeptide derived from centromere autoantigen centromere protein C (CENP-C), DNA topoisomerase I (TOP1), and/or RNA polymerase III.

In certain embodiments, the autoimmune or inflammatory disease is anti-phospholipid syndrome. In certain embodiments, the tissue specific antigen that is not a ubiquitous autoantigen is a polypeptide derived from beta-2-glycoprotein 1 (BG2P1 or APOH).

In certain embodiments, the autoimmune or inflammatory disease is stiff man syndrome. In certain embodiments, the tissue specific antigen that is not a ubiquitous autoantigen is a polypeptide derived from GAD65.

### Antigen-Major Histocompatibility Complexes (MHCs)

The nanoparticle complexes of this disclosure comprise a nanoparticle core, with or without layers and/or coatings, coupled to a ubiquitous autoantigen-MHC. The individual MHC polypeptide(s) and the antigenic (e.g., polypeptide) components form a complex through covalent or non-covalent binding (e.g. through hydrogen bonds, ionic bonds, or hydrophobic bonds). The preparation of such complexes may require varying degrees of manipulation and such methods are well known in the literature. In some aspects, antigenic components can be associated non-covalently with the pocket portion of the MHC component by, for instance, mixing the MHC and antigenic components; this relies on the natural binding affinity between an MHC and an antigen. Alternatively, in some aspects, the MHC component may be covalently associated with the antigenic component using standard procedures, such as, but not limited to, the introduction of known coupling agents or photo affinity labelling (see e.g., Hall et al., Biochemistry 24:5702-5711 (1985)). In certain aspects, an antigenic component may be operatively coupled to the MHC component via peptide linkages or other methods discussed in the literature, including but not limited to, attachment via carbohydrate groups on the glycoproteins, including, e.g., the carbohydrate moieties of the alpha- and/or beta-chains. In particular embodiments, the antigenic component may be attached to the N-terminal or C-terminal end of an appropriate MHC molecule. Alternatively, in certain embodiments, the MHC complex may be recombinantly formed by incorporating the sequence of the antigenic component into a sequence encoding an MHC, such that both retain their functional properties.

Multiple ubiquitous autoantigen-MHCs may be coupled to the same nanoparticle core; these complexes, MHCs, and/or antigens may be the same or different from one another.

### Major Histocompatibility Molecules

The ubiquitous autoantigens described herein are associated with MHC molecules (to form the ubiquitous autoantigen-MHC), and coupled to nanoparticles. The antigens are bound to the binding grove of the MHC molecule. MHC molecules primarily bind antigens that are polypeptides, but polypeptides can comprise modifications such as lipidation, glycosylation, phosphorylation and the like. The MHC molecule can be an MHC class I molecule (MHCI) or an MHC class II molecule (MHCII). MHC class I molecules bind polypeptides between 8-10 amino acid residues in their binding groove, as the binding groove is closed on either side. MHC class II molecules, including those described herein, bind polypeptides at least 8 amino acids residues in length, but can bind longer peptides, with lengths of 9, 10, 11, 12, 13, 14, 15, 16, 17, 18, 19 amino acid residues or longer, as the binding groove is open on either side.

For use with human individuals the MHC molecules utilized herein are human (also referred to as human leukocyte antigens, abbreviated “HLA”). In certain embodiments, the MHC class I molecule is a classical or a non-classical MHC class I molecule HLA-A, HLA-B, HLA-C, HLA-E, CD1d, or a fragment or biological equivalent thereof. In certain embodiments, the MHC class II molecule is an HLA-DRB1, HLA-DRB3, HLA-DRB4, HLA-DRB5, HLA-DQB1, or HLA-DPB1, or a fragment or biological equivalent thereof. In some embodiments, the antigen-MHC (pMHC) can be a single chain construct. In some embodiments, the pMHC can be a dual-chain construct. In the case of a class II MHC, the beta chain of HLA will generally be non-covalently bound with an appropriate alpha chain to form a dual chain heterodimer with the alpha chain paired to the beta chain. Generally, the alpha chain of the MHC class II exhibits a much lower degree of polymorphism, for example, the DR alpha chain.

Since MHC class II complexes are heterodimers comprising an alpha and a beta chain, the heterodimers can have problems forming under some conditions, or are inherently unstable in some circumstances. When MHC class II molecules are deployed in a method or composition herein, the MHC molecules can further comprise a knob-in-hole architecture. In general, the alpha or beta chain is fused to an antibody C_{H}2 and C_{H}3 domain that has been modified to comprise a protuberance, while the corresponding other alpha or beta chain of the heterodimer is fused to an antibody C_{H}2 and C_{H}3 domain that has been modified to comprise a cavity.

As used herein, “knob-in-hole” or “knob-into-hole” refers to a polypeptidyl architecture requiring a protuberance (or “knob”) at an interface of a first polypeptide and a corresponding cavity (or a “hole”) at an interface of a second polypeptide, such that the protuberance can be positioned in the cavity so as to promote heterodimer formation. Protuberances may be constructed by replacing small amino acid side chains from the interface of the first polypeptide with larger side chains (e.g., phenylalanine or tyrosine). Cavities of identical or similar size to the protuberances may be created in the interface of the second polypeptide by replacing large amino acid side chains with smaller ones (e.g., alanine or threonine). The protuberances and cavities can be made by synthetic means such as by altering the nucleic acid encoding the polypeptides or by peptide synthesis, using routine methods by one skilled in the art. In some embodiments, the interface of the first polypeptide is located on an Fc domain in the first polypeptide; and the interface of the second polypeptide is located on an Fc domain on the second polypeptide. Knob-in-hole heterodimers and methods of their preparation and use are disclosed in U.S. Pat. Nos. 5,731,168; 5,807,706; 5,821,333; 7,642,228; 7,695,936; 8,216,805; and 8,679,785; and in Merchant et al., Nature Biotechnology, 1998, 16:677-681, all of which are incorporated by reference herein in their entirety.

Alternatively or in addition any of the antigens described herein can comprise a cysteine residue that interacts with a cysteine residue (engineered or natural) of an MHC class II alpha or beta chain. This is commonly known as a cysteine trap.

A cysteine trap can be utilized to stabilize a heterodimer described herein. Cysteine trapping involves forming covalently joined polypeptide complexes from unbound polypeptide partners. In some embodiments, cysteine trapping comprises introducing a cysteine at a strategically selected position within the interaction interface of the polypeptide partners to form a stabilized polypeptide complex. In some embodiments, cysteine trapping may stabilize the polypeptide complex to favor a specific conformation and to prevent dissociation. Cysteine trapping is also referred to as disulfide trapping and disulfide crosslinking. Examples of methods and applications of cysteine trapping are reviewed in Kufareva, et al., Methods Enzymol. 570:389-420 (2016). In the context of MHC, a cysteine is engineered into a polypeptide that is known or suspected to associate in the binding groove of an MHC class II dimer. A cysteine is then engineered in or near the binding groove such that, when the polypeptide associates with the binding groove, the binding groove cysteine can come into proximity and form a disulfide linkage with a polypeptide cysteine.

Provided herein, in one aspect, are isolated heterodimers comprising at least one first polypeptide and at least one second polypeptide, wherein the first polypeptide and the second polypeptide meet at an interface, wherein the interface of the first polypeptide comprises an engineered protuberance which is positionable in an engineered cavity in the interface of the second polypeptide; and (i) the first polypeptide comprises an MHC class II al domain, an MHC class II α2 domain, or a combination thereof; and the second polypeptide comprises an MHC class II β1 domain, an MHC class II β2 domain, or a combination thereof; or (ii) the first polypeptide comprises an MHC class II β1 domain, an MHC class II β2 domain, or a combination thereof; and the second polypeptide comprises an MHC class II al domain, an MHC class II α2 domain, or a combination thereof. The first polypeptide, the second polypeptide, or both can comprise an antibody C_{H}3 domain fused to the polypeptide. Optionally, the first polypeptide, the second polypeptide, or both comprise an antibody C_{H}2 domain located between the MHC (α or β chain) and the C_{H}3 domain. In certain embodiments, the first polypeptide comprises an antibody C_{H}3 domain, and the antibody C_{H}3 domain comprises at least one mutation selected from the list consisting of S354C, T366W, and both S354C and T366W (EU numbering). In certain embodiments, the second polypeptide comprises an antibody CH3 domain, and the antibody CH3 domain comprises at least one mutation selected from the list consisting of Y349C, T366S, L368A, Y407V (EU numbering), and combinations thereof. In further embodiments, the isolated heterodimer comprises a ubiquitous autoantigen, optionally covalently bound to either the first or the second polypeptide. Optionally, the ubiquitous autoantigen comprises a cysteine residue that interacts with a cysteine residue in either the first or second polypeptide to create a cysteine trap.

In one aspect, one polypeptide of the heterodimer comprises an MHC class II al domain, an MHC class II α2 domain, or a combination thereof; and at least one engineered protuberance. In some embodiments, the at least one engineered protuberance is not located at the MHC class II α1 domain or the MHC class II α2 domain. In some embodiments, the engineered protuberance is located at an antibody C_{H}3 domain fused to the polypeptide. In some embodiments, the polypeptide optionally comprises an antibody C_{H}2 domain located between an MHC class II α2 domain and the C_{H}3 domain with an engineered protuberance. In certain embodiments, the polypeptide comprises an antibody C_{H}3 domain, and the antibody C_{H}3 domain comprises at least one mutation selected from the list consisting of S354C, T366W, and both S354C and T366W (EU numbering). In further embodiments, the polypeptide comprises a ubiquitous autoantigen. Optionally, the ubiquitous autoantigen comprises a cysteine residue that interacts with a cysteine residue in either an MHC α1 or β1 domain to create a cysteine trap.

In one aspect, one polypeptide of the heterodimer comprises an MHC class II β1 domain, an MHC class II β2 domain, or a combination thereof; and at least one engineered protuberance. In some embodiments, the at least one engineered protuberance is not located at the MHC class II β1 domain or the MHC class II β2 domain. In some embodiments, the engineered protuberance is located at an antibody CH3 domain fused to the polypeptide. In some embodiments, the polypeptide optionally comprises an antibody C_{H}2 domain located between an MHC class II β2 domain and the C_{H}3 domain with an engineered protuberance. In certain embodiments, the polypeptide comprises an antibody C_{H}3 domain, and the antibody CH3 domain comprises at least one mutation selected from the list consisting of S354C, T366W, and both S354C and T366W (EU numbering). In further embodiments, the polypeptide comprises a ubiquitous autoantigen. Optionally, the ubiquitous autoantigen comprises a cysteine residue that interacts with a cysteine residue in either an MHC α1 or β1 domain to create a cysteine trap.

In one aspect, one polypeptide of the heterodimer comprises an MHC class II al domain, an MHC class II α2 domain, or a combination thereof; and at least one engineered cavity. In some embodiments, the at least one engineered cavity is not located at the MHC class II al domain or the MHC class II α2 domain. In some embodiments, the engineered cavity is located at an antibody C_{H}3 domain fused to the polypeptide. In some embodiments, the polypeptide optionally comprises an antibody C_{H}2 domain located between an MHC class II α2 domain and the C_{H}3 domain with an engineered cavity. In certain embodiments, the polypeptide comprises an antibody C_{H}3 domain, and the antibody C_{H}3 domain comprises at least one mutation selected from the list consisting of Y349C, T366S, L368A, Y407V (EU numbering), and combinations thereof. In further embodiments, the polypeptide comprises a ubiquitous autoantigen. Optionally, the ubiquitous autoantigen comprises a cysteine residue that interacts with a cysteine residue in either an MHC al or β1 domain to create a cysteine trap.

In one aspect, one polypeptide of the heterodimer comprises an MHC class II β1 domain, an MHC class II β2 domain, or a combination thereof; and at least one engineered cavity. In some embodiments, the at least one engineered cavity is not located at the MHC class II β1 domain or the MHC class II β2 domain. In some embodiments, the engineered cavity is located at an antibody C_{H}3 domain fused to the polypeptide. In some embodiments, the polypeptide optionally comprises an antibody C_{H}2 domain located between an MHC class II β2 domain and the C_{H}3 domain with an engineered cavity. In certain embodiments, the polypeptide comprises an antibody C_{H}3 domain, and the antibody C_{H}3 domain comprises at least one mutation selected from the list consisting of Y349C, T366S, L368A, Y407V (EU numbering), and combinations thereof. In further embodiments, the polypeptide comprises a ubiquitous autoantigen. Optionally, the ubiquitous autoantigen comprises a cysteine residue that interacts with a cysteine residue in either an MHC al or β1 domain to create a cysteine trap.

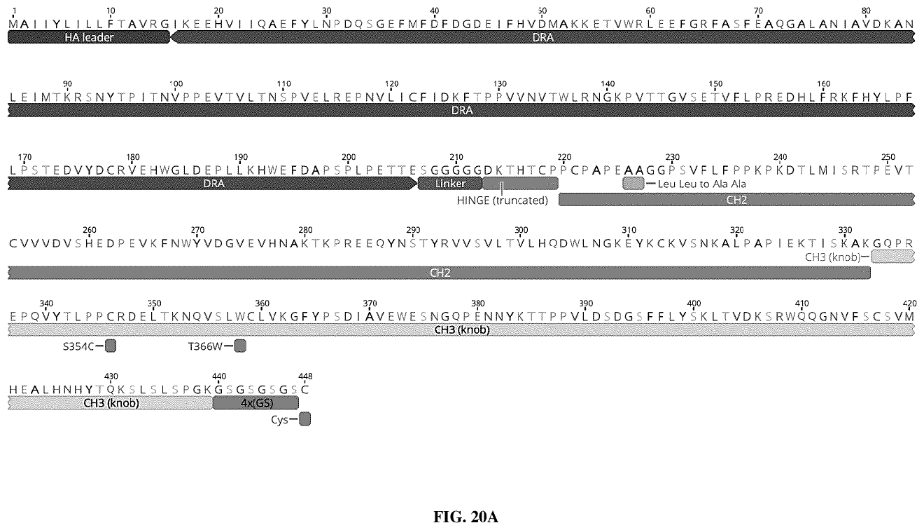

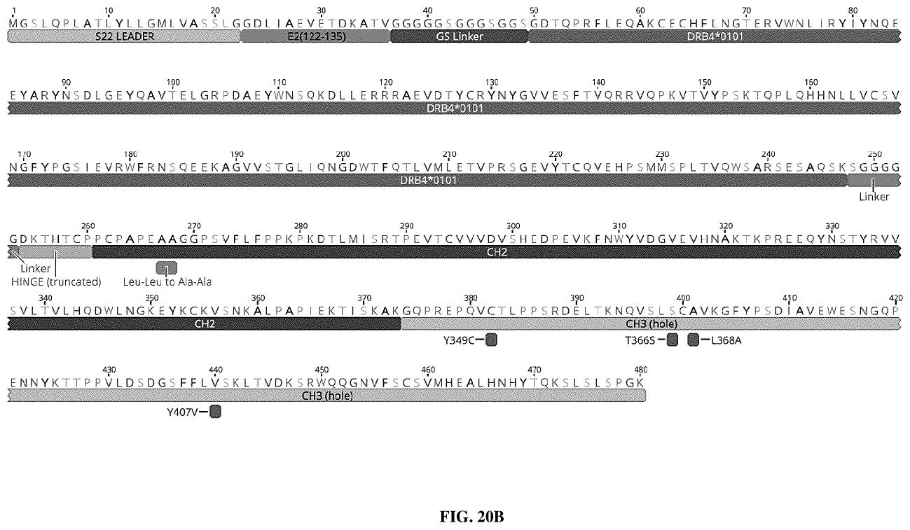

FIGS. 20A and B show non-limiting embodiments of an engineered uaMHC comprising an engineered cavity and an engineered protuberance. FIG. 20A shows a human MHC class II alpha chain fused to an immunoglobulin CH2 and CH3 domain, SEQ ID NO: 103. The CH3 domain comprises an engineered knob that has been created by substituting two amino acids S354C and T366W. The alpha chain comprises an optional c-terminal cysteine to allow for conjugation to a functionalized linker, however this c-terminal cysteine can be alternatively included on the beta chain. FIG. 20B shows a human MHC class II beta chain fused to an immunoglobulin CH2 and CH3 domain, SEQ ID NO: 104. The CH3 domain comprises an engineered hole that has been created by substituting four amino acids Y349C, T366S, L368A and Y407V. The beta chain also comprises a ubiquitous autoantigen (PDC-E2₁₂₂₋₁₃₅) that is covalently coupled to the beta chain by a peptide linker. The protuberance-cavity interaction favors assembly and allows for purification of intact uaMHC heterodimers using standard techniques to purify immunoglobulin. Once purified the uaMHC can be coupled to a suitable nanoparticle through a functionalized linker molecule (E.g., functionalized PEG molecules). In certain embodiments, the alpha chain of MHC heterodimer comprises an amino acid sequence at least about 90%, 95%, 97%, 98%, 99%, or 100% identical to SEQ ID NO: 103. In certain embodiments, the beta chain of MHC heterodimer comprises an amino acid sequence at least about 90%, 95%, 97%, 98%, 99%, or 100% identical to SEQ ID NO: 104. In certain embodiments, the MHC heterodimer comprises an alpha chain at least about 90%, 95%, 97%, 98%, 99%, or 100% identical to SEQ ID NO: 103; and a beta chain at least about 90%, 95%, 97%, 98%, 99%, or 100% identical to SEQ ID NO: 104. In certain embodiments the alpha chain and the beta chain are at least about 90%, 95%, 97%, 98%, 99%, or 100% identical to SEQ ID NO: 103 or 104, and any one or more of the CH2 and CH3 domain of SEQ ID NO: 103, the CH2 and CH3 domain of SEQ ID NO: 103, and/or the ubiquitous autoantigen is identical to that disclosed in SEQ ID NO: 104. In certain embodiments the alpha chain and the beta chain are at least about 90%, 95%, 97%, 98%, 99%, or 100% identical to SEQ ID NO: 103 or 104, and the ubiquitous autoantigen is identical to that disclosed in SEQ ID NO: 104. Those of skill in the art will recognize that the alpha and beta chains of human MHC are highly polymorphic, and thus can tolerate a relatively high degree of variability. One of skill in the art will also be able to substitute the alpha and beta chains of the MHC shown in SEQ ID NOs: 102 and 104 for a different HLA allele, and an appropriate ubiquitous autoantigen that is able to bind the specific substituted allele. Such HLA allele and ubiquitous autoantigen pairings are disclosed elsewhere in the application, for example, at the sequence listing at the end of this application.

### Nanoparticles

The ubiquitous autoantigen-MHCs are coupled to a nanoparticle core (uaMHC-NP). The nanoparticle can be made from a variety of materials. In certain embodiments, the nanoparticle is non-liposomal and/or has a solid core. In certain embodiments, the solid core can be a metal or a metal oxide. In certain embodiments, the solid core can be iron, iron oxide, or gold. The solid core can be a high density core such that the density is greater than about 2.0 g/cm³, about 3.0 g/cm³, about 4.0 g/cm³, about 5.0 g/cm³, about 6.0 g/cm³, or about 7.0 g/cm³. In certain embodiments, the density of the solid core is between about 4.0 g/cm³ and about 8.0 g/cm³. In certain embodiments, the density of the solid core is between about 5.0 g/cm³ and about 8.0 g/cm³. In certain embodiments, the density of the solid core is between about 5.0 g/cm³ and about 7.0 g/cm³. In certain embodiments, the density of the solid core is between about 5.0 g/cm³ and about 6.0 g/cm³.

The nanoparticle core of the uaMHC-NP comprises, or consists essentially of, or yet further consists of a core, for example a solid core, a metal core, a dendrimer core, a polymeric micelle nanoparticle core, a nanorod, a fullerene, a nanoshell, a coreshell, a protein-based nanostructure or a lipid-based nanostructure. In some aspects, the nanoparticle core is bioabsorbable and/or biodegradable. In some aspects, the nanoparticle core is a dendrimer nanoparticle core comprising, or alternatively consisting essentially thereof, or yet further consisting of a highly branched macromolecule having a tree-like structure growing from a core. In further aspects, the dendrimer nanoparticle core may comprise, or alternatively consist essentially thereof, or yet further consist of a poly(amidoamine)-based dendrimer or a poly-L-lysine-based dendrimer. In certain aspects, the nanoparticle core is a polymeric micelle core comprising, or alternatively consisting essentially thereof, or yet further consisting of an amphiphilic block co-polymer assembled into a nano-scaled core-shell structure. In further aspects, the polymeric micelle core comprises, or alternatively consists essentially thereof, or yet further consists of a polymeric micelle produced using polyethylene glycol-diastearoylphosphatidylethanolamine block copolymer. In a further aspect, the nanoparticle core comprises, or alternatively consists essentially of, or yet further consists of a metal. In another aspect, the nanoparticle core is not a liposome. Additional examples of core materials include but are not limited to, standard and specialty glasses, silica, polystyrene, polyester, polycarbonate, acrylic polymers, polyacrylamide, polyacrylonitrile, polyamide, fluoropolymers, silicone, celluloses, silicon, metals (e.g., iron, gold, silver), minerals (e.g., ruby), nanoparticles (e.g., gold nanoparticles, colloidal particles, metal oxides, metal sulfides, metal selenides, and magnetic materials such as iron oxide), and composites thereof. In some embodiments, an iron oxide nanoparticle core comprises iron (II, III) oxide. The core could be of homogeneous composition, or a composite of two or more classes of material depending on the properties desired. In certain aspects, metal nanoparticles will be used. These metal particles or nanoparticles can be formed from Au, Pt, Pd, Cu, Ag, Co, Fe, Ni, Mn, Sm, Nd, Pr, Gd, Ti, Zr, Si, and In, precursors, their binary alloys, their ternary alloys and their intermetallic compounds. See U.S. Pat. No. 6,712,997, which is incorporated herein by reference for such disclosure. In certain embodiments, the compositions of the core and layers (described below) may vary provided that the nanoparticles are biocompatible and bioabsorbable. The core could be of homogeneous composition, or a composite of two or more classes of material depending on the properties desired. In certain aspects, metal nanospheres will be used. These metal nanoparticles can be formed from Fe, Ca, Ga and the like. In certain embodiments, the nanoparticle comprises, or alternatively consists essentially of, or yet further consists of a core comprising metal or metal oxide such as gold or iron oxide.

In another aspect, provided herein are uaMHC-NPs comprising at least one ubiquitous autoantigen-MHC described herein and a nanoparticle, wherein the nanoparticle is non-liposomal and has an iron oxide core.

In another aspect, provided herein are uaMHC-NPs comprising at least one ubiquitous autoantigen-MHC described herein and a nanoparticle, wherein the nanoparticle is non-liposomal and has a gold core.

In another aspect, provided herein are uaMHC-NPs comprising at least one ubiquitous autoantigen-MHC herein and a nanoparticle, wherein the nanoparticle is non-liposomal and has an iron oxide core; and the at least one ubiquitous autoantigen-MHC is covalently linked to the nanoparticle through a linker.

In some aspects, the nanoparticle core has a diameter selected from the group of from about 1 nm to about 100 nm; from about 1 nm to about 75 nm; from about 1 nm to about 50 nm; from about 1 nm to about 25 nm; from about 5 nm to about 100 nm; from about 5 nm to about 50 nm; from about 5 nm to about 40 nm; from about 5 nm to about 30 nm; from about 5 nm to about 25 nm; or from about 5 nm to about 20 nm. In some embodiments, the nanoparticle core has a dimeter from about 10 nm to about 100 nm; from about 10 nm to about 50 nm; from about 10 nm to about 40 nm; from about 10 nm to about 30 nm; from about 10 nm to about 25 nm; or from about 10 nm to about 20 nm. In certain embodiments, the nanoparticle core has a diameter greater than about 1 nm, 2 nm, 5 nm, 10 nm, 15 nm, 20 nm, 25 nm, 30 nm, 40 nm, or 50 nm. In certain embodiments, the nanoparticle core has a diameter less than about 100 nm, 75 nm, 50 nm, 40 nm, 30 nm, 20 nm, or 15 nm.

In some aspects, the nanoparticle core is a dendrimer nanoparticle core comprising, or alternatively consisting essentially thereof, or yet further consisting of a highly branched macromolecule having a tree-like structure growing from a core. In further aspects, the dendrimer nanoparticle may comprise, or alternatively consist essentially thereof, or yet further consist of a poly(amidoamine)-based dendrimer or a poly-L-lysine-based dendrimer. In certain aspects, the nanoparticle core is a polymeric micelle core comprising, or alternatively consisting essentially thereof, or yet further consisting of an amphiphilic block co-polymer assembled into a nano-scaled core-shell structure. In further aspects, the polymeric micelle core may comprise, or alternatively consist essentially thereof or yet further consist of, a polymeric micelle produced using polyethylene glycol-diastearoylphosphatidylethanolamine block copolymer. The dendrimer core or polymeric micelle core may further comprise an outer coating or layer as described herein.

In certain embodiments, specific means of synthesis of dendrimer nanoparticles or nanoparticles with a dendrimer nanoparticle core may require that metal ions are extracted into the interior of dendrimers and then subsequently chemically reduced to yield nearly size-monodispersed particles having dimensions of less than 3 nm, such as the method disclosed in Crooks et al., “Synthesis, Characterization, and Applications of Dendrimer-Encapsulated Nanoparticles”. The Journal of Physical Chemistry B (109): 692-704 (2005), wherein the resulting dendrimer core component serves not only as a template for preparing the nanoparticle but also to stabilize the nanoparticle, making it possible to tune solubility, and provides a means for immobilization of the nanoparticle on solid supports.

The nanoparticle cores typically consist of a substantially spherical core and optionally one or more layers or coatings. The core may vary in size and composition as described herein. In addition to the core, the particle may have one or more layers to provide functionalities appropriate for the applications of interest. The thicknesses of layers, if present, may vary depending on the needs of the specific applications. For example, layers may impart useful optical properties.

Layers may also impart chemical or biological functionalities, referred to herein as chemically active or biologically active layers. These layers typically are applied on the outer surface of the particle and can impart functionalities to the pMHC-NPs. The layer or layers may typically range in thickness from about 0.001 micrometers (1 nanometer) to about 10 micrometers or more (depending on the desired particle diameter) or from about 1 nm to 5 nm, from about 1 nm to about 10 nm, from about 1 nm to about 40 nm, from about 15 nm to about 25 nm, or about 20 nm, and ranges in between.

The layer or coating may comprise, or alternatively consist essentially of, or yet further consist of a biodegradable sugar or other polymer. Examples of biodegradable layers include but are not limited to dextran; poly(ethylene glycol); poly(ethylene oxide); mannitol; poly(esters) based on polylactide (PLA), polyglycolide (PGA), polycaprolactone (PCL); poly(hydroxalkanoate) of the PHB-PHV class; and other modified poly(saccharides) such as starch, cellulose and chitosan. Additionally, the nanoparticle may include a layer with suitable surfaces for attaching chemical functionalities for chemical binding or coupling sites.

### Surface Valency and Density of Antigen-MHCs

The ubiquitous autoantigen-MHCs described herein are coupled to the nanoparticle at a certain valency. Valency is the number of pMHCs per nanoparticle core. In certain embodiments, the valency of the nanoparticle may range between about 1 pMHC per nanoparticle core to about 6,000 pMHCs per nanoparticle core. In certain embodiments, the valency of the nanoparticle may range between about 10 pMHCs per nanoparticle core to about 6,000 pMHCs per nanoparticle core. In certain embodiments, the valency of the nanoparticle may range between about 50 pMHCs per nanoparticle core to about 6,000 pMHCs per nanoparticle core. In certain embodiments, the valency of the nanoparticle may range between about 1 pMHC per nanoparticle core to about 5000, about 4000, about 3000, about 2000, or about 1000 pMHCs per nanoparticle core. In certain embodiments, the valency of the nanoparticle may range between about 10 pMHCs per nanoparticle core to about 5000, about 4000, about 3000, about 2000, or about 1000 pMHCs per nanoparticle core. In certain embodiments, the valency of the nanoparticle may range between about 50 pMHC per nanoparticle core to about 5000, about 4000, about 3000, about 2000, or about 1000 pMHCs per nanoparticle core. In certain embodiments, the valency of the nanoparticle may range between about 1 pMHC to per nanoparticle core to about 1000 pMHCs per nanoparticle core, or between about 10:1 to about 1000:1, or between about 11:1 to about 1000:1, or between about 12:1 to about 1000:1. In certain embodiments, the valency (antigen-MHC to nanoparticle core) may range between about 10:1 to about 500:1, or between about 11:1 to about 500:1, or between about 12:1 to about 500:1. In certain embodiments, the valency (antigen-MHC to nanoparticle core) may range between about 10:1 to about 200:1, or between about 11:1 to about 200:1, or between about 12:1 to about 200:1. In certain embodiments, the valency (antigen-MHC to nanoparticle core) may range between about 10:1 to about 150:1, or between about 11:1 to about 150:1, or between about 12:1 to about 150:1. In certain embodiments, the valency (antigen-MHC to nanoparticle core) may range between about 10:1 to about 100:1, or between about 11:1 to about 100:1, or between about 12:1 to about 100:1. In certain embodiments, the valency (antigen-MHC to nanoparticle core) may range between about 10:1 to about 200:1, between about 20:1 to about 200:1, between about 30:1 to about 200:1, between about 40:1 to about 200:1, or between about 50:1 to about 200:1. In certain embodiments, the valency (antigen-MHC to nanoparticle core) may range between about 10:1 to about 150:1, between about 20:1 to about 150:1, between about 30:1 to about 200:1, between about 40:1 to about 150:1, or between about 50:1 to about 150:1. In certain embodiments, the valency (antigen-MHC to nanoparticle core) may range between about 10:1 to about 100:1, between about 20:1 to about 100:1, between about 30:1 to about 100:1, between about 40:1 to about 100:1, or between about 50:1 to about 100:1.

In some aspects, the nanoparticle core has a defined valency per surface area of the core, also referred to herein as “density.” In these aspects, the pMHC density per nanoparticle is from about 0.025 pMHC/100 nm² to about 100 pMHC/100 nm² of the surface area of the nanoparticle core, or alternatively from about 0.406 pMHC/100 nm² to about 50 pMHC/100 nm²; or alternatively from about 0.05 pMHC/100 nm² to about 25 pMHC/100 nm². In certain aspects, the pMHC density per nanoparticle is from about 0.4 pMHC/100 nm² to about 25 pMHC/100 nm², or from about 0.4 pMHC/100 nm² to about 20 pMHC/100 nm², or from about 0.4 pMHC/100 nm² to about 15 pMHC/100 nm², or from about 0.4 pMHC/100 nm² to about 14 pMHC/100 nm², or from about 0.4 pMHC/100 nm² to about 13 pMHC/100 nm², or from about 0.4 pMHC/100 nm² to about 12 pMHC/100 nm², or from about 0.4 pMHC/100 nm² to about 11.6 pMHC/100 nm², or from about 0.4 pMHC/100 nm² to about 11.5 pMHC/100 nm², or from about 0.4 pMHC/100 nm² to about 11 pMHC/100 nm², or from about 0.4 pMHC/100 nm² to about 10 pMHC/100 nm², or from about 0.4 pMHC/100 nm² to about 9 pMHC/100 nm², or from about 0.4 pMHC/100 nm² to about 8 pMHC/100 nm², or from about 0.4 pMHC/100 nm² to about 7 pMHC/100 nm², or from about 0.4 pMHC/100 nm² to about 6 pMHC/100 nm², or from about 0.4 pMHC/100 nm² to about 5 pMHC/100 nm², or from about 0.4 pMHC/100 nm² to about 4 pMHC/100 nm², or from about 0.4 pMHC/100 nm² to about 3 pMHC/100 nm², or from about 0.4 pMHC/100 nm² to about 2.5 pMHC/100 nm², or from about 0.4 pMHC/100 nm² to about 2 pMHC/100 nm², or from about 0.4 pMHC/100 nm² to about 1.5 pMHC/100 nm².

In another aspect, the nanoparticle may have a pMHC density of from about 0.22 pMHC/100 nm² to about 10 pMHC/100 nm², or from about 0.22 pMHC/100 nm² to about 9 pMHC/100 nm², or from about 0.22 pMHC/100 nm² to about 8 pMHC/100 nm², or from about 0.22 pMHC/100 nm² to about 7 pMHC/100 nm², or from about 0.22 pMHC/100 nm² to about 6 pMHC/100 nm², or from about 0.22 pMHC/100 nm² to about 5 pMHC/100 nm², or from about 0.22 pMHC/100 nm² to about 4 pMHC/100 nm², or from about 0.22 pMHC/100 nm² to about 3 pMHC/100 nm², or from about 0.22 pMHC/100 nm² to about 2 pMHC/100 nm², or from about 0.22 pMHC/100 nm² to about 1.5 pMHC/100 nm². In some aspects, the nanoparticle has a pMHC density of from about 0.22 pMHC/100 nm² to about 10 pMHC/100 nm², or 0.24 pMHC/100 nm² to about 9 pMHC/100 nm², or from about 0.26 pMHC/100 nm² to about 8 pMHC/100 nm², or from about 0.28 pMHC/100 nm² to about 7 pMHC/100 nm², or from about 0.24 pMHC/100 nm² to about 4 pMHC/100 nm², or from about 0.5 pMHC/100 nm² to about 3 pMHC/100 nm², or from about 0.6 pMHC/100 nm² to about 1.5 pMHC/100 nm². In a further aspect, the nanoparticle has a pMHC density of from about 0.4 pMHC/100 nm² to about 1.3 pMHC/100 nm², or alternatively from about 0.5 pMHC/100 nm² to about 0.9 pMHC/100 nm², or alternatively from about 0.6 pMHC/100 nm² to about 0.8 pMHC/100 nm².

### Linkers

In certain aspects, ubiquitous autoantigen-MHC can be coupled to the nanoparticle core by one or more of covalently, non-covalently, or cross-linked and optionally coupled through a linker. In aspects involving a linker or linkers, the linkers may be the same or different from each other on a single nanoparticle core. In some embodiments, the ubiquitous autoantigen-MHC comprises at least one ubiquitous autoantigen-MHC described herein and a nanoparticle, wherein the nanoparticle is non-liposomal and has metal or metal oxide core; and the at least one ubiquitous autoantigen-MHC is covalently linked to the nanoparticle through a linker comprising polyethylene glycol with a molecular weight of less than 5 kilodaltons (kD). In some embodiments, polyethylene glycol has a molecular weight of less than 1 kD, 2 kD, 3 kD, 4 kD, 5 kD, 6 kD, 7 kD, 8 kD, 9 kD, or 10 kD. In some embodiments, polyethylene glycol is functionalized with maleimide. In some embodiments, polyethylene glycol has a molecular weight of between about 1 kD and about 5 kD, between about 2 kD and about 5 kD, between about 3 kD and about 5 kD. In some embodiments, polyethylene glycol is functionalized with maleimide. In certain embodiments, the end of the linker that is in contact with the solid core is embedded in the solid core. In further aspects, the linker may be less than 5 kD in size, and is optionally polyethylene glycol. The linker can be any of the linkers described in Table 1.

TABLE 1
Exemplary linker molecules

| Types of | | M.W. | Functional | |
|---|---|---|---|---|
| Nanoparticle | PEG linkers | (kD) | group | Structure |
| Gold nano- particle (GNP-C) | Thiol-PEG- carboxyl | 3.5 | Carboxyl (—COOH) | 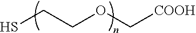 |
| Gold nano- particle (GNP-N) | Thiol-PEG- amine | 3.5 | Amine (—NH₂) | 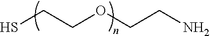 |
| Iron oxide Nanoparticle (SFP-C) | Dopamine- PEG- carboxyl | 3.5 | Carboxyl (—COOH) | 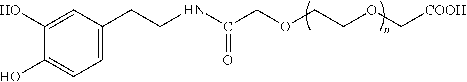 |
| Iron oxide Nanoparticle (SFP-N) | Dopamine- PEG- amine | 3.5 | Amine (—NH₂) | 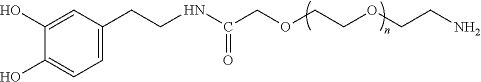 |
| Iron oxide Nanoparticle (SFP-Z) | Dopamine- PEG- azide | 3.5 | Azide (—N₃) | 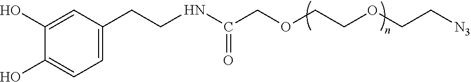 |
| Iron oxide Nanoparticle (SFP-M) | Dopamine- PEG- maleimide | 3.5 | Maleimide | 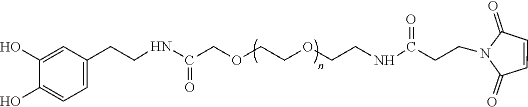 |
| Iron oxide Nanoparticle (SFP-O) | Dopamine- PEG- Ortho- pyridyl disulfide | 3.5 | Orthopyridyl disulfide | 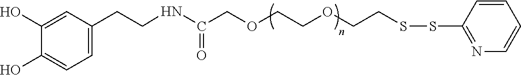 |
| Iron oxide Nano- particle (PF-C) | carboxyl- PEG- carboxyl | 3.5 | Carboxyl (—COOH) | 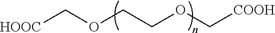 |
| Iron oxide Nano- particle (PF-N) | Methoxy- PEG- amine | 2.0 | Amine (—NH₂) | 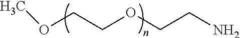 |
| Iron oxide Nano- particle (PF-M) | Methoxy- PEG- maleimide | 2.0 | Maleimide | 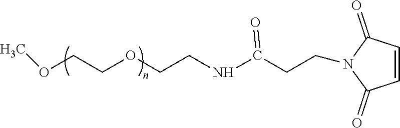 |
| Iron oxide Nano- particle (PF-O) | Methoxy- PEG- Ortho- pyridyl disulfide | 2.0 | Orthopyridyl disulfide | 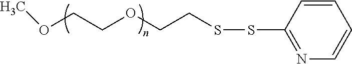 |
| Iron oxide Nano- particle (PF) | PEG | 2.0 | Hydroxyl (—OH) | 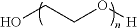 |

In order to couple the substrate or particles of the ubiquitous autoantigen-MHC to the nanoparticle, the following techniques can be applied.

The binding can be generated by chemically modifying the substrate or particle which typically involves the generation of “functional groups” on the surface, said functional groups being capable of binding to an MHC complex, and/or linking the optionally chemically modified surface of the surface or particle with covalently or non-covalently bound so-called “linking molecules,” followed by reacting the MHC or MHC complex with the particles obtained.

The functional groups or the linking molecules bearing them may be selected from amino groups, carbonic acid groups, thiols, thioethers, disulfides, guanidino, hydroxyl groups, amine groups, vicinal diols, aldehydes, alpha-haloacetyl groups, mercury organyles, ester groups, acid halide, acid thioester, acid anhydride, isocyanates, isothiocyanates, sulfonic acid halides, imidoesters, diazoacetates, diazonium salts, 1,2-diketones, phosphoric acids, phosphoric acid esters, sulfonic acids, azolides, imidazoles, indoles, N-maleimides, alpha-beta-unsaturated carbonyl compounds, arylhalogenides or their derivatives.

Non-limiting examples for other linking molecules with higher molecular weights are nucleic acid molecules, polymers, copolymers, polymerizable coupling agents, silica, proteins, and chain-like molecules having a surface with the opposed polarity with respect to the substrate or particle. Nucleic acids can provide a link to affinity molecules containing themselves nucleic acid molecules, though with a complementary sequence with respect to the linking molecule.

In some embodiments, the linking molecule comprises polyethylene glycol. In some embodiments, the linking molecule comprises polyethylene glycol and maleimide. In some embodiments, the polyethylene glycol comprises one or more of a C₁-C₃ alkoxy group, —R¹⁰NHC(O)R—, —R¹⁰C(O)NHR—, —R¹⁰OC(O)R—, —R¹⁰C(O)OR—, wherein each R is independently H or C₁-C₆ alkyl and wherein each R₁₀ is independently a bond or C₁-C₆ alkyl.

pMHCs can be coupled to nanoparticles by a variety of methods, one non-limiting example includes conjugation to NPs produced with PEG linkers carrying distal —NH2 or —COOH groups that can be achieved via the formation of amide bonds in the presence of 1-Ethyl-3-[3-dimethylaminopropyl]carbodiimide hydrochloride (EDC). NPs with —COOH groups are first dissolved in 20 mM MES buffer, pH 5.5. N-hydroxysulfosuccinimide sodium salt (sulpha-NHS, Thermo Scientific, Waltham, MA, final concentration 10 mM) and EDC (Thermo scientific, Waltham, MA, final concentration 1 mM) is added to the NP solution. After 20 min of stirring at room temperature, the NP solution is added drop-wise to the solution containing pMHC monomers dissolved in 20 mM borate buffer (pH 8.2). The mixture is stirred for an additional 4 hr. To conjugate MHCs to NH2-functionalized NPs pMHCs are first dissolved in 20 mM MES buffer, pH 5.5, containing 100 mM NaCl. Sulpha-NHS (10 mM) and EDC (5 mM) are then added to the MHC solution. The activated MHC molecules are then added to the NP solution in 20 mM borate buffer (pH 8.2), and stirred for 4 hr at room temperature.

To conjugate MHC to maleimide-functionalized NPs, pMHCs are first incubated with Tributylphospine (TBP, 1 mM) for 4 hr at room temperature, pMHCs engineered to encode a free carboxyterminal Cys residue are then mixed with NPs in 40 mM phosphate buffer, pH 6.0, containing 2 mM EDTA, 150 mM NaCl, and incubated overnight at room temperature. MHCs of the pMHCs are covalently bound with NPs via the formation of a carbon-sulfide bond between maleimide groups and the Cys residue.

Click chemistry can be used to conjugate pMHC or avidin to NPs functionalized with azide groups. For this reaction, MHC or avidin molecules are first incubated with dibenzocyclooctyl (DBCO, Click Chemistry Tools, Scottsdale, AZ) reagent for 2 hr at room temperature. Free DBCO molecules can be removed by dialysis overnight. MHC- or avidin-DBCO conjugates are then incubated with SFP-Z for 2 hr, resulting in formation of triazole bonds between pMHCs or avidin molecules and NPs.

Unconjugated pMHCs in the different MHC-NP conjugating reactions can be removed by extensive dialysis using methods known in the art. A non-limiting example is dialysis against PBS, pH 7.4, at 40 C though 300 kD molecular weight cut off membranes (Spectrum labs). Alternatively, pMHC-conjugated IONPs can be purified by magnetic separation. The conjugated NPs can be concentrated by ultrafiltration through Amicon Ultra-15 units (100 kD MWCO) and stored in PBS.

The surface of the particle can be chemically modified, for instance by the binding of phosphonic acid derivatives having functional reactive groups. One example of these phosphonic acid or phosphonic acid ester derivates is imino-bis(methylenephosphono) carbonic acid which can be synthesized according to the “Mannich-Moedritzer” reaction. This binding reaction can be performed with a substrate or a particle as directly obtained from the preparation process or after a pre-treatment (for instance with trimethylsilyl bromide). In the first case the phosphoric acid (ester) derivative may for instance displace components of the reaction medium which are still bound to the surface. This displacement can be enhanced at higher temperatures. Trimethylsilyl bromide, on the other hand, is believed to dealkylate alkyl group-containing phosphorous-based complexing agents, thereby creating new binding sites for the phosphonic acid (ester) derivative. The phosphonic acid (ester) derivative, or linking molecules bound thereto, may display the same functional groups as given above. A further example of the surface treatment of the substrate or particle involves heating in a diol such as ethylene glycol. It should be noted that this treatment may be redundant if the synthesis already proceeded in a diol. Under these circumstances the synthesis product directly obtained is likely to show the necessary functional groups. This treatment is, however, applicable to a substrate or a particle that was produced in N- or P-containing complexing agents. If such substrate or particle is subjected to an after-treatment with ethylene glycol, ingredients of the reaction medium (e.g. complexing agent) still binding to the surface can be replaced by the diol and/or can be dealkylated.

It is also possible to replace N-containing complexing agents still bound to the particle surface by primary amine derivatives having a second functional group. The surface of the substrate or particle can also be coated with silica. Silica allows a relatively simple chemical conjugation of organic molecules since silica easily reacts with organic linkers, such as triethoxysilane or chlorosilane. The particle surface may also be coated by homo- or copolymers. Examples for polymerizable coupling agents are: N-(3-aminopropyl)-3-mercaptobenzamidine, 3-(trimethoxysilyl) propylhydrazide and 3-trimethoxysilyl) propylmaleimide. Other non-limiting examples of polymerizable coupling agents are mentioned herein. These coupling agents can be used singly or in combination depending on the type of copolymer to be generated as a coating.

Another surface modification technique that can be used with substrates or particles containing oxidic transition metal compounds is conversion of the oxidic transition metal compounds by chlorine gas or organic chlorination agents to the corresponding oxychlorides. These oxychlorides are capable of reacting with nucleophiles, such as hydroxyl or amino groups as often found in biomolecules. This technique allows generating a direct conjugation with proteins, for instance, via the amino group of lysine side chains. The conjugation with proteins after surface modification with oxychlorides can also be effected by using a bi-functional linker, such as maleimidopropionic acid hydrazide.

For non-covalent linking techniques, chain-type molecules having a polarity or charge opposite to that of the substrate or particle surface are particularly suitable. Examples for linking molecules which can be non-covalently linked to core/shell nanoparticles involve anionic, cationic or zwitter-ionic surfactants, acid or basic proteins, polyamines, polyamides, polysulfone or polycarboxylic acid. The hydrophobic interaction between substrate or particle and amphiphilic reagent having a functional reactive group can generate the necessary link. In particular, chain-type molecules with amphiphilic character, such as phospholipids or derivatised polysaccharides, which can be crosslinked with each other, are useful. The absorption of these molecules on the surface can be achieved by coincubation. The binding between affinity molecule and substrate or particle can also be based on non-covalent, self-organizing bonds. One example thereof involves simple detection probes with biotin as linking molecule and avidin- or streptavidin-coupled molecules.

Protocols for coupling reactions of functional groups to biological molecules can be found in the literature, for instance in “Bioconjugate Techniques” (Greg T. Hermanson, Academic Press 1996). The biological molecule (e.g., MHC molecule or derivative thereof) can be coupled to the linking molecule, covalently or non-covalently, in line with standard procedures of organic chemistry such as oxidation, halogenation, alkylation, acylation, addition, substitution or amidation. These methods for coupling the covalently or non-covalently bound linking molecule can be applied prior to the coupling of the linking molecule to the substrate or particle or thereafter. Further, it is possible, by means of incubation, to effect a direct binding of molecules to correspondingly pre-treated substrate or particles (for instance by trimethylsilyl bromide), which display a modified surface due to this pre-treatment (for instance a higher charge or polar surface).

### Synthesis of Nanoparticles

Nanoparticles may be formed by contacting an aqueous phase containing the pMHC complex and a polymer and a non-aqueous phase followed by evaporation of the non-aqueous phase to cause the coalescence of particles from the aqueous phase as taught in U.S. Pat. No. 4,589,330 or 4,818,542. Certain polymers for such preparations are natural or synthetic copolymers or polymers which include gelatin agar, starch, arabinogalactan, albumin, collagen, polyglycolic acid, polylactic acid, glycolide-L(−) lactide poly(epsilon-caprolactone), poly(epsilon-caprolactone-CO-lactic acid), poly(epsilon-caprolactone-CO-glycolic acid), poly(β-hydroxy butyric acid), poly(ethylene oxide), polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly, certain polymers are polyesters, such as polyglycolic acid, polylactic acid, glycolide-L(−) lactide poly(epsilon-caprolactone), poly(epsilon-caprolactone-CO-lactic acid), and poly(epsilon-caprolactone-CO-glycolic acid). Solvents useful for dissolving the polymer include: water, hexafluoroisopropanol, methylenechloride, tetrahydrofuran, hexane, benzene, or hexafluoroacetone sesquihydrate.

Gold nanoparticles (GNPs) are synthesized using chemical reduction of gold chloride with sodium citrate as described in Perrault, S.D. et al. (2009) Nano Lett 9:1909-1915. Briefly, 2 mL of 1% of HAuCl4 (Sigma Aldrich) is added to 100 mL H2O under vigorous stirring and the solution is heated in an oil bath. Six (for 14 nm GNPs) or two mL (for 40 nm GNPs) of 1% Na Citrate is added to the boiling HAuCl4 solution, which is stirred for an additional 10 min and then is cooled down to room temperature. GNPs are stabilized by the addition of 1 μMol of thiol-PEG linkers (Nanocs, MA) functionalized with —COOH or —NH2 groups as acceptors of MHC. PEGylated GNPs are washed with water to remove free thiol-PEG, concentrated and stored in water for further analysis. NP density is determined via spectrophotometry and calculated according to Beer's law.

The SFP series of iron oxide NPs (SFP IONPs) can be produced by thermal decomposition of iron acetate in organic solvents in the presence of surfactants, then rendered solvent in aqueous buffers by pegylation (Xie, J. et al. (2007) Adv Mater 19:3163; Xie, J. et al. (2006) Pure Appl. Chem. 78:1003-1014; Xu, C. et al. (2007) Polymer International 56:821-826). Briefly, 2 mMol Fe(acac)₃ (Sigma Aldrich, Oakville, ON) are dissolved in a mixture of 10 mL benzyl ether and oleylamine and heated to 100° C. for 1 hr followed by 300° C. for 2 hr with reflux under the protection of a nitrogen blanket. Synthesized NPs are precipitated by addition of ethanol and resuspended in hexane. For pegylation of the IONPs, 100 mg of different 3.5 kD DPA-PEG linkers (Jenkem Tech USA) are dissolved in a mixture of CHCl₃ and HCON(CH₃)₂ (dimethylformamide (DMF)). The NP solution (20 mg Fe) is then added to the DPA-PEG solution and stirred for 4 hr at room temperature. Pegylated SFP NPs are precipitated overnight by addition of hexane and then resuspended in water. Trace amounts of aggregates are removed by high-speed centrifugation (20,000×g, 30 min), and the monodisperse SFP NPs are stored in water for further characterization and pMHC conjugation. The concentration of iron in IONP products is determined by spectrophotometry at A410 in 2N HCL. Based on the molecular structure and diameter of SFP NPs (Fe₃O₄; 8+1 nm diameter) (Xie, J. et al. (2007) Adv Mater 19:3163; Xie, J. et al. (2006) Pure Appl. Chem. 78:1003-1014), SFP solutions containing 1 mg of iron are estimated to contain 5×10¹⁴ NPs.

The nanoparticles can also be made by thermally decomposing or heating a nanoparticle precursor. In one embodiment, the nanoparticle is a metal or a metal oxide nanoparticle. In one embodiment, the nanoparticle is an iron oxide nanoparticle. In one embodiment, the nanoparticle is a gold nanoparticle. In one embodiment, provided herein are the nanoparticles prepared in accordance with the present technology. In one embodiment, provided herein is a method of making iron oxide nanoparticles comprising a thermal decomposition reaction of iron acetyl acetonate. In one embodiment, the iron oxide nanoparticle obtained is water-soluble. In one aspect, the iron oxide nanoparticle is suitable for protein conjugation. In one embodiment, the method comprises a single-step thermal decomposition reaction.

In one aspect, the thermal decomposition occurs in the presence of functionalized PEG molecules. Certain non-limiting examples of functionalized PEG linkers are shown in Table 1.

In one aspect, the thermal decomposition comprises heating iron acetyl acetonate. In one embodiment, the thermal decomposition comprises heating iron acetyl acetonate in the presence of functionalized PEG molecules. In one embodiment, the thermal decomposition comprises heating iron acetyl acetonate in the presence of benzyl ether and functionalized PEG molecules] Without being bound by theory, in one embodiment, functionalized PEG molecules are used as reducing reagents and as surfactants. The method of making nanoparticles provided herein simplifies and improves conventional methods, which use surfactants that are difficult to be displaced, or are not displaced to completion, by PEG molecules to render the particles water-soluble. Conventionally, surfactants can be expensive (e.g., phospholipids) or toxic (e.g., Oleic acid or oleilamine). In another aspect, without being bound by theory, the method of making nanoparticles obviates the need to use conventional surfactants, thereby achieving a high degree of molecular purity and water solubility.

In one embodiment, the thermal decomposition involves iron acetyl acetonate and benzyl ether and in the absence of conventional surfactants other than those employed herein.

In one embodiment, the temperature for the thermal decomposition is about 80° C. to about 300° C., or about 80° C. to about 200° C., or about 80° C. to about 150° C., or about 100° C. to about 250° C., or about 100° C. to about 200° C., or about 150° C. to about 250° C., or about 150° C. to about 250° C. In one embodiment, the thermal decomposition occurs at about 1 to about 2 hours of time.

In one embodiment, the method of making the iron oxide nanoparticles comprises a purification step, such as by using Miltenyi Biotec LS magnet column.

In one embodiment, the nanoparticles are stable at about 4° C. in phosphate buffered saline (PBS) without any detectable degradation or aggregation. In one embodiment, the nanoparticles are stable for at least 6 months.

In one aspect, provided herein is a method of making nanoparticle complexes comprising contacting pMHC with iron oxide nanoparticles provided herein. Without being bound by theory, pMHC encodes a cysteine at its carboxyterminal end, which can react with the maleimide group in functionalized PEG at about pH 6.2 to about pH 6.5 for about 12 to about 14 hours.

In one aspect, the method of making nanoparticle complexes comprises a purification step, such as by using Miltenyi Biotec LS magnet column.

### Regulatory Immune Cell Types

The uaMHC-NP complexes of the current disclosure reprogram or differentiate autoreactive T cells into T regulatory or TR1 cells. In certain embodiments, the TRI cells express IL-10. In certain embodiments, the TRI cells secrete IL-10. In certain embodiments, the TRI cells express CD49b. In certain embodiments, the TRI cells express LAG-3. T-cells that have these phenotypic characteristics are useful to treat inflammatory or autoimmune conditions of individuals. In certain embodiments, the uaMHC-NP complexes are useful in a method to reprogram or differentiate autoreactive T cells into TR1 cells in an individual after administration. This method generates TR1 cells in an antigen specific way.

The ubiquitous autoantigen-MHCs of the current disclosure are useful for generating B regulatory cells. In certain embodiments, the ubiquitous autoantigen-MHCs of the current disclosure are deployed in a method to generate B-cells expressing high levels of CD1d, CD5, and/or the secretion of IL-10. B-regs are also identified by expression of Tim-1. In certain embodiments, the uaMHC-NP complexes are useful in a method to induce B regulatory cells in an individual after administration. This method generates B regulatory cells in an antigen specific way.

### Pharmaceutical Compositions and Administration

Provided herein are pharmaceutical compositions of ubiquitous autoantigen-MHC-NPs useful for the treatment and prevention of disease. The compositions comprise, or alternatively consist essentially of, or yet further consist of, a nanoparticle complex as described herein and a carrier.

Compositions of the disclosure may be conventionally administered parenterally, by injection, for example, intravenously, subcutaneously, or intramuscularly. Additional formulations which are suitable for other modes of administration include oral formulations. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%. The preparation of an aqueous composition that contains an antigen-MHC-nanoparticle complex that modifies the subject's immune condition will be known to those of skill in the art in light of the present disclosure. In one embodiment, the ubiquitous autoantigen-MHC-nanoparticle complex is administered systemically. In specific embodiments, the ubiquitous autoantigen-MHC-NP complex or the compositions comprising a plurality of ubiquitous autoantigen-MHC-N complexes can be administered intravenously.

Typically, the ubiquitous autoantigen-MHC-NPs, described herein, are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner. However, suitable dosage ranges are of the order of ten to several hundred nanograms or micrograms of antigen/MHC/nanoparticle complex per administration. Suitable regimes for initial administration and boosters are also variable, but are typified by an initial administration followed by subsequent administrations.

The manner of application may be varied widely. Any of the conventional methods for administration of a vaccine are applicable. These are believed to include oral application on a solid physiologically acceptable base or in a physiologically acceptable dispersion, parenterally, by injection and the like. The dosage of the antigen/MHC/nanoparticle complex will depend on the route of administration and will vary according to the size and health of the subject. The ubiquitous autoantigen-MHC-NPs can be administered by any suitable route including intravenous, subcutaneous, intradermal, intramuscular, rectally, or intraperitoneally. In certain embodiments, autoantigen-MHC-NPs are administered parenterally. In certain embodiments, autoantigen-MHC-NPs are administered intravenously. In certain embodiments, autoantigen-MHC-NPs are administered subcutaneously.

In many instances, it will be desirable to have multiple administrations of a ubiquitous autoantigen-MHC-NP, about, at least about, or at most about 3, 4, 5, 6, 7, 8, 9, 10 or more administrations. The administrations will normally range from 1, 2, 3, 4, 5, 6, or 7 day to twelve week intervals, more usually from one to two week intervals. Periodic boosters at intervals of every other day, twice a week, weekly, biweekly, monthly, or 0.1, 0.2, 0.3, 0.4, 0.5, 1, 2, 3, 4 or 5 years, usually two years, will be desirable to maintain the condition of the immune system. The course of the administrations may be followed by assays for autoreactive immune responses, cognate TR1 cells, and T cell activity.

In certain aspects, a single dose of the ubiquitous autoantigen-MHC-NP without including the nanoparticle core and any bioabsorbable/biocompatible outer layer comprises about 0.001 mg/kg to about 2.0 mg/kg, or about 0.001 mg/kg to about 1.5 mg/kg, or about 0.001 mg/kg to about 1.4 mg/kg, or about 0.001 mg/kg to about 1.3 mg/kg, or about 0.001 mg/kg to about 1.2 mg/kg, or about 0.001 mg/kg to about 1.1 mg/kg, or about 0.001 mg/kg to about 1.0 mg/kg. In some embodiments, the single dose comprises from about 0.004 mg/kg to about 1.014 mg/kg, or from about 0.02 mg/kg to about 0.811 mg/kg, or from about 0.041 mg/kg to about 0.608 mg/kg, or from about 0.061 mg/kg to about 0.507 mg/kg, or from about 0.081 mg/kg to about 0.405 mg/kg, or from about 0.121 mg/kg to about 0.324 mg/kg, or from about 0.162 mg/kg to about 0.243 mg/kg. In some embodiments, the single dose comprises from about 0.004 mg/kg to about 1.015 mg/kg, or from about 0.004 mg/kg to about 1.0 mg/kg, or from about 0.004 mg/kg to about 0.9 mg/kg, or from about 0.004 mg/kg to about 0.8 mg/kg, or from about 0.004 mg/kg to about 0.7 mg/kg, or from about 0.004 mg/kg to about 0.6 mg/kg, or from about 0.004 mg/kg to about 0.5 mg/kg, or from about 0.004 mg/kg to about 0.4 mg/kg, or from about 0.004 mg/kg to about 0.3 mg/kg, or from about 0.004 mg/kg to about 0.2 mg/kg, or from about 0.004 mg/kg to about 0.1 mg/kg. Herein, mg/kg refers to milligrams of ubiquitous autoantigen-MHC or ubiquitous autoantigen without considering the MHC component, administered per kg of subject body mass.

### Autoimmune and Inflammatory Diseases

The ubiquitous autoantigen-MHCs of the current disclosure are useful for treating an autoimmune or inflammatory disorder. Autoimmune or inflammatory disorders include diseases or disorders arising from and directed against an individual's own tissues or organs or a manifestation thereof or a condition resulting therefrom. In one embodiment, it refers to a condition that results from, or is aggravated by, the production of T cells that are reactive with normal body tissues and antigens. In one embodiment, it refers to a condition that results from, or is aggravated by, the production by antibodies that are reactive with normal body tissues and antigens. Examples of autoimmune or inflammatory disorders include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis (such as plaque psoriasis, gutatte psoriasis, pustular psoriasis, and psoriasis of the nails), atopy (including atopic diseases such as hay fever and Job's syndrome), dermatitis (including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis), x-linked hyper IgM syndrome, allergic intraocular inflammatory diseases, urticaria (such as chronic allergic urticaria and chronic idiopathic urticaria, including chronic autoimmune urticaria), myositis, polymyositis/dermatomyositis, juvenile dermatomyositis, toxic epidermal necrolysis, scleroderma (including systemic scleroderma), sclerosis (such as systemic sclerosis; multiple sclerosis (MS) such as spino-optical MS, primary progressive MS (PPMS), and relapsing-remitting MS (RRMS); progressive systemic sclerosis, atherosclerosis, arteriosclerosis, sclerosis disseminata, and ataxic sclerosis), neuromyelitis optica spectrum disorder (NMO, also known as Devic's Disease or Devic's Syndrome), inflammatory bowel disease (IBD) (for example, Crohn's disease; autoimmune-mediated gastrointestinal diseases; colitis such as ulcerative colitis, colitis ulcerosa, microscopic colitis, collagenous colitis, colitis polyposa, necrotizing enterocolitis, and transmural colitis; and autoimmune inflammatory bowel disease), bowel inflammation, pyoderma gangrenosum, erythema nodosum, primary sclerosing cholangitis, respiratory distress syndrome (including adult or acute respiratory distress syndrome (ARDS)), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis (such as anterior uveitis, acute anterior uveitis, granulomatous uveitis, nongranulomatous uveitis, phacoantigenic uveitis, posterior uveitis, or autoimmune uveitis), glomerulonephritis (GN) with and without nephrotic syndrome (such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, or proliferative nephritis), autoimmune polyglandular endocrine failure, balanitis including balanitis circumscripta plasmacellularis, balanoposthitis, erythema annulare centrifugum, erythema dyschromicum perstans, erythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema (including allergic or atopic eczema, asteatotic eczema, dyshidrotic eczema, and vesicular palmoplantar eczema), asthma (such as asthma bronchiale, bronchial asthma, and auto-immune asthma), conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus (including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus), Type I diabetes, Type II diabetes, and latent autoimmune diabetes in adults (or Type 1.5 diabetes). Also contemplated are immune responses associated with acute and delayed hypersensitivity mediated by cytokines and T-lymphocytes, sarcoidosis, granulomatosis (including lymphomatoid granulomatosis, Wegener's granulomatosis, or agranulocytosis), vasculitides (including vasculitis, large-vessel vasculitis (including polymyalgia rheumatica and giant cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis (such as Churg-Strauss vasculitis or syndrome (CSS) and ANCA-associated small-vessel vasculitis)), temporal arteritis, aplastic anemia, autoimmune aplastic anemia, Coombs positive anemia, Diamond Blackfan anemia, hemolytic anemia, immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome (such as those secondary to septicemia, trauma, or hemorrhage), antigen-antibody complex-mediated diseases, anti-glomerular basement membrane disease, anti-phospholipid antibody syndrome, anti-phospholipid syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenia purpura (ITP) including chronic or acute ITP, acquired thrombocytopeniarpura, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases (including thyroiditis (such as autoimmune thyroiditis, Hashimoto's disease, chronic thyroiditis (Hashimoto's thyroiditis), or subacute thyroiditis), autoimmune thyroid disease, idiopathic hypothyroidism, or Grave's disease), polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyly, and telangiectasia), male and female autoimmune infertility (e.g., due to anti-spermatozoan antibodies) mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuchs' cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritis, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, emphysema, alopecia areata, adipose tissue inflammation/diabetes type II, obesity associated adipose tissue inflammation/insulin resistance, and endometriosis.

In certain embodiments, the autoimmune disease or inflammatory disorder may include, but is not limited to, diabetes mellitus Type I and Type II, pre-diabetes, transplantation rejection, multiple sclerosis, a multiple-sclerosis related disorder, premature ovarian failure, scleroderma, Sjogren's disease/syndrome, lupus, vitiligo, alopecia (baldness), polyglandular failure, Grave's disease, hypothyroidism, polymyositis, pemphigus, Crohn's disease, colitis, autoimmune hepatitis, hypopituitarism, myocarditis, Addison's disease, autoimmune skin diseases, uveitis, pernicious anemia, hypoparathyroidism, and/or rheumatoid arthritis. Other indications of interest include, but are not limited to, asthma, allergic asthma, primary biliary cirrhosis, cirrhosis, Neuromyelitis Optica Spectrum Disorder (Devic's disease, opticospinal multiple sclerosis (OSMS)), Pemphigus vulgaris, inflammatory bowel disease (IBD), arthritis, Rheumatoid arthritis, systemic lupus erythematosus (SLE), Celiac disease, psoriasis, autoimmune cardiomyopathy, idiopathic dilated cardiomyopathy (IDCM), a Myasthenia Gravis, Uveitis, Ankylosing Spondylitis, Immune Mediated Myopathies, prostate cancer, anti-phospholipid syndrome (ANCA+), atherosclerosis, dermatomyositis, chronic obstructive pulmonary disease (COPD), emphysema, spinal cord injury, traumatic injury, a tobacco-induced lung destruction, ANCA-associated vasculitis, psoriasis, sclerosing cholangitis, primary sclerosing cholangitis, and diseases of the central and peripheral nervous systems.

In certain embodiments, the autoimmune disease or inflammatory disorder may include, but is not limited to, type I diabetes, multiple sclerosis, Celiac Disease, primary biliary cirrhosis, pemphigus, pemphigus foliaceus, pemphigus vulgaris, neuromyelitis optica spectrum disorder, arthritis (including rheumatoid arthritis), allergic asthma, inflammatory bowel disease (including Crohn's disease and ulcerative colitis), systemic lupus erythematosus, atherosclerosis, chronic obstructive pulmonary disease, emphysema, psoriasis, autoimmune hepatitis, uveitis, Sjogren's Syndrome, scleroderma, anti-phospholipid syndrome, ANCA-associated vasculitis, and Stiff Man Syndrome. In a further aspect, the disease-relevant antigen is a tumor- or cancer-relevant antigen.

In certain embodiments, the autoimmune disease or inflammatory disorder may include a liver autoimmune disorder selected from primary biliary cirrhosis, autoimmune hepatitis, and primary sclerosing cholangitis. In certain embodiments, the uaMHC-NP complex is used in a method of treating primary biliary cirrhosis, autoimmune hepatitis, and/or primary sclerosing cholangitis. In certain embodiments, the method of treating primary biliary cirrhosis, autoimmune hepatitis, and/or primary sclerosing cholangitis comprises administering a uaMHC-NP complex, wherein the ubiquitous autoantigen is a polypeptide derived from any one or more of pyruvate dehydrogenase complex-E2 component (PDC-E2), cytochrome P450 2D6 (CYP2D6), soluble liver antigen (SLA), actin (ACTB), formimidoyltransferase-cyclodeaminase (FTCD), and myeloperoxidase (MPO). In certain embodiments, the method of treating primary biliary cirrhosis comprises administering a uaMHC-NP complex, wherein the ubiquitous autoantigen is a polypeptide derived from any one or more of cytochrome P450 2D6 (CYP2D6), soluble liver antigen (SLA), actin (ACTB), formimidoyltransferase-cyclodeaminase (FTCD), and myeloperoxidase (MPO). In certain embodiments, the method of treating autoimmune hepatitis comprises administering a uaMHC-NP complex, wherein the ubiquitous autoantigen is a polypeptide derived from any one or more of pyruvate dehydrogenase complex-E2 component (PDC-E2), soluble liver antigen (SLA), actin (ACTB), formimidoyltransferase-cyclodeaminase (FTCD), and myeloperoxidase (MPO). In certain embodiments, the method of treating primary sclerosing cholangitis comprises administering a uaMHC-NP complex, wherein the ubiquitous autoantigen is a polypeptide derived from any one or more of pyruvate dehydrogenase complex-E2 component (PDC-E2), cytochrome P450 2D6 (CYP2D6), soluble liver antigen (SLA), actin (ACTB), and myeloperoxidase (MPO).

In certain embodiments, the autoimmune disease or inflammatory disorder may include multiple sclerosis. In certain embodiments, the autoimmune disease or inflammatory disorder may include relapsing-remitting multiple sclerosis. In certain embodiments, the uaMHC-NP complex is used in a method of treating multiple sclerosis. In certain embodiments, the method of treating multiple sclerosis comprises administering a uaMHC-NP complex, wherein the ubiquitous autoantigen is a polypeptide derived from any one or more of pyruvate dehydrogenase complex-E2 component (PDC-E2), cytochrome P450 2D6 (CYP2D6), soluble liver antigen (SLA), actin (ACTB), formimidoyltransferase-cyclodeaminase (FTCD), and myeloperoxidase (MPO).

### Pharmaceutically Acceptable Stabilizers, Excipients, and Diluents

In certain embodiments, the uaMHC-NP complexes of the current disclosure are included in a pharmaceutical composition comprising one or more pharmaceutically acceptable stabilizers excipients, carriers, and diluents. In certain embodiments, the uaMHC-NP complexes of the current disclosure are administered suspended in a sterile solution. In certain embodiments, the solution comprises 0.9% NaCl. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EGTA or EGTA. In certain embodiments, the uaMHC-NP complexes of the current disclosure are shipped/stored lyophilized and reconstituted before administration. In certain embodiments, the lyophilized uaMHC-NP complexes formulations comprise a bulking agent such as mannitol, sorbitol, sucrose, trehalose, or dextran 40. The lyophilized formulation can be contained in a vial comprised of glass. The uaMHC-NP complexes, when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0. In certain embodiments, the uaMHC-NP complexes can be formulated for intravenous injection. In certain embodiments, uaMHC-NP complexes can be formulated for oral ingestion. In certain embodiments, uaMHC-NP complexes can be formulated for parenteral, intramuscular, or intra tissue injection. In certain embodiments, uaMHC-NP complexes can be formulated and/or administered without any immunological adjuvant or other compound or polypeptide intended to increase or decrease an immune response.

### EXAMPLES

The following illustrative examples are representative of embodiments of the compositions and methods described herein and are not meant to be limiting in any way.

### Example 1-TRI Like CD4+ T-Cell Formation and Expansion by PBC-Relevant pMHC Class II-NPs

NOD.c3c4 mice, which carry anti-diabetogenic B6-derived chromosome 3 and 4 regions, spontaneously develop a form of autoimmune biliary ductal disease that resembles human PBC. See Irie, J., et al. J. Exp. Med. 203, 1209-1219. Like >90% of patients, these mice develop pathogenic T- and B-cell responses against the E2 and E3BP components of the pyruvate dehydrogenase (PDC) complex. See Kita, H. et al. J. Clin. Invest. 109, 1231-1240. In NOD.c3c4 mice as well as in humans, these autoimmune responses promote the destruction of biliary epithelial cells, leading to cholestasis, small bile duct proliferation, and finally liver failure.

To design PBC-relevant pMHC class II-based nanomedicines, we searched for 15mer peptides in murine PDC-E2 capable of binding to the NOD/NOD.c3c4 mouse MHC class II molecule (I-A^{g7}) in silico. I-A^{g7}-based pMHCs encoding two such epitopes (PDC-E2₁₆₆₋₁₈₁ and PDC-E2₈₂₋₉₆) were chosen for experimentation. The TID-relevant I-A^{g7}-binding BDC2.5 mimotope was used as a negative control. These complexes were produced in lentiviral-transduced Chinese hamster ovary (CHO) cells, purified by sequential nickel and streptag affinity chromatography, and covalently coated via a free carboxyterminal cysteine onto iron-oxide nanoparticles produced by thermal decomposition of Iron (III) acetylacetonate (Fe (acac) 3) in the presence of maleimide-functionalized polyethylene glycol, as described in Singha, S. et al. Nature Nanotechnology 12, 701-710.

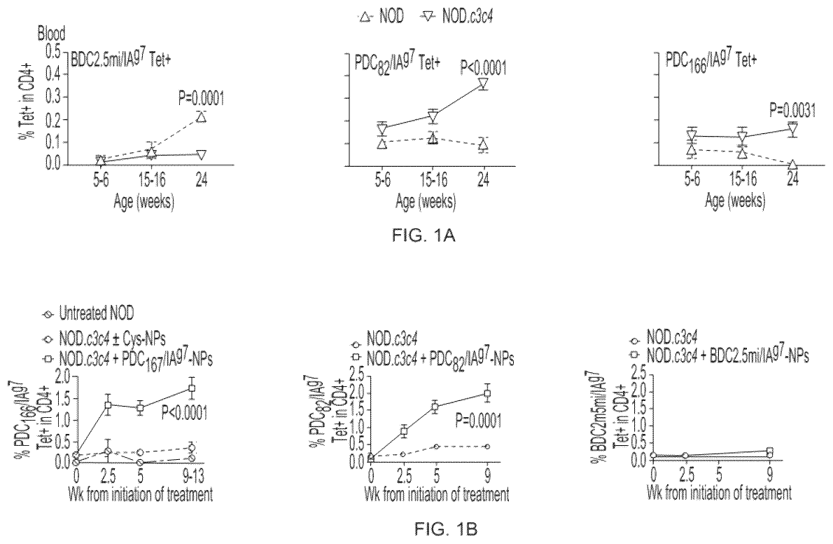

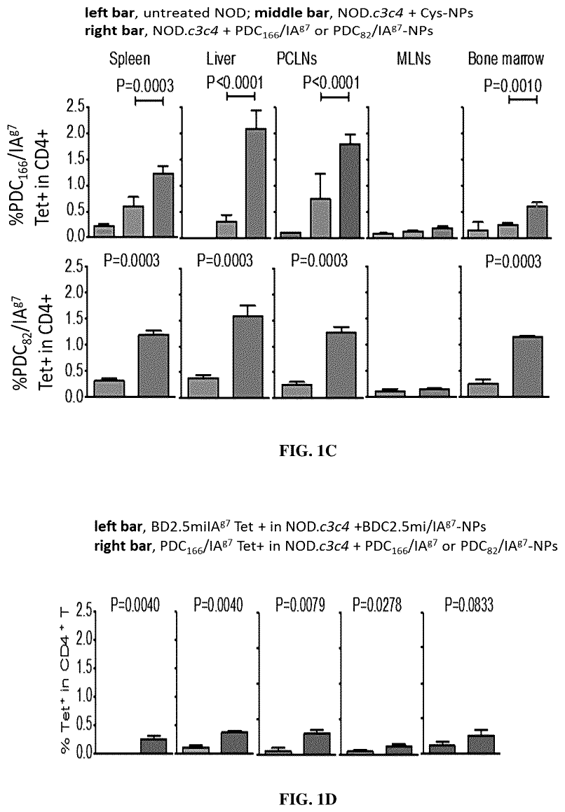

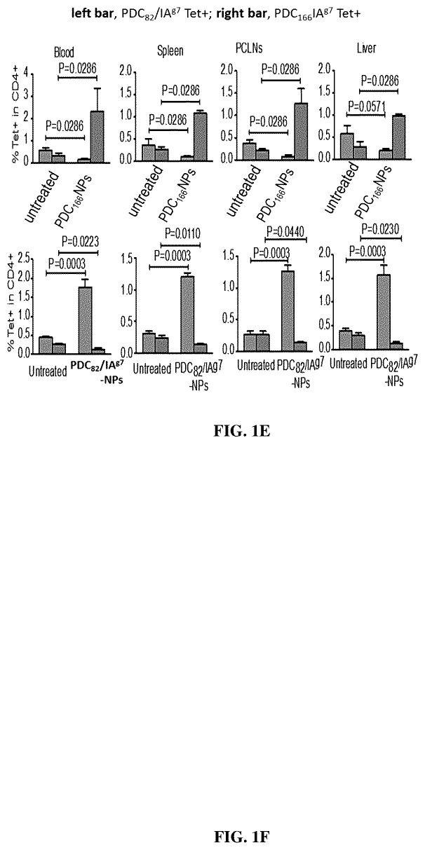

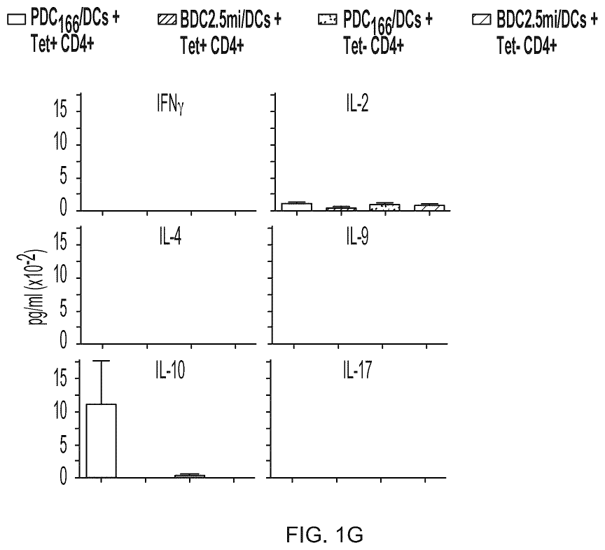

pMHC tetramer staining studies demonstrated that NOD.c3c4 (but not NOD) mice harbor increasing levels of both the PDC-E2₁₆₆₋₁₈₁/IA^{g7} and PDC-E2₈₂₋₉₆/IA^{g7}-reactive T-cell subsets with age as shown in FIG. 1A (upside-down triangles, middle and right panels). In contrast, and unlike NOD mice, NOD.c3c4 mice contain negligible levels of the TID-relevant BDC2.5 mi/IA^{g7}-reactive subset as shown in FIG. 1A (triangles, middle and right panels), an outcome that is consistent with the PBC vs TID proclivity of these two strains. Thus, progression of liver autoimmunity in NOD.c3c4 mice is accompanied by increases in the size and/or circulating activity of PDC-E2-specific CD4+ T-cells.

To ascertain if PBC-relevant pMHC class II-NPs could trigger the formation and expansion of PDC-E2-specific TR1-like CD4+ T-cells in NOD.c3c4 mice, we first treated 15 week-old NOD.c3c4 mice (when the disease is well established) with NPs displaying the PDC-E2₁₆₆₋₁₈₁/IA^{g7} pMHC or control NPs (Cys-NPs) (twice a week intravenous for up to 13.5 weeks). Treatment triggered a rapid increase (within 2.5 weeks) in the peripheral frequency of circulating PDC-E2₁₆₆₋₁₈₁/IA^{g7} tetramer+CD4+ T-cells as compared to mice treated with bare NPs or to untreated NOD mice as shown in FIG. 1B (squares, left and middle panels). Studies of mice at the end of follow up confirmed that the PDC-E2₁₆₆₋₁₈₁/IA^{g7}-NP-induced expansion of cognate CD4+ T-cells was systemic, with increased frequencies in spleen, bone marrow, liver and liver-draining (portal and celiac lymph nodes), but not non-draining lymph nodes (vs. mesenteric lymph nodes (MLN)) as shown in FIG. 1C.

In contrast, treatment of NOD.c3c4 with NPs coated with the TID-relevant BDC2.5/I-A^{g7} pMHCs did not trigger TR1 cell formation or expansion (FIG. 1B right panel and FIG. 1D). This result is consistent with these nanomedicines exclusively operating on autoantigen-experienced T-cells; NOD.c3c4 mice do not develop islet inflammation and therefore are not expected to harbor antigen-activated BDC2.5 mi/IA^{g7}-autoreactive CD4+ T-cells.

Experiments in additional cohorts of mice demonstrated that the PDC-E2₁₆₆₋₁₈₁/IA^{g7}-NP-induced expansion of cognate CD4+ T-cells was peptide-specific, without any detectable expansion of PDC-E2₈₂₋₉₆/I-A^{g7}-reactive CD4+ T-cells (FIG. 1E, top row). In fact, expansion of PDC-E2₁₆₆₋₁₈₁/IA^{g7}-specific CD4+ T-cells in these mice was accompanied by significant reductions in the frequency of PDC-E2₈₂₋₉₆/I-A^{g7}-reactive CD4+ T-cells in all organs examined, as compared to the levels detected in age-matched untreated mice (FIG. 1E, top row). This suggests that the PDC-E2₁₆₆₋₁₈₁/IA^{g7}-specific CD4+ T-cell subset somehow inhibited the proliferation of its PDC-E2₈₂₋₉₆/I-A^{g7}-reactive counterpart in response to endogenous autoantigen exposure.

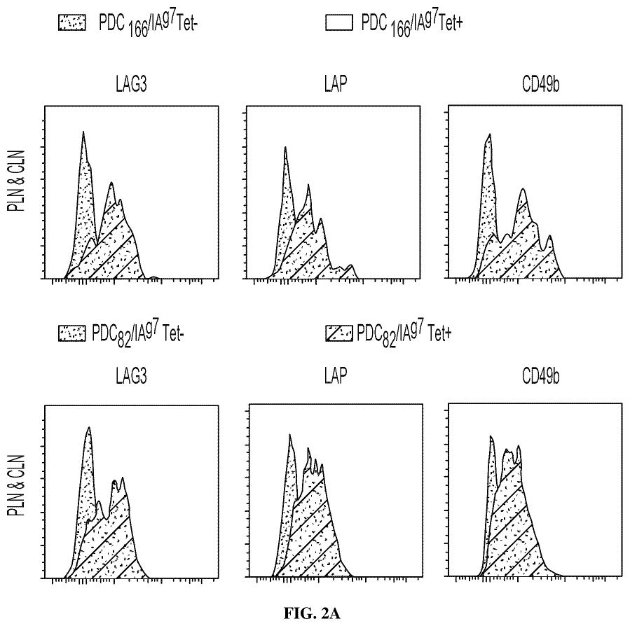

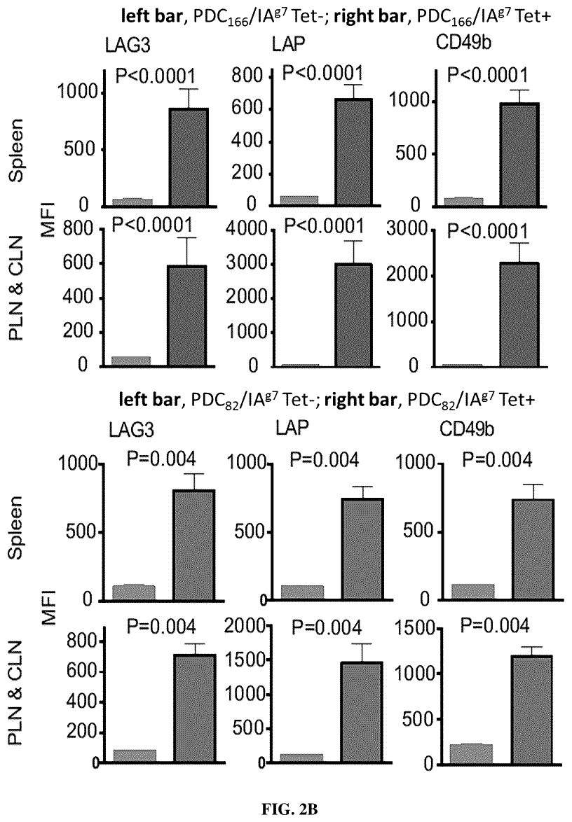

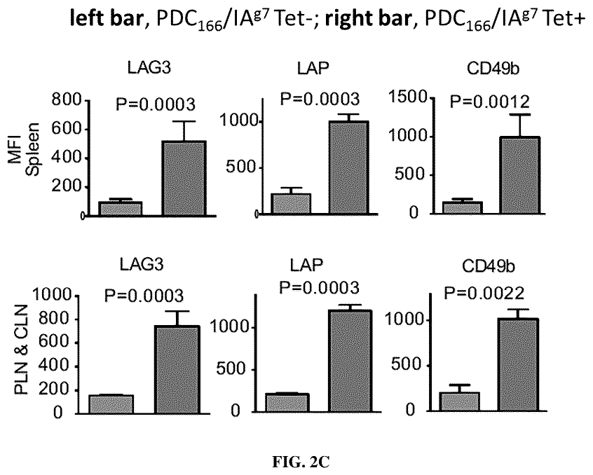

As expected, the PDC-E2₁₆₆₋₁₈₁/IA^{g7} tetramer+CD4+ T-cells that expanded in these mice expressed the TR1 cell markers LAG-3, CD49b and LAP as shown in FIG. 1F and FIGS. 2A and 2B. Furthermore, unlike their tetramer-negative counterparts, the splenic tetramer+CD4+ T-cells of these mice produced the TR1 cytokine IL-10 but not IFNγ, IL-2, IL-4, IL-9 or IL-17 in response to PDC-E2₁₆₆₋₁₈₁ (but not BDC2.5) peptide-pulsed bone marrow-derived DCs (FIG. 1G). Similar results were obtained in mice treated with NPs displaying the second PDC-E2-based pMHC (PDC-E2₈₂₋₉₆/I-A^{g7}), in which there was a significant expansion of cognate PDC-E2₈₂₋₉₆/I-A^{g7}-reactive TR1-like CD4+ T-cells and significant reductions in the frequency of PDC-E2₁₆₆₋₁₈₁/IA^{g7}-reactive CD4+ T-cells as shown in FIGS. 1B (center) and 1E (bottom) and FIGS. 2A and 2B, indicating that the above outcome is not a peculiarity of any particular epitope on PDC-E2.

Together, the above data demonstrate that PDC-E2 peptide/IA^{g7}-NPs efficiently trigger the formation and expansion of cognate TR1-like CD4+ T-cells in NOD.c3c4 mice, as described previously for TID-, EAE- and CIA-relevant pMHC class II-NPs in the corresponding disease models.

### Example 2—Reversal of Established PBC by Disease-Relevant pMHC Class II-NPs

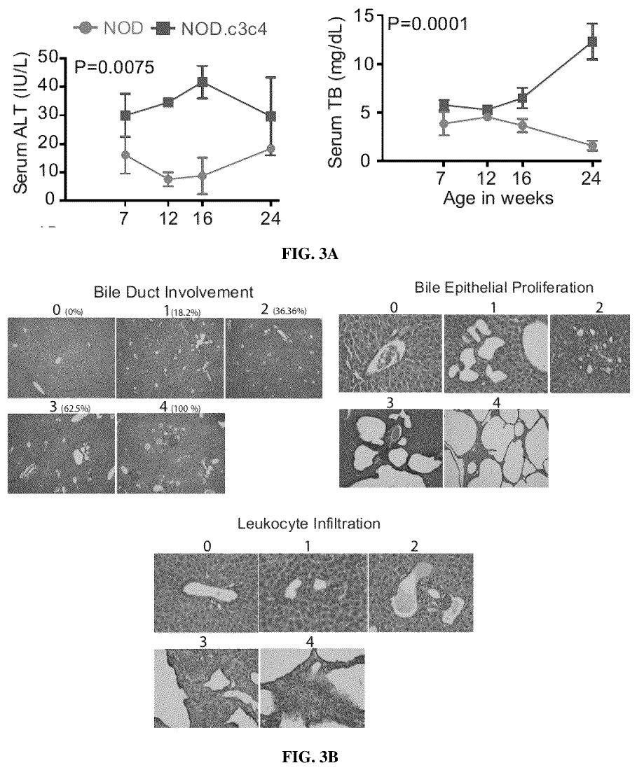

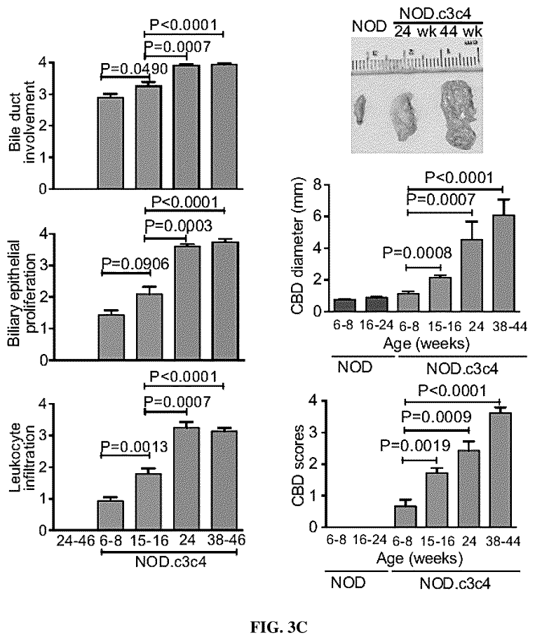

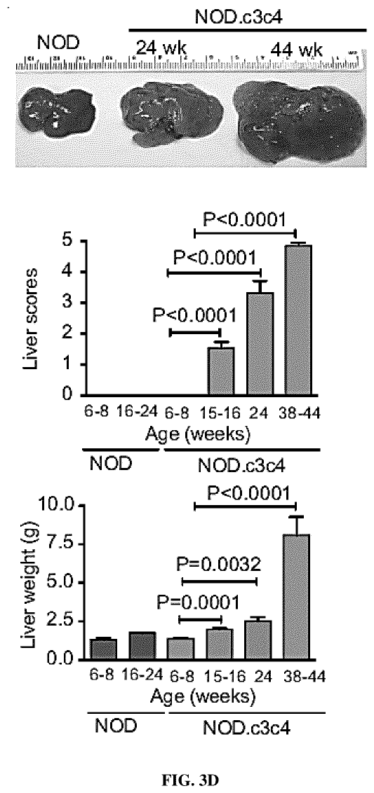

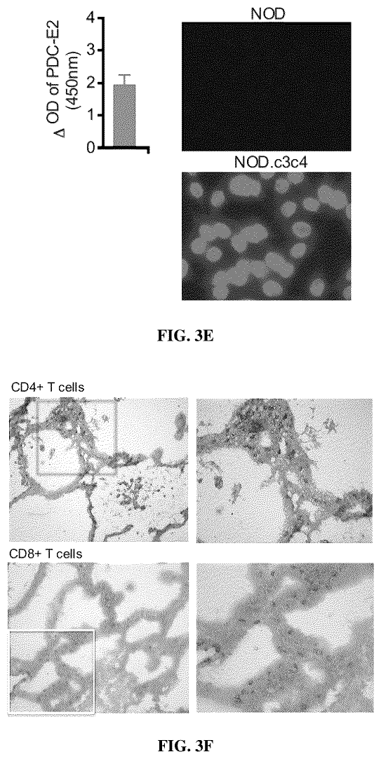

When compared to age-matched NOD mice, 6-8 week-old NOD.c3c4 mice begin to display elevated levels of serum alanine aminotransferase (ALT), microscopic biliary epithelial proliferation, biliary track leukocyte infiltration, massive bile duct involvement (near maximum number of portal triads affected) and macroscopic enlargement of the common biliary duct (CBD) (FIGS. 3A and 3C). FIG. 3B shows an exemplary scoring matrix to quantify microscopic analysis. By ~15-16 weeks these signs worsen and the mice begin to display increased total serum bilirubin (TB) levels (FIG. 3A) high titers of anti-mitochondrial/PDC-E2-specific autoantibodies (absent in NOD mice; FIG. 3E) and macroscopic signs of liver disease (bile cysts) (FIG. 3D). The severity of all these signs of disease peaks at ~24 weeks of age (FIGS. 3A-3D), coinciding with massive infiltration of the biliary epithelium by CD4+ and CD8+ T-cells (FIG. 3F), high titers of anti-nuclear autoantibodies (ANAs) (FIG. 3E) and a nearly three-fold increase in liver weight (FIG. 3D).

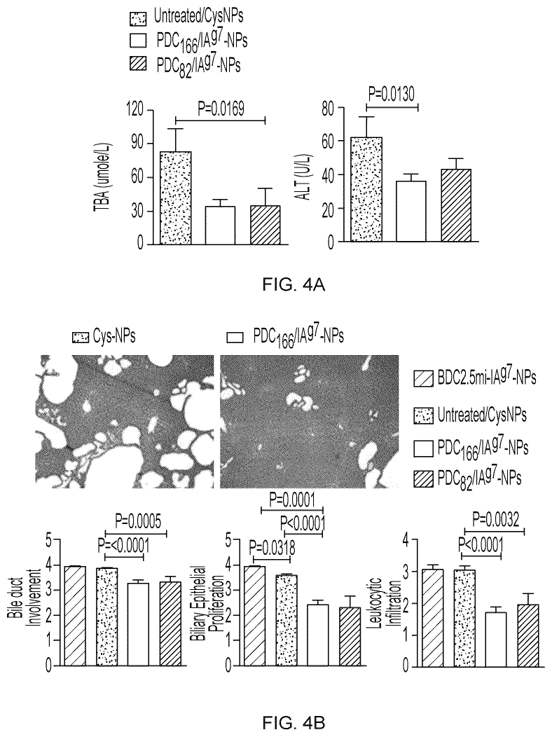

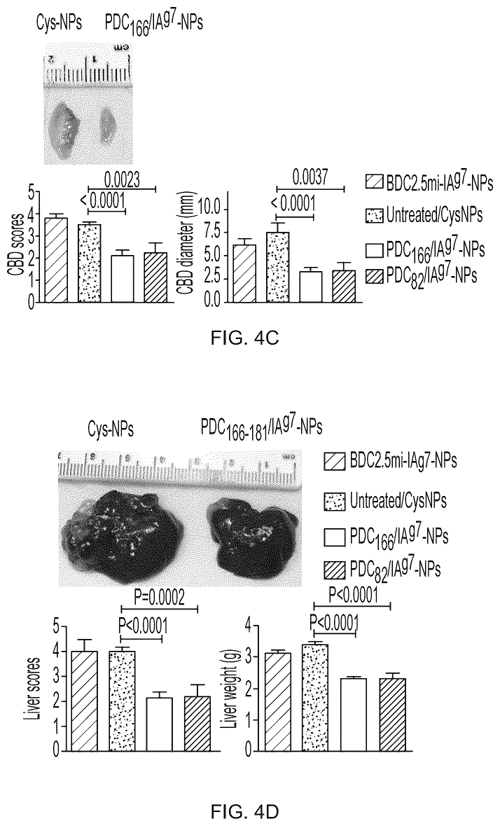

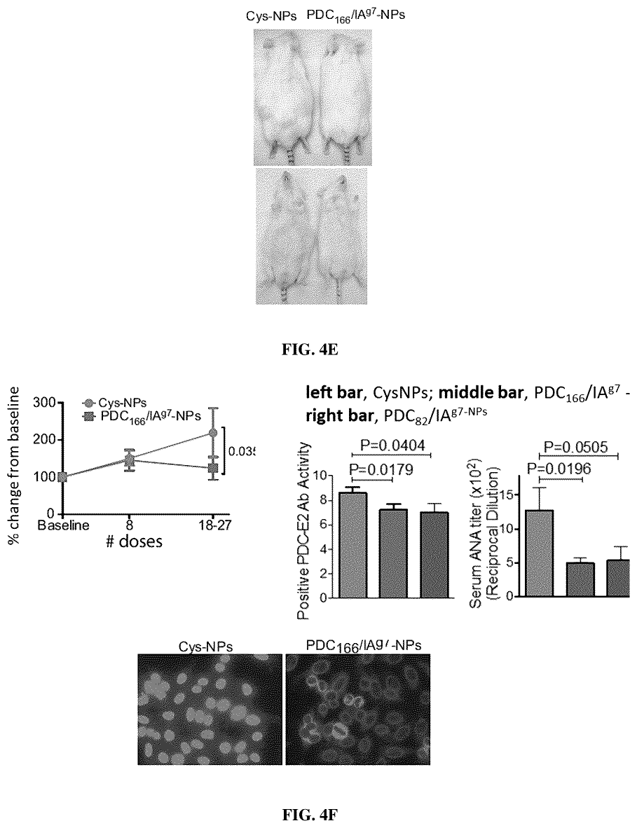

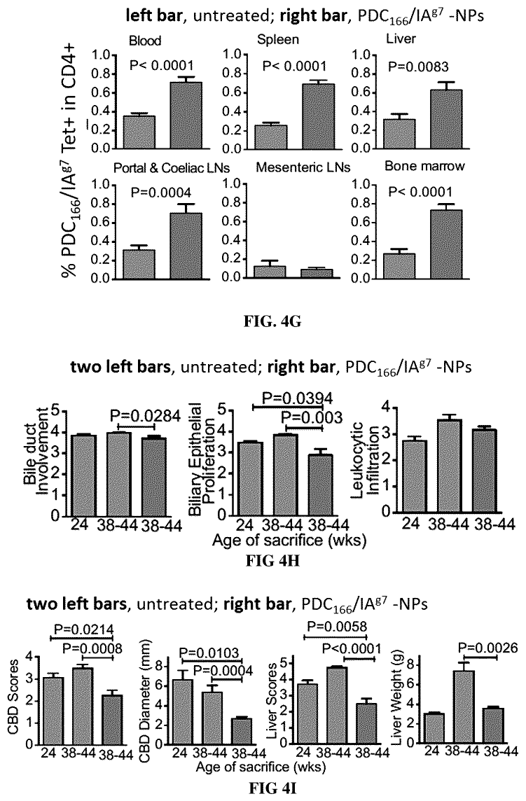

We tested the therapeutic properties of PDC-E2₁₆₆₋₁₈₁/IA^{g7}-, PDC-E2₈₂₋₉₆/I-A^{g7}- and BDC2.5/I-A^{g7}-NPs in 15 week-old NOD.c3c4 mice, an age when liver autoimmunity in these mice is well established. Mice received biweekly doses of 20 μg of pMHC-NPs or an equivalent dose of control (Cys-conjugated NPs; Cys-NPs) for 9-13.5 weeks. Treatment with PDC-E2₁₆₆₋₁₈₁/IA^{g7}-NPs resulted in significant reductions in serum ALT and TB levels (FIG. 4A), bile duct involvement, bile duct epithelial proliferation and leukocyte infiltration (FIG. 4B), common bile duct diameter and macroscopic score (FIG. 4C), liver weight and macroscopic liver scores (FIG. 4D) and abdominal girth (FIG. 4E). Although treatment did not decrease the autoantibody titers found at the initiation of therapy, it clearly blunted the progression of autoantibody formation, as documented by significant reductions in anti-PDC-E2 and anti-nuclear autoantibody titers in mice treated with PDC-E2₁₆₆₋₁₈₁/IA^{g7}-NPs, PDC-E2₈₂₋₉₆/I-A^{g7}-NPs vs. control NPs (FIG. 4F). Additional studies with the second PBC-relevant pMHC-NP compound (PDC-E2₈₂₋₉₆/I-A^{g7}-NPs), and the TID-relevant (but PBC-irrelevant) counterpart (BDC2.5/I-A^{g7}-NPs) confirmed the disease specificity of these compounds (FIGS. 4C and 4D).

Similar results were obtained when treatment was initiated at the peak of disease severity (24 weeks of age). Analyses of mice after 14-20 weeks of therapy (38-44 weeks of age) with PDC-E2₁₆₆₋₁₈₁/IA^{g7}-NPs revealed systemic expansions of TR1-like CD4+ T-cells (FIG. 4G and FIG. 2C) and indicated that the magnitude of the signs of disease (FIGS. 4H and 4I) were significantly lower than those seen at the age when therapy was initiated, suggesting that resolution of liver inflammation by PBC-specific nanomedicines promotes repair of pre-existing liver damage.

### Example 3—Continued Versus Intermittent Treatment

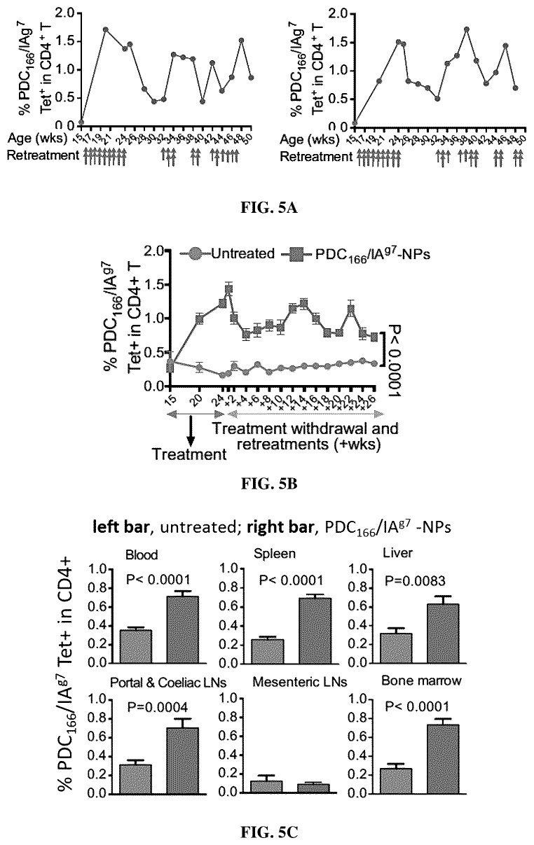

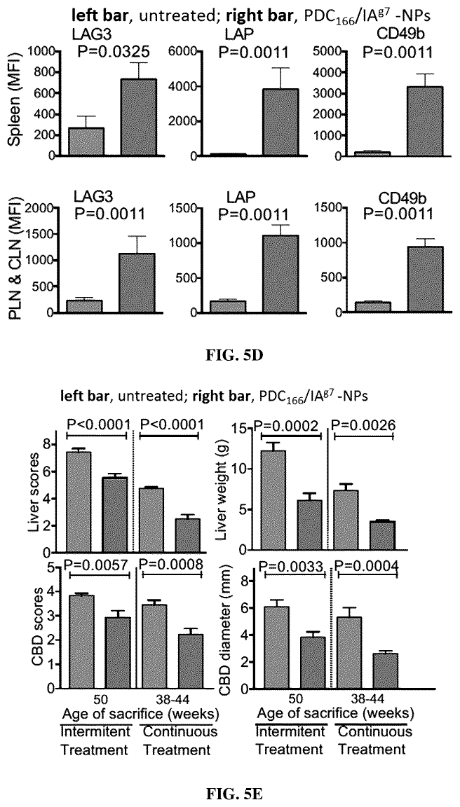

pMHC-NP therapy triggers the formation and expansion of cognate TR1 cells systemically, leading to accumulation of these cells in most lymphoid organs as well as at the site of autoimmune inflammation (See FIGS. 1-3). In addition, these TR1 cells circulate through the bloodstream and their presence in blood can be used as a biomarker to gauge the need for re-treatment. To investigate this, and to ascertain whether circulating levels of cognate TR1-like CD4+ T-cells can in fact be used to guide therapeutic decisions, we withdrew treatment in NOD.c3c4 mice that had been treated with PDC-E2₁₆₆₋₁₈₁/IA^{g7}-NPs from 15 week to 24 weeks of age. We then measured the percentage of PDC-E2₁₆₆₋₁₈₁/IA^{g7} tetramer+CD4+ T-cells in peripheral blood every two weeks, and re-treated animals in which the percentage of tetramer+ cells had declined to about 50% of the value at treatment withdrawal; treatment was again withdrawn when the tetramer+values at the next scheduled measurement had recovered, and repeated this cycle until the mice reached 50 week of age. Although there was considerable variability from mouse to mouse (FIG. 5A), in most animals the peripheral blood tetramer+ TR1 cell content progressively declined to about 50% of the original values within 4-6 weeks after treatment withdrawal but re-treatment of these animals rapidly restored these values (FIG. 5B). Intermittent treatment did not compromise the pharmacodynamic (systemic expansion of TR1-like CD4+ T-cells) (FIGS. 5C and 5D) or the therapeutic effects of treatment, including reduced common bile duct diameter/macroscopic scores and liver weight/macroscopic score (FIG. 5E), as compared to values in untreated mice or mice which had been treated continuously from 24 to 38-44 weeks of age.

### Example 4—pMHC-NPs Versus the Standard of Care

Ursodeoxycholic acid (UDCA, a hydrophilic bile acid) is the standard of care for PBC. See Charatcharoenwitthaya, P. et al. Long-term survival and impact of ursodeoxycholic acid treatment for recurrent primary biliary cirrhosis after liver transplantation. Liver Transpl. 13, 1236-1245. UDCA possesses anti-cholestatic effects and stimulates hepatobiliary secretion, thus protecting cholangiocytes against the toxic effects of hydrophobic bile acids. Although effective in ~50% of patients when given early on in the disease process, it is ineffective at advanced stages of PBC.

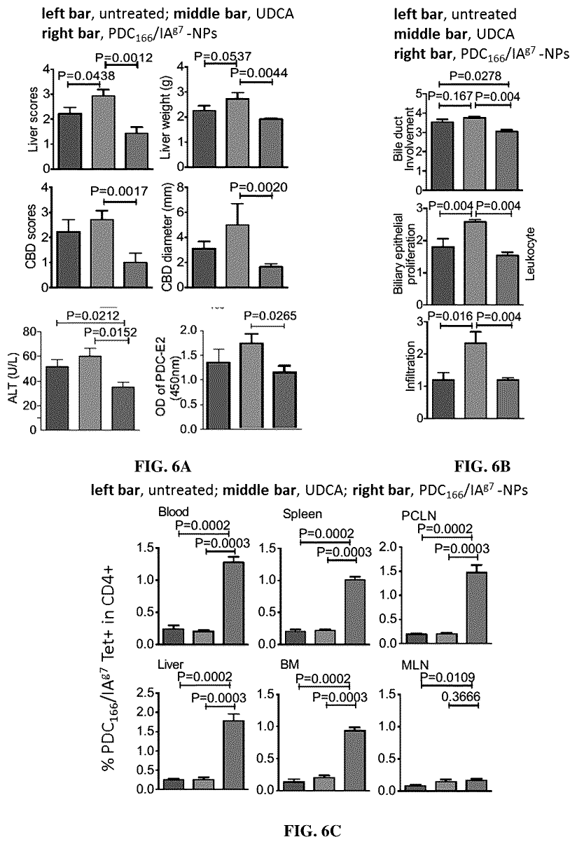

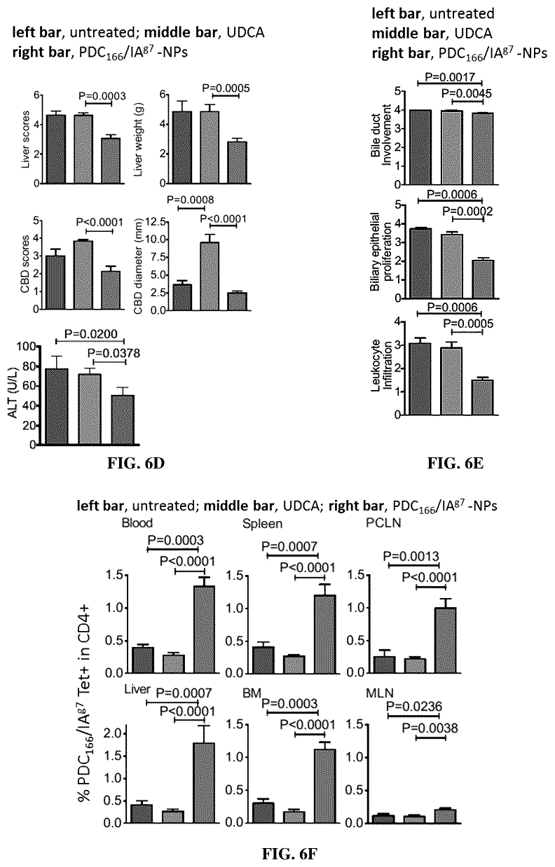

Oral administration of UDCA to 6 weeks-old NOD.c3c4 mice for 9 consecutive weeks via UDCA-supplemented chow had a therapeutic effect on the progression of PBC, as manifested by reductions in liver scores and liver weight, albeit not ALT, CBD scores or CBD diameter (FIG. 6A) and reductions in bile duct involvement and bile duct proliferation, albeit not leukocyte infiltration (FIG. 6B) as compared to untreated mice. However, when UDCA was given at advanced stages of disease progression (24 weeks of age), it had none of these therapeutic effects, except for a very significant reduction in CBD diameter as compared to untreated animals, possibly because of its anti-cholestatic effects (FIGS. 6D and 6E).

In contrast, PDC-E2₁₆₆₋₁₈₁/IA^{g7}-NP treatment had highly significant therapeutic effects in both 6 week-old and 24 week-old animals, as documented by significant reductions in the severity of all read-outs examined (FIGS. 6A-6E). As expected, this was associated with systemic expansion of cognate TR1-like CD4+ T-cells (FIG. 6F).

### Example 5-Disease Suppression Requires IL-10, TGFb and CD4+ T Cells

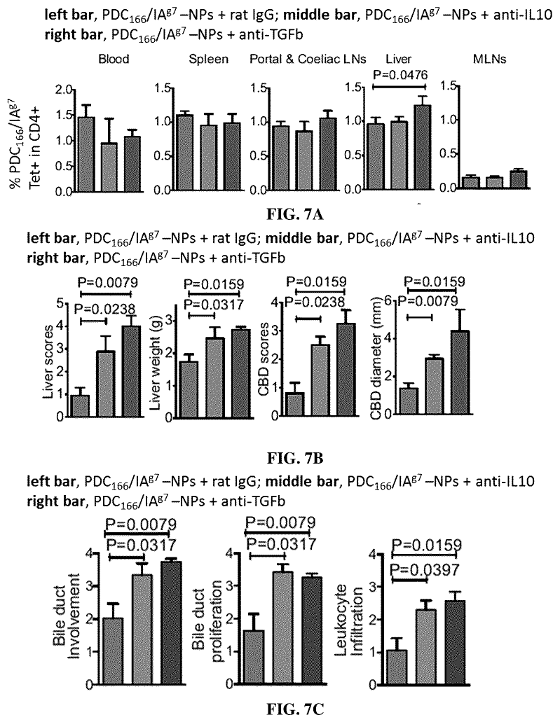

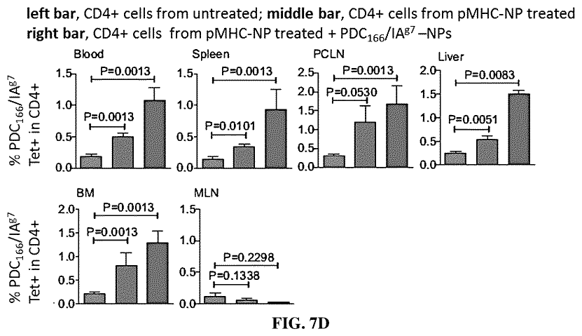

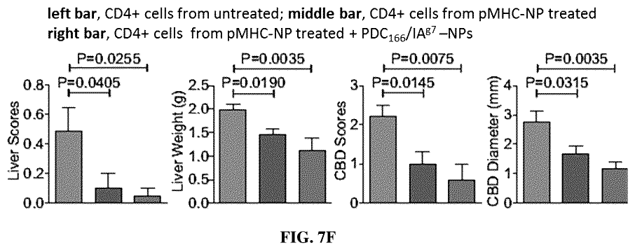

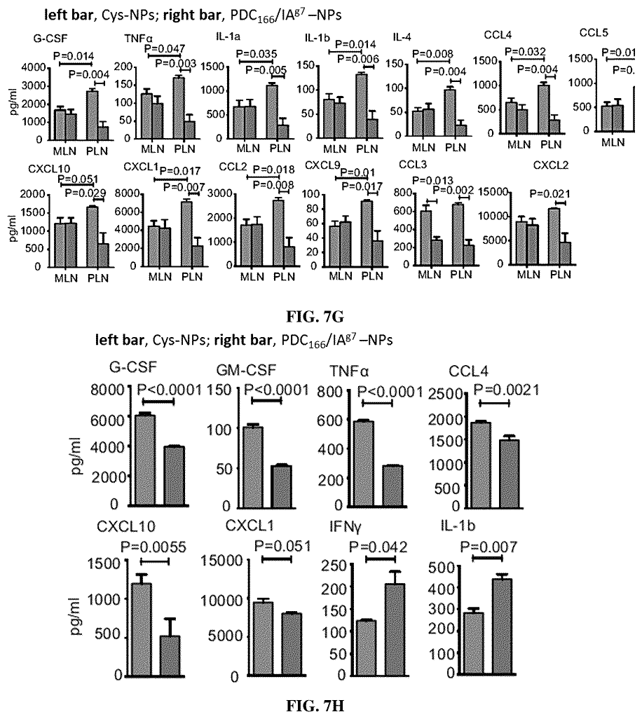

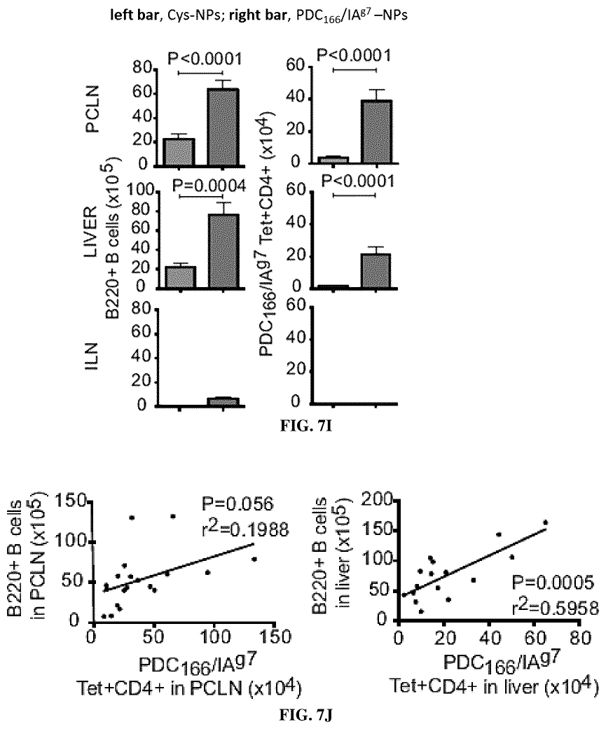

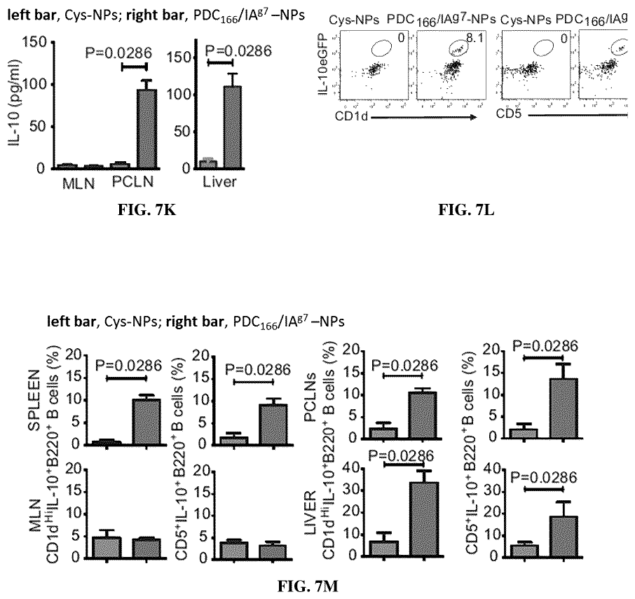

To ascertain if disease reversal by the TR1-like pMHC-NP-expanded PDC-E2₁₆₆₋₁₈₁/IA^{g7} CD4+ T-cells was mediated by the TRI cytokines IL-10 and/or TGFb, we compared the immunological and therapeutic effects of PDC-E2₁₆₆₋₁₈₁/IA^{g7}-NPs on 15 week-old NOD.c3c4 mice treated with blocking anti-IL10 or anti-TGFb mAbs or rat IgG for 5 weeks. Whereas cytokine blockade did not significantly inhibit the expansion of PDC-E2₁₆₆₋₁₈₁/IA 87-specific TR1-like CD4+ T-cells (FIG. 7A), it suppressed their therapeutic effects, as compared to age-matched NOD.c3c4 mice treated with rat-IgG (FIGS. 7B and 7C). Purified splenic CD4+ T-cells from PDC-E2₁₆₆₋₁₈₁/IA^{g7}-NP-treated NOD.c3c4 mice could transfer disease suppression into NOD.scid.c3c4 hosts reconstituted with splenocytes from sick NOD.c3c4 mice, and treatment of the hosts with PDC-E2₁₆₆₋₁₈₁/IA^{g7}-NPs enhanced this effect as shown in FIGS. 7D-7F.

### Example 6—Therapy-Induced Suppression of the Pro-Inflammatory Properties of Local and Proximal APCs

To ascertain whether reversal of PBC by PDC-E2₁₆₆₋₁₈₁/IA^{g7}-NPs was associated with specific suppression of disease-fueling APCs, we compared the cytokine and chemokine profiles of portal (draining) vs. mesenteric (non-draining) lymph node CD11b+ cells and liver Kupffer cells isolated from PDC-E2₁₆₆₋₁₈₁/IA^{g7}-NP and control NP-treated animals. LPS-challenged CD11b+ cells from the portal lymph nodes of control NP-treated animals secreted significantly higher levels of a broad range of pro-inflammatory cytokines and chemokines than their mesenteric lymph node counterparts (FIG. 7G). Conversely, the portal lymph node CD11b+ cells of PDC-E2₁₆₆₋₁₈₁/IA^{g7}-NP-treated mice secreted significantly lower levels of such pro-inflammatory mediators than both their mesenteric lymph node counterparts and the CD11b+ cells isolated from control-NP-treated animals (FIG. 7G). Likewise, the Kupffer cells isolated from the livers of PDC-E2₁₆₆₋₁₈₁/IA^{g7}-NP-treated mice secreted significantly lower levels of some of these mediators (FIG. 7H). Thus, systemic expansion of PDC-E2-specific TR1 CD4+ cells in NOD.c3c4 mice by treatment with PBC-relevant pMHC class II-NPs is associated with dramatic inhibition of the pro-inflammatory properties of local and proximal APC types, likely owing to increased uptake of liver-derived PDC-E2 autoantigenic material.

### Example 7—PBC-Relevant Nanomedicines Promote Local Formation of Regulatory B Cells

Pancreatic beta cell-specific TR1 CD4+ T-cells promote the recruitment of B-cells to the pancreas and its draining lymph nodes, as well as the local formation of anti-diabetogenic IL-10-producing Breg cells. To ascertain if this was also the case for PDC-E2-specific TR1 CD4+ T-cells in the context of PBC, we investigated if there were statistically significant correlations between the absolute numbers of B-cells and PDC-E2₁₆₆₋₁₈₁/IA^{g7}-specific TR1 cells in the liver, portal and mesenteric lymph nodes of PDC-E2₁₆₆₋₁₈₁/IA^{g7}-NP-treated mice. The livers and the portal, but not the mesenteric lymph nodes of PDC-E2₁₆₆₋₁₈₁/IA^{g7}-NP-treated mice harbored significantly higher numbers of PDC-E2₁₆₆₋₁₈₁/IA^{g7}-tetramer+ cells and B-cells than those from control-NP-treated animals (FIG. 7I). Furthermore, the tetramer+ and B-cell numbers in both liver and portal lymph nodes were statistically correlated; no such correlation was seen in the mesenteric lymph nodes of PDC-E2₁₆₆₋₁₈₁/IA^{g7}-NP-treated mice (FIG. 7J). Thus, enhanced recruitment of cognate TR1 cells to the liver and liver-draining lymph nodes of PDC-E2₁₆₆₋₁₈₁/IA^{g7}-NP-treated mice promotes the local accumulation of B-cells.

To ascertain whether the liver and portal lymph node B-cells of PDC-E2₁₆₆₋₁₈₁/IA^{g7}-NP-treated mice might be enriched for Breg cells, we compared the ability of the corresponding B-cells to produce IL-10 in response to LPS stimulation. The liver and portal, but not the mesenteric lymph node B-cells of PDC-E2₁₆₆₋₁₈₁/IA^{g7}-NP-treated mice produced significant levels of IL-10; neither the liver nor the portal lymph node B-cells of control NP-treated animals produced IL-10 (FIG. 7K). Thus, TR1 CD4+ T-cell-enhanced recruitment of B-cells to liver and draining lymph nodes is associated with local formation of IL-10-producing B-cells.

To further substantiate a direct relationship between TR1 cell recruitment and Breg cell formation, we ascertained the ability of PDC-E2₁₆₆₋₁₈₁/IA^{g7}-specific TR1 cells that accumulate in the spleen, liver and portal (but not mesenteric) lymph nodes of PDC-E2₁₆₆₋₁₈₁/IA^{g7}-treated mice to promote the differentiation of PDC-E2₁₆₆₋₁₈₁ peptide-pulsed conventional (IL-10/eGFP−) B-cells from NOD.Il10-eGFP reporter mice into CD1dhigh/CD5+/eGFP+progeny. As shown in FIGS. 7K-7M, there was a clear formation of Breg cells in spleen, liver and portal lymph nodes (containing cognate TR1 cells) but not in the mesenteric lymph nodes (lacking cognate TR1 cells). When taken together, these results indicate that PDC-E2-specific TR1 cells promote the recruitment and differentiation of conventional B-cells into Breg-like cells.

### Example 8—Systemic Immunity is Spared

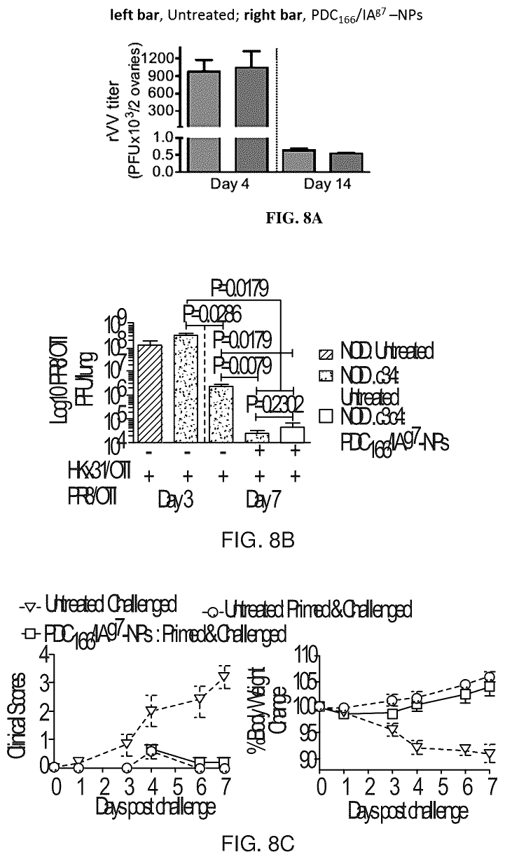

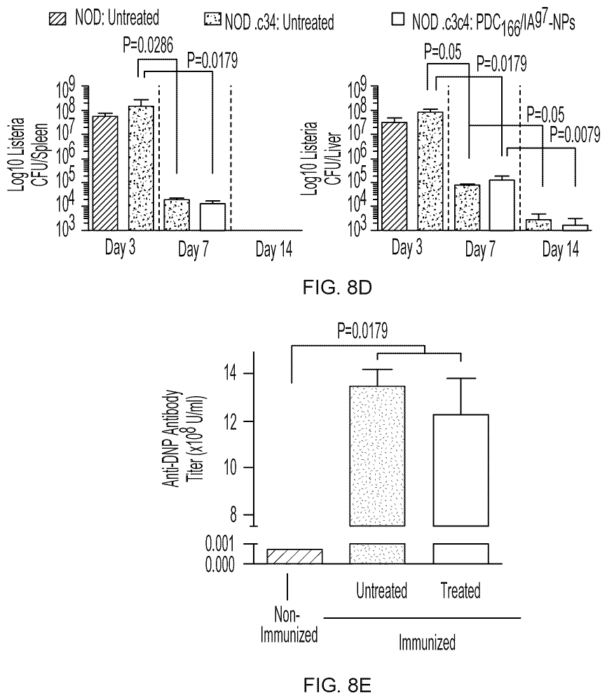

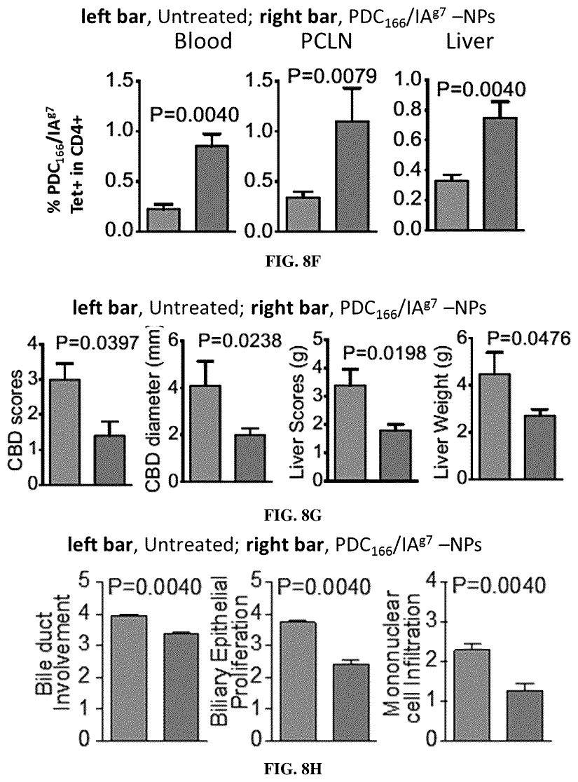

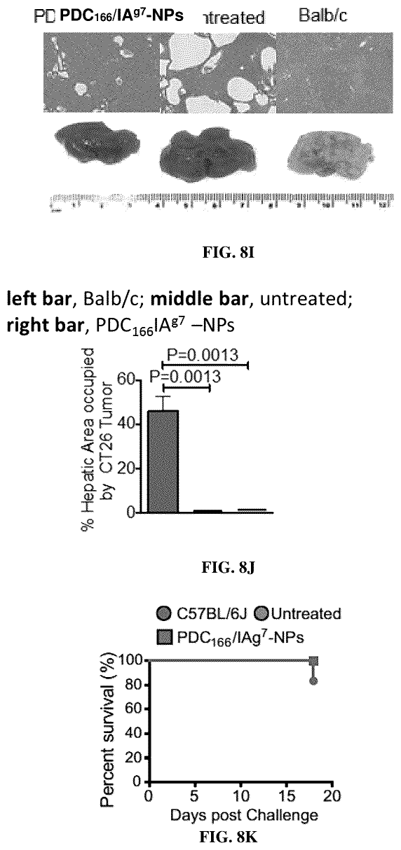

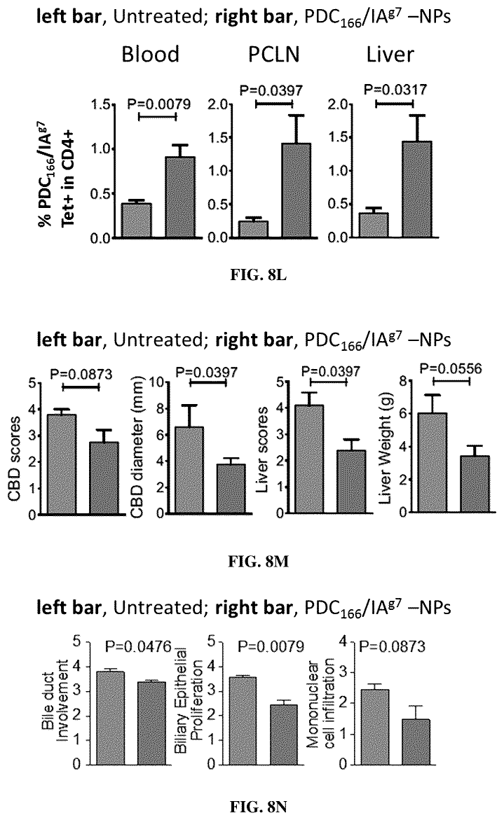

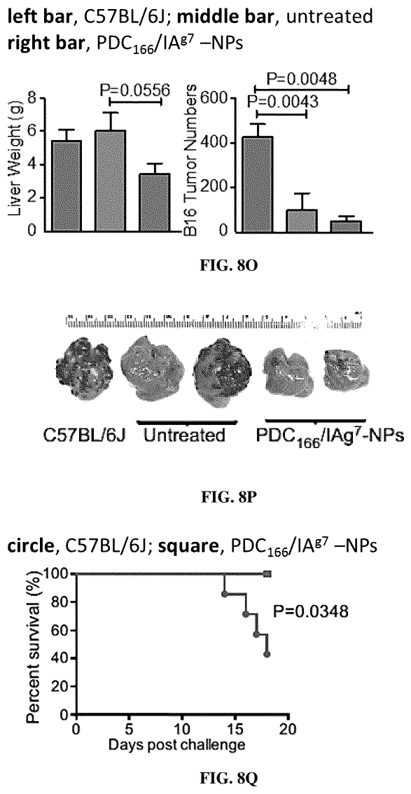

To ascertain if persistent expansion of PDC-E2₁₆₆₋₁₈₁/IA^{g7}-specific TR1 cells results in suppression of general immunity, we compared the ability of PDC-E2₁₆₆₋₁₈₁/IA^{g7}-NP-treated NOD.c3c4 mice to clear a systemic vaccinia virus infection, to mount effector T-cell responses against a lethal influenza infection, to mount protective immunity against an intracellular bacterial infection (Listeria), and to mount local immune responses against allogeneic tumor liver metastases. Cohorts of NOD.c3c4 mice received biweekly doses of PDC-E2₁₆₆₋₁₈₁/IA^{g7}-NPs or control NPs for 9 weeks. At the end of therapy the mice were given an intravenous injection of recombinant vaccinia virus. The viral titers in the ovaries of both cohorts of females 14 days after infection were similar in both cohorts of mice and substantially lower than those found at the peak of infection, indicating that pMHC-NP therapy did not impair cellular immunity against the virus-infected cells (FIG. 8A).

To probe this further, we infected PDC-E2₁₆₆₋₁₈₁/IA^{g7}-NP-treated or untreated NOD.c3c4 mice with a laboratory strain of influenza HKx31 (H3N2) i.p. to induce heterologous immunity against a subsequent, potentially lethal infection with an H1N1 strain of Influenza (PR8) given via the intranasal route, that shares MHC class I-restricted epitopes. As shown in FIGS. 8B and 8C, treated and untreated mice mounted protective immunity against the PR8 infection, as documented by viral load in lung tissue and clinical signs of active infection.

Similar results were obtained in mice infected with the intracellular pathogen Listeria monocytogenes (LM), which does not normally cause chronic infections. LM-infected PDC-E2₁₆₆₋₁₈₁/IA^{g7}-NP-treated and untreated NOD.c3c4 mice were equally efficient at clearing the bacteria from both the spleen and liver, consistent with unimpaired immunity against this intracellular pathogen (FIG. 8D).

PDC-E2₁₆₆₋₁₈₁/IA^{g7}-NP-treated and untreated NOD.c3c4 mice also produced similar titers of anti-dinitrophenyl (DNP) antibodies upon immunization with the hapten-carrier conjugate DNP-keyhole limpet haemocyanin (KLH) (FIG. 8E).

Lastly, systemic expansion and liver accumulation of PBC-suppressing PDC-E2-specific TR1-like CD4+ T-cells (FIGS. 8F and 8L) did not impair the ability of treated NOD.c3c4 mice to mount immune responses against allogeneic colon carcinoma (CT26) and melanoma (B16) liver metastases arising upon intra-splenic injection, as compared to untreated NOD.c3c4 mice or syngeneic hosts (Balb/c and C57BL/6J, respectively) (FIGS. 8F-8K and 8L-8Q). Thus, despite targeting a ubiquitously expressed antigen, PDC-E2₁₆₆₋₁₈₁/IA^{g7}-specific TR1 cells do not impair immunity against foreign or tumor antigens.

### Example 9—Humanized Mice with PBC

DRB4*0101 and DRB1*0801 have been associated with PBC in some studies. To ascertain the HLA haplotypic diversity in PBC, we did high-resolution HLA-DRB1-typing of 154 patients with PBC from Spain. 40.3% of patients expressed DRB1*0701, 25% were DRB1*0301+ and 14% were DRB1*0801+. Since DRB1*0701+ and haplotypes carrying other DRB1 alleles carry the oligomorphic HLA-DRB4 locus, we also typed these patients for DRB4*0101. 61.7% of all PBC patients carried the DRB4*0101 allele.

Several T-cell epitopes from PDC-E2 binding to two of these HLA-DRB types (DRB4*0101 and DRB1*0801) have been described, including PDC-E2₂₄₉₋₂₆₂ (GDLLAEIETDKATI; DRB4*0101-binder), PDC-E2₁₂₂₋₁₃₅ (GDLIAEVETDKATV; also DRB4*0101-binder), PDC-E2₂₄₉₋₂₆₃ (GDLLAEIETDKATIG; DRB1*0801) and PDC-E2₆₂₉₋₆₄₃ (AQWLAEFRKYLEKPI; DRB1*0801). We therefore expressed and purified PDC-E2₁₂₂₋₁₃₅/DRB4*0101, PDC-E2₂₄₉₋₂₆₂/DRB4*0101, and PDC-E2₆₂₉₋₆₄₃/DRB1*0801 complexes and produced iron oxide nanoparticles displaying each of these complexes, as described.

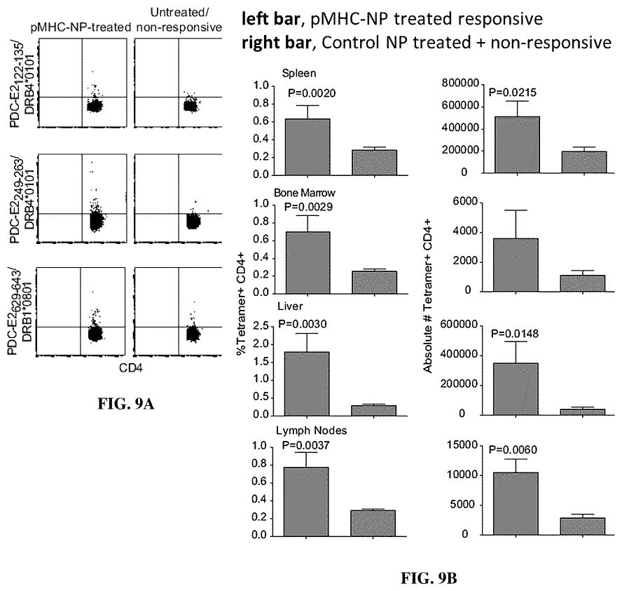

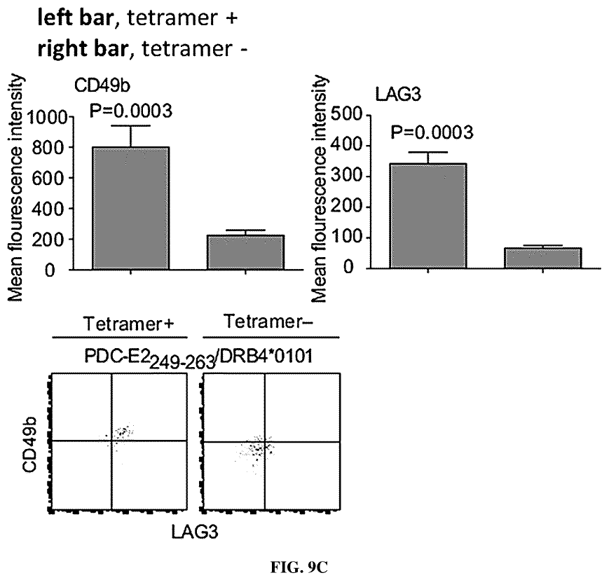

To investigate the translational significance of the above observations, we tested the ability of these three human PBC-relevant pMHC class II-NPs to expand cognate TR1-like CD4+ T-cells in NOD.scid/Il2rg−/− (NSG) hosts reconstituted with PBMCs from 11 DRB4*0101+ and 5 DRB1*0801+PBC patients (PBL-NSG mice, Tables 2, 3 and 4). PBMC-transfused NSG hosts were then treated with 8-10 doses of 20 μg pMHC-NPs intravenous (twice/week for 5 weeks). One mouse did not engraft and three others died from GvHD prior to termination of treatment. As controls, we transfused a second mouse per donor and treated it with control (non-pMHC-coated NPs). Expansions of cognate CD4+ T-cells were analyzed in the spleens, liver, portal/celiac and axillary lymph nodes. We saw expansion of tetramer+CD49b+LAG-3+CD4+ T-cells in the spleen and/or liver and LNs from all 6/6 PBL-NSG mice treated with PDC-E2₁₂₂₋₁₃₅/DRB4*0101-NPs, 5/6 PBL-NSG mice treated with PDC-E2₂₄₉₋₂₆₂/DRB4*0101-NPs and 4/5 PBL-NSG mice treated with PDC-E2₆₂₉₋₆₄₃/DRB1*0801-NPs vs. the untreated controls (Tables 2, 3, and 4). Treated responsive mice had significantly higher percentages and absolute numbers of tetramer+ cells in spleen, liver and lymph nodes (FIGS. 9A and 9B) than control NP-treated or unresponsive mice, and these cells expressed the TR1 markers CD49b and LAG-3 (FIG. 9C).

TABLE 2
Table 2 hPDC-E2(122-135)-DRB4-PFM-010616 (30 mcg/dose).

| | | | | Spleen | | Liver | | LN | | |
|---|---|---|---|---|---|---|---|---|---|---|
| | | | Anti- | % hCD4 | % hCD4 | % hCD4 | % hCD4 | % hCD4 | % hCD4 | |
| | | Age | mitochondrial | Treated | Untreated | Treated | Untreated | Treated | Untreated | |
| ID | Gender | (yr) | Abs | # of cells | # of cells | # of cells | # of cells | # of cells | # of cells | Outcome |
| B013 | F | 79 | + | 0.055 | 0.303 | 2.288 | 0.302 | 0.863 | 0.274 | + |
| | | | | | | | | (A) | | |
| | | | | 63756 | 141561 | 300444 | 39069 | 23760 | 154 | |
| B009 | F | 76 | + | 1.253 | 0.291 | 0.199 | 0.307 | * | | + |
| | | | | | (249-NP) | | (249-NP) | | | |
| | | | | 732729 | 308949 | 7047 | 11853 | * | | |
| B005 | M | 74 | + | No engraftment | | | | | | |
| B001 | M | 59 | + | 0.319 | 0.296 | 1.625 | 0.296 | 1.082 | 0.312 | + |
| | | | | | | | | (A) | | |
| | | | | 186539 | 291863 | 256354 | 62871 | 28524 | 1874 | |
| B021 | F | 68 | + | 0.767 | 0.309 | 0.343 | 0.296 | 0.728 | 0.3 | + |
| | | | | | | | | (A) | | |
| | | | | 849816 | 509331 | 18691 | 28993 | 15072 | 5721 | |
| B011 | F | 71 | + | 0.169 | 0.304 | 0.091 | 0.294 | 0.425 | 0.291 | + |
| | | | | | | | | (L) | | |
| | | | | 202383 | 209370 | 6658 | 151772 | 120 | 755 | |
| B018 | F | 56 | + | Dead | | | | | | |
| B012 | F | 71 | + | 0.709 | 0.298 | | | 0.722 | 0.290 | + |
| | | | | | | | | (A) | (A) | |

| | | | | |
|---|---|---|---|---|
| 341920 | 124050 | ND | 5602 | 2101 |

DRB4*01:01 patients

TABLE 3
Table 3 hPDC-E2(249-262)-DRB4-PFM-010616 (40 mcg/dose).

| | | | | Spleen | | Liver | | LN | | |
|---|---|---|---|---|---|---|---|---|---|---|
| | | | Anti- | % hCD4 | % hCD4 | % hCD4 | % hCD4 | % hCD4 | % hCD4 | |
| | | Age | mitochondrial | Treated | Untreated | Treated | Untreated | Treated | Untreated | |
| ID | Gender | (yr) | Abs | # of cells | # of cells | # of cells | # of cells | # of cells | # of cells | Outcome |
| B013 | F | 79 | + | 0.078 | 0.31 | 4.925 | 0.295 | 0.621 | 0.245 | + |
| | | | | | | | | (L) | | |
| | | | | 105174 | 164589 | 1124257 | 35712 | 33121 | 275 | |
| B009 | F | 76 | + | 0.028 | 0.307 | 0.515 | 0.297 | * | | + |
| | | | | | (122-NP) | | (122-NP) | | | |
| | | | | 31807 | 192099 | 20215 | 10866 | * | | |
| B004 | F | 63 | + | 0.427 | 0.294 | 0.094 | 0.315 | 0.089 | 0.293 | + |
| | | | | | | | | (A) | | |
| | | | | 268565 | 228575 | 2990 | 299574 | 526 | 11251 | |
| B012 | F | 70 | + | 0.264 | 0.297 | 0.078 | 0.3 | 0.059 | 0.313 | − |
| | | | | | | | | (L) | | |
| | | | | 41467 | 147568 | 1034 | 24584 | 1513 | 6160 | |
| B001 | M | 59 | + | Dead | | | | | | |
| B021 | F | 68 | + | 1.612 | 0.31 | 1.943 | 0.289 | 2.589 | 0.32 | + |
| | | | | | | | | (L) | | |
| | | | | 1368420 | 514802 | 1174100 | 35690 | 9491 | 788 | |
| B019 | F | 73 | + | Dead | | | | | | |
| B014 | F | 72 | + | 0.709 | 0.295 | 0.588 | 0.305 | 1.096 | 0.299 | + |
| | | | | | | | | (A) | | |
| | | | | 604820 | 162521 | 76559 | 30198 | 5557 | 1271 | |
| | | | | 416501 | 181855 | 4960 | 4517 | 3740 | 2232 | |

DRB4*01:01 patients

TABLE 4
Table 4 hPDC-E2(629-643)-DR8-PFM-010616 (30 mcg/dose).

| | | | | Spleen | | Liver | | LN | | |
|---|---|---|---|---|---|---|---|---|---|---|
| | | | Anti- | % hCD4 | % hCD4 | % hCD4 | % hCD4 | % hCD4 | % hCD4 | |
| | | Age | mitochondrial | Treated | Untreated | Treated | Untreated | Treated | Untreated | |
| ID | Gender | (yr) | Abs | # of cells | # of cells | # of cells | # of cells | # of cells | # of cells | Outcome |
| B022 | F | 72 | + | 0.024 | 0.3 | 0.619 | 0.293 | No cells | 0.295 | + |
| | | | | | | | | | (L) | |
| | | | | 662 | 48190 | 26 | 29984 | | 260 | |
| B021 | F | 68 | + | 0.725 | 0.317 | 3.185 | 0.3 | 1.103 | 0.301 | + |
| | | | | | | | | (A) | | |
| | | | | 1095456 | 562089 | 455716 | 36134 | 22752 | 8080 | |
| B020 | F | 70 | + | 0.303 | 0.308 | 0.18 | 0.297 | No cells | | − |
| | | | | 38207 | 114160 | 19765 | 46701 | | | |
| B025 | F | 69 | + | 0.404 | 0.308 | 2.895 | 0.294 | 0.501 | 0.319 | + |
| | | | | | | | | (A) | (A) | |
| | | | | 59552 | 49721 | 78551 | 8747 | 740 | 466 | |
| B023 | F | 75 | + | 0.745 | 0.292 | 0.283 | 0.292 | 0.371 | 0.297 | + |
| | | | | | | | | (A) | | |

DRB1*08:01 patients

### Example 10—Disease Versus Organ Specificity

Given the large autoantigenic load of an organ such as the liver as compared to smaller organs, like the endocrine pancreas, and the fact that PDC-E2 is an autoantigen expressed in virtually all cell types, our results begged the question of whether PBC-relevant nanomedicines (i.e. PDC-E2₁₆₆₋₁₈₁/IA^{g7}-NP) are disease (PBC)- or organ (liver)-specific, or also able to blunt liver-distal autoimmune inflammation.

Primary sclerosing cholangitis (PSC) is a chronic cholestatic disease characterized by inflammation of intra- and extra-hepatic bile ducts leading to a fibro-obliterative cholangitis with periductal fibrosis around medium and large bile ducts and degenerative changes of the biliary epithelium, which progresses to portal and biliary cirrhosis and finally liver cirrhosis. Human PSC is frequently associated with inflammatory bowel disease and accompanied by a high prevalence of atypical perinuclear anti-neutrophil cytoplasmic (pANCA) but not anti-mitochondrial autoantibodies. See Fickert, P. et al. Characterization of animal models for primary sclerosing cholangitis (PSC). J. Hepatol. 60, 1290-1303. Abcb4 gene knockout mice spontaneously develop a form of sclerosing cholangitis that is remarkably similar to human PSC and is caused by damage of bile duct cells by impaired biliary phospholipid secretion. See Pollheimer, M. J. & Fickert, P. Animal models in primary biliary cirrhosis and primary sclerosing cholangitis. Clin. Rev. Allergy Immunol. 48, 207-217, doi:10.1007/s12016-014-8442-y (2015).

Autoimmune Hepatitis (AIH) is characterized by a portal mononuclear cell infiltration of the liver parenchyma that is associated with presence of anti-nuclear and/or smooth muscle (AIH type 1) or anti-liver kidney microsomal or anti-liver cytosol type 1 autoantibodies, which specifically target the microsomal cytochrome P450IID6 (CYP2D6) or formiminotransferase cyclodeaminase (FTCD), respectively (AIH Type 2). See Longhi, M. S. et al. Aetiopathogenesis of autoimmune hepatitis. J. Autoimmun. 34, 7-14. Recently, it has been shown that infection of NOD mice with replication-defective adenoviruses encoding the human liver autoantigen formiminotransferase cyclodeaminase (Ad-FTCD) triggers a form of chronic autoimmune hepatitis that resembles AIH type 2. See Hardtke-Wolenski, M. et al. Genetic predisposition and environmental danger signals initiate chronic autoimmune hepatitis driven by CD4+ T cells. Hepatology 58, 718-728.

The large bile duct and parenchymal liver damage that underlie PSC and AIH may trigger the release of PDC-E2 and the priming of cognate autoreactive CD4+ T-cells capable of responding to PDC-E2₁₆₆₋₁₈₁/IA^{g7}-NP therapy. If so, therapy should trigger the expansion of TR1-like PDC-E2₁₆₆₋₁₈₁/IA^{g7}-specific CD4+ T-cells and suppression of local inflammation upon recognition of local and proximal PDC-E2-loaded APCs. Alternatively, the amount of PDC-E2 shed into the inflammatory milieu in PSC and/or AIH may be insufficient to generate PDC-E2₁₆₆₋₁₈₁/IA^{g7}-experienced CD4+ T-cells, hence an immunological and therapeutic response to PDC-E2₁₆₆₋₁₈₁/IA^{g7}-NPs.

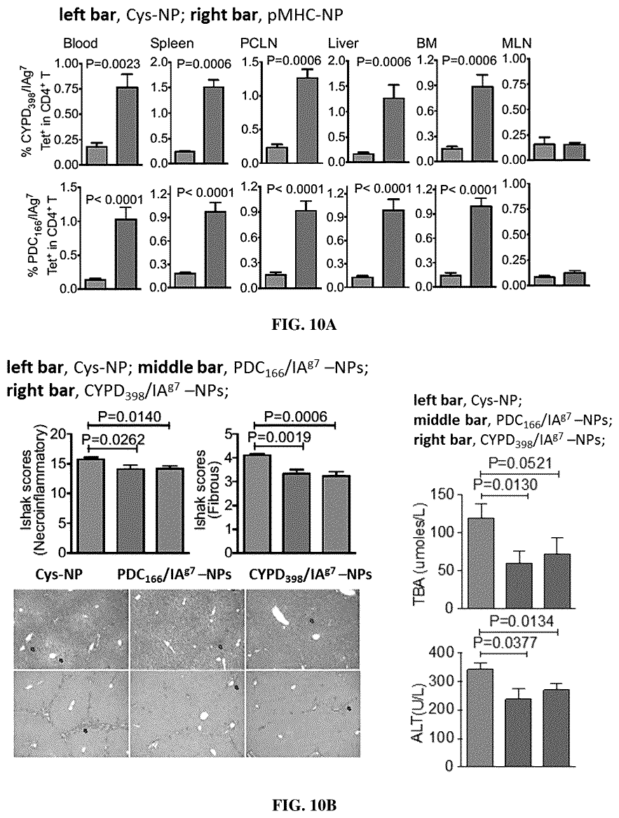

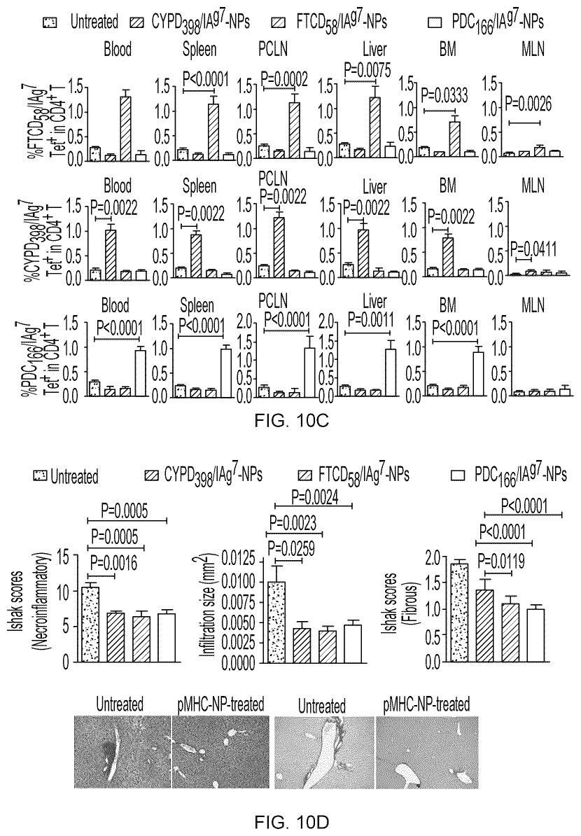

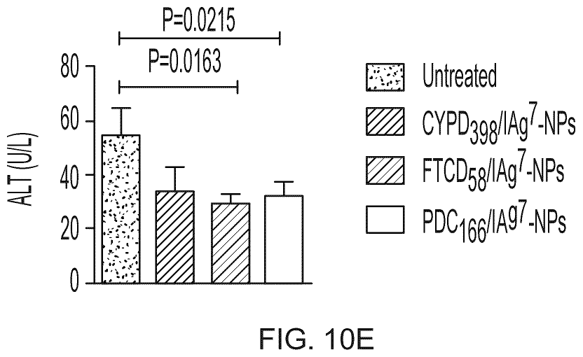

To test these alternative possibilities, we first investigated the ability of PDC-E2₁₆₆₋₁₈₁/IA^{g7}-NPs to trigger the expansion of cognate TR1-like CD4+ T-cells and revert PSC in NOD.Abcb4^{-/-} mice. Remarkably, PDC-E2₁₆₆₋₁₈₁/IA^{g7}-NP triggered the systemic expansion of cognate TR1-like CD4+ T-cells in these animals and reverted established disease, as compared to control NP-treated controls as shown in FIGS. 10A-10B and FIGS. 11A and 11B.

We next investigated whether this was also true in Ad-FTCD-induced AIH. We compared the pharmacodynamic and therapeutic activities of both mFTCD₅₈₋₇₂/IA^{g7}-NPs and CYPD₃₉₈₋₄₁₂/IA^{g7}-NPs (AIH-relevant) with those of PDC-E2₁₆₆₋₁₈₁/IA^{g7}-NPs (PBC-relevant) in NOD mice infected with Ad-FTCD. All three compounds triggered the formation and expansion of cognate TR1-like CD4+ T-cells in these animals to a similar extent (FIG. 10C and FIGS. 12A and 12B) as compared to untreated Ad-FTCD-infected animals, and this was accompanied by significant reductions in liver inflammation as shown in FIGS. 10C and 10D, and serum ALT levels as shown in FIG. 10E.

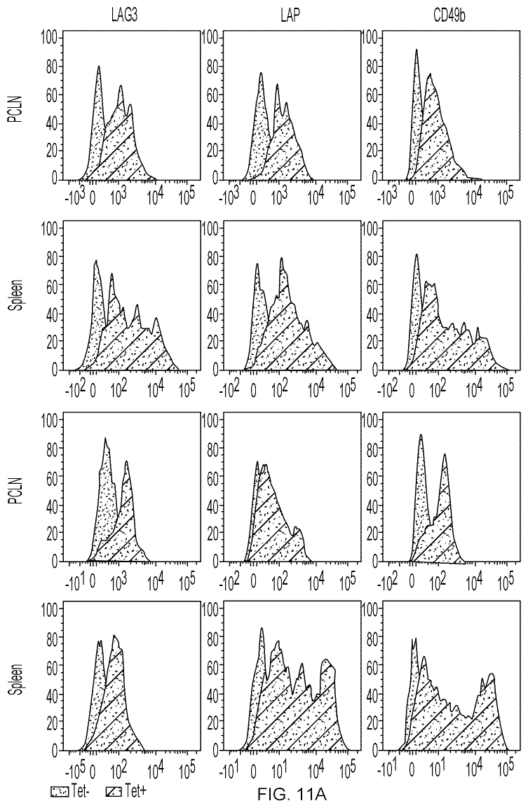

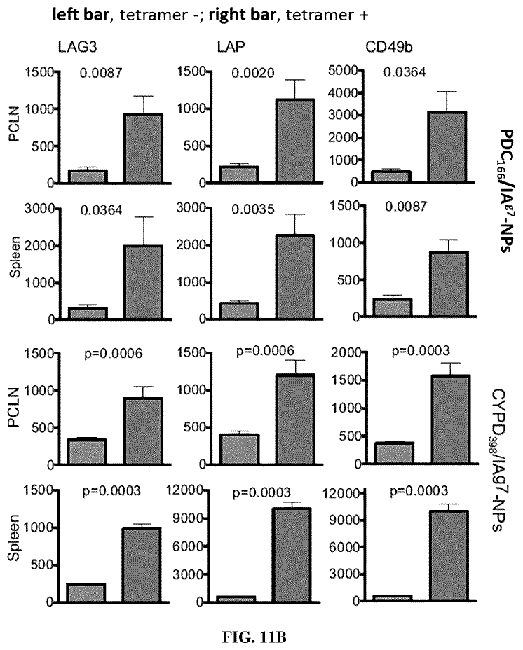

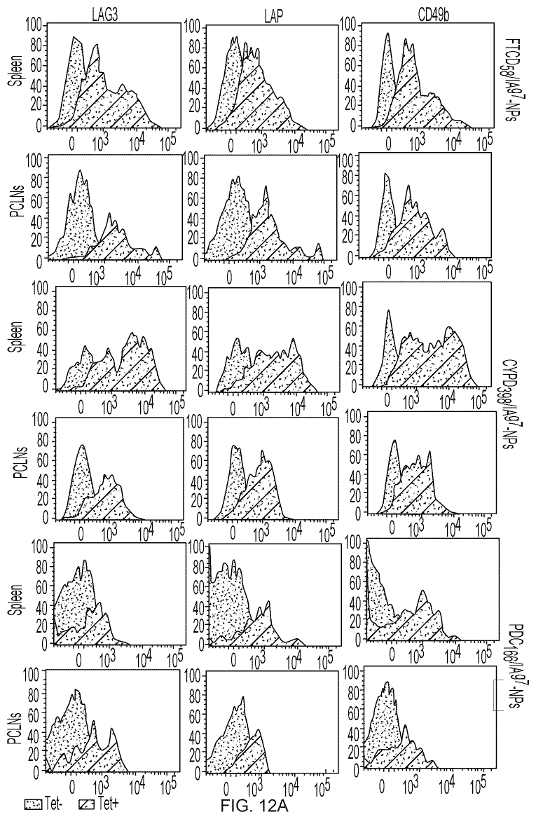

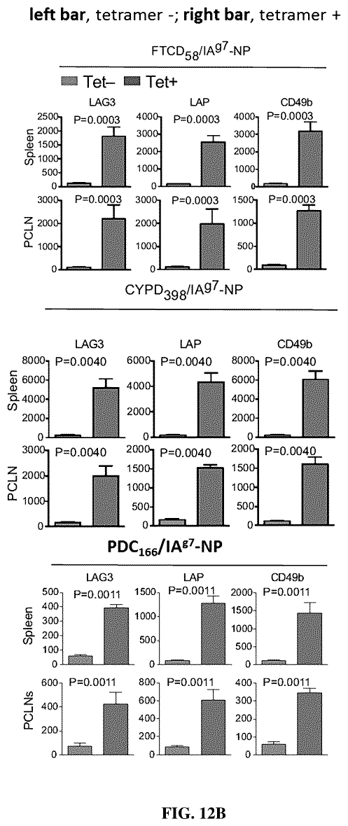

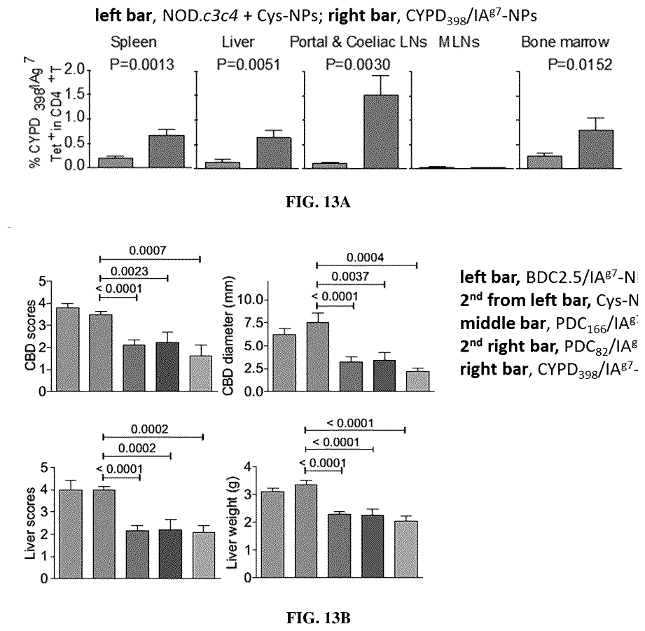

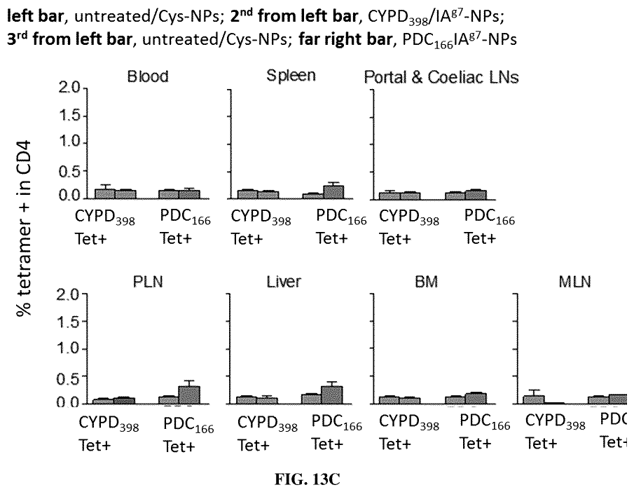

This ability of ubiquitous autoantigen-based pMHC-nanomedicines to blunt liver autoimmunity in an organ rather than disease-specific manner also occurred in NOD.c3c4 mice treated with CYPD₃₉₈₋₄₁₂/IA^{g7}-NPs (FIGS. 13A-13C). In fact, the latter were as efficient as PDC-E2₁₆₆₋₁₈₁/IA^{g7}-NPs at expanding cognate TR1 cells (FIG. 13A), and blunting PBC in 15 week-old mice (FIG. 13B). In contrast, neither PDC-E2₁₆₆₋₁₈₁/IA^{g7}-NPs nor CYPD₃₉₈₋₄₁₂/IA^{g7}-NPs triggered the expansion of cognate TR1 CD4+ T-cells in 10 week-old pre-diabetic NOD mice (FIG. 13C), unlike the case for beta cell-specific BDC2.5/IA^{g7}-, IGRP₄₋₂₂/IA^{g7}- or IGRP₁₂₈₋₁₄₅/IA^{g7}-NPs, presumably because the PDC-E2 and CYPD content in beta cells is insufficient to prime the activation of cognate CD4+ T-cells.

Collectively, these observations suggest that abundant levels of PDC-E2 (mitochondrial), CYPD₂D6 and FTCD antigens (Golgi-resident or cytoplasmic, respectively) are delivered to local and proximal APCs upon hepatocyte (AIH) or bile duct epithelial cell damage (PBC and PSC), enabling autoreactive CD4+ T-cell priming, cognate TR1 cell generation by pMHC-NPs and suppression of local and proximal autoantigen-loaded APCs.

### Example 11—Therapeutic Effects in Another PBC Model

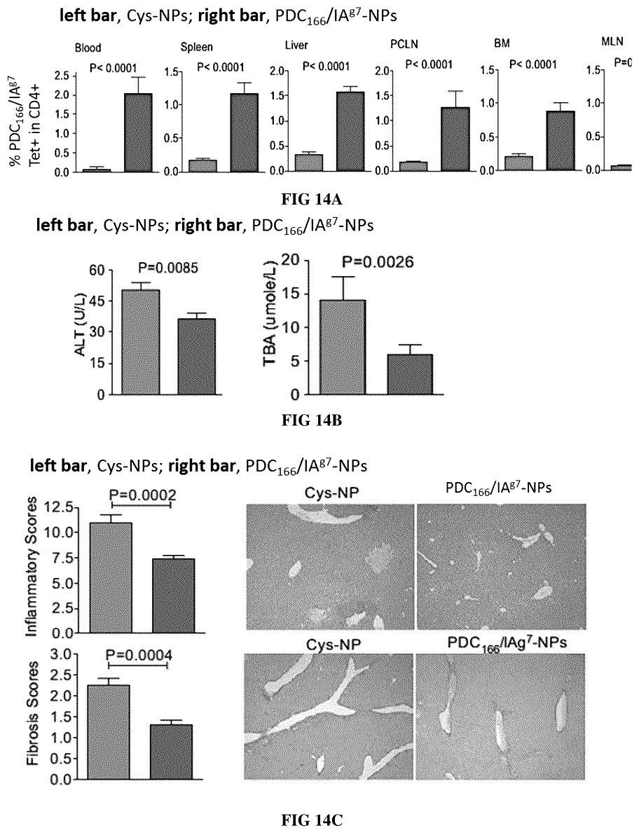

The NOD.c3c4 model does not fully recapitulate the immunopathology of human PBC, characterized by female prevalence, progression to liver fibrosis and absence of liver cyst formation. B6 mice carrying a deletion of the IFNγ 3′-untranslated region adenylate uridylate-rich element (ARE) (ARE-Del+/−) have a dysregulated Ifng locus, and develop a form of PBC that, like the human disease, primarily affects females and is associated with up-regulation of TBA, production of anti-PDC-E2 autoantibodies, portal duct and lobular liver inflammation, bile duct damage, granuloma formation and fibrosis. FIG. 14A-C show that treatment of (NODxB6.ARE-Del−/−) F1 mice with PDC-E2₁₆₆₋₁₈₁/IA^{g7}-NPs suppressed the upregulation of TBA and ALT levels, liver inflammation and fibrosis, as compared to mice treated with control NPs.

### Example 12—Ubiquitous Autoantigen pMHCII-NPs Spare General Immunity Despite Harboring PDC-E2 Autoreactive TRI-Like CD4+ T-Cells

PDC-E2₁₆₆₋₁₈₁/IA^{g7}-NP-treated or untreated NOD.c3c4 mice were infected with a laboratory strain of influenza (HKx31-H3N2-) i.p. to induce heterologous immunity against a lethal infection with an H1N1 strain of Influenza (PR8) given via the intranasal route. As shown in FIGS. 8B-8C, pMHCII-NP treated NOD.c3c4 mice mounted protective immunity against the PR8 infection, as documented by viral load in lung tissue and clinical signs of active infection, despite systemic presence of cognate TR1-like CD4+ T-cells as shown in FIG. 15A.

Similar results were obtained in mice infected with the intracellular pathogen Listeria monocytogenes (LM). LM-infected PDC-E2₁₆₆₋₁₈₁/IA^{g7}-NP-treated and untreated NOD.c3c4 mice were equally efficient at clearing the bacteria from both the spleen and liver as shown in FIG. 8D, consistent with unimpaired immunity against this intracellular pathogen as shown in FIG. 15B.

### Example 13—Ubiquitous Autoantigen pMHC-NPs Treat EAE in Mice

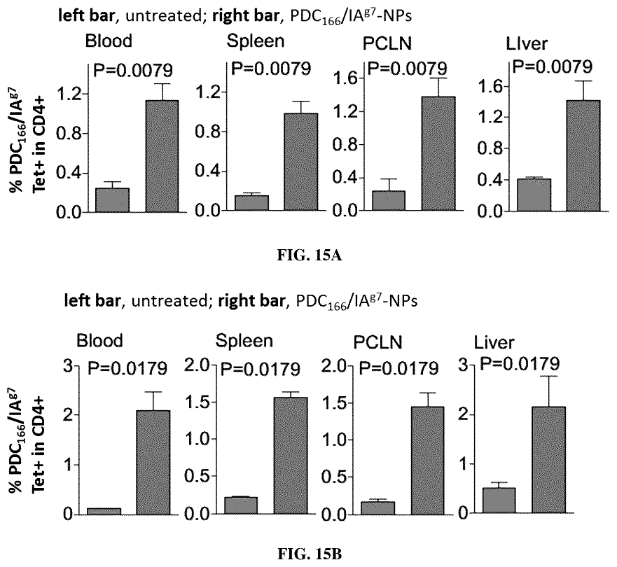

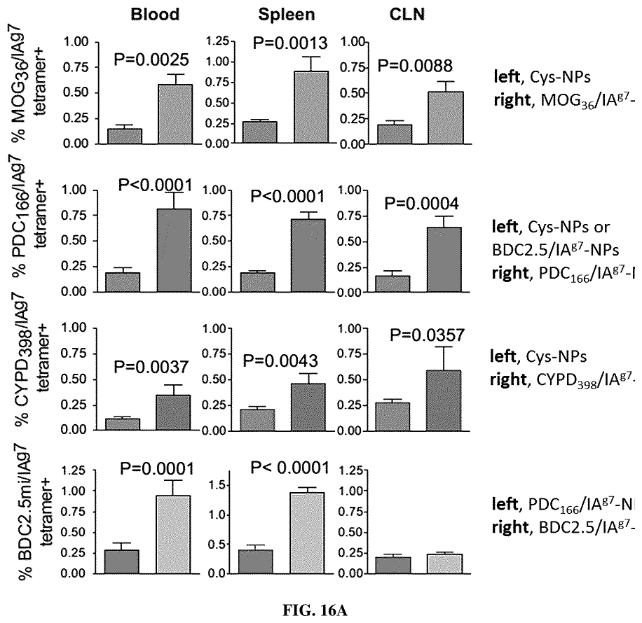

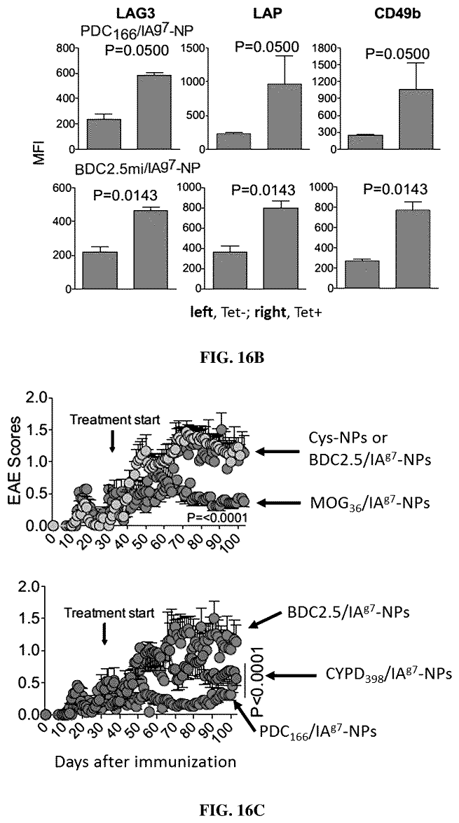

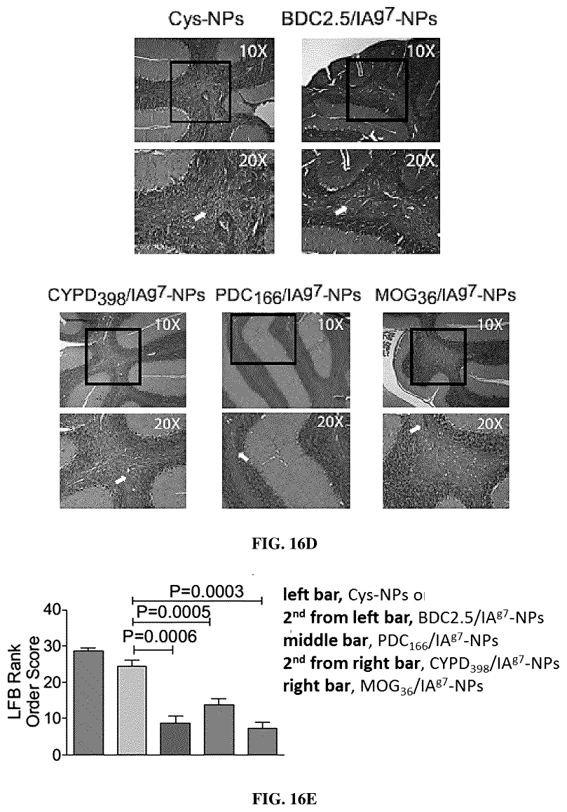

One question unanswered was whether these liver disease-relevant nanomedicines, displaying epitopes from ubiquitous antigens, were liver-specific or, alternatively, also able to blunt extra-hepatic autoimmunity. To investigate this, the ability of PDC-E2₁₆₆₋₁₈₁/IA^{g7}-NPs and CYPD₃₉₈₋₄₁₂/IA^{g7}-NPs (displaying ubiquitous epitopes) vs. BDC2.5/IA^{g7}-NPs (displaying a beta cell-specific epitope) and MOG₃₆₋₅₀/IA^{g7}-NPs (displaying a central nervous system-specific epitope) were compared to see if any could blunt a MOG₃₆₋₅₅-induced relapsing-remitting form of EAE in NOD mice. BDC2.5/IA^{g7}-NPs expanded cognate TR1-like CD4+ T-cells in these animals (in blood and spleen) to a similar extent as they do in non-EAE-affected NOD mice, but these cells were absent from the CNS-draining cervical lymph nodes (CLNs) (FIG. 16B), and had no anti-encephalitogenic activity, as shown in FIG. 16C (oligodendrocytes do not express chromogranin A).

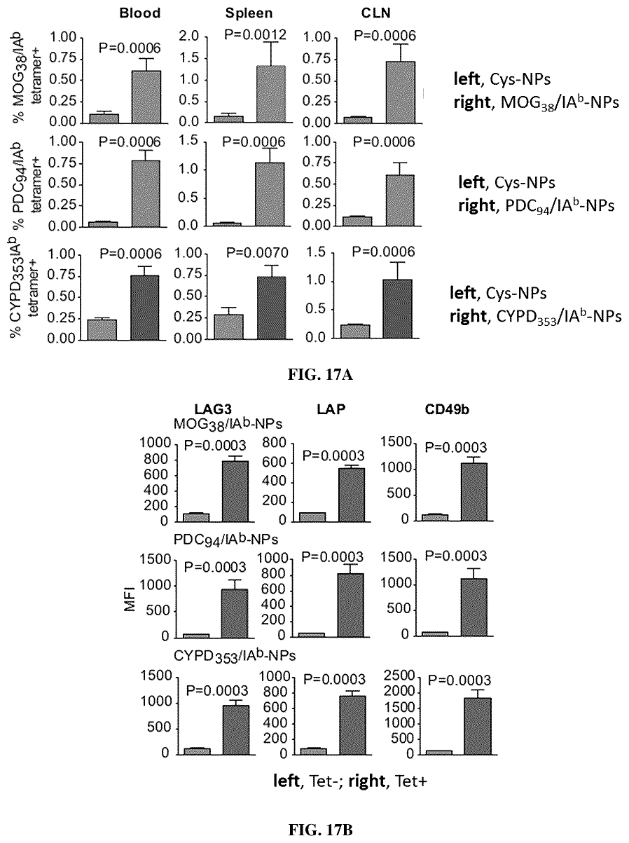

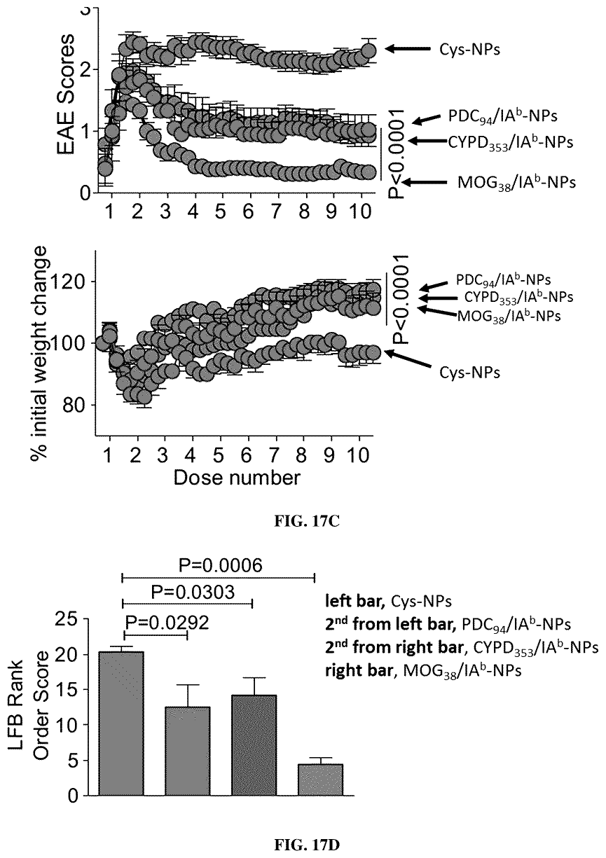

Notably, both CYPD₃₉₈₋₄₁₂/IA^{g7}-NPs and PDC-E2₁₆₆₋₁₈₁/IA^{g7}_NPs also triggered cognate TR1-like cell expansion in these mice (FIG. 16B) but, unlike BDC2.5/IA^{g7}-NPs, suppressed CNS inflammation and demyelination and the progression and severity of EAE relapses (FIGS. 16C, D and 16E). This was associated with an accumulation of cognate TR1-like cells in the CLNs as shown in FIG. 16A. Importantly, these effects were not NOD genetic background-specific, because they were also seen in C57BL/6 mice treated with CYPD and PDC epitope/IA^{b}-based nanomedicines: PDC₉₄₋₁₀₈/IA^{b}-NPs and CYPD₃₅₃₋₃₆₇/IA^{b}-NPs expanded cognate TR1-like CD4+ T-cells in blood, spleen and CLNs (FIGS. 17A and B) and blunted the progression of a chronic form of MOG₃₅₋₅₅-induced EAE in these mice, as compared to Cys-NPs and MOG₃₆₋₄₉/IA^{b}-NPs (negative and positive controls, respectively) as shown in FIGS. 17C and D.

Collectively, these observations suggested that significant levels of two different ubiquitously expressed autoantigens, PDC-E2 and CYPD₂D6, are delivered to local and proximal APCs upon oligodendrocyte cell damage (in EAE), enabling autoreactive CD4+ T-cell priming, cognate TRI cell generation by pMHC-NPs, recruitment and accumulation of these TR1 cells in the CNS-draining lymph nodes, and suppression of local inflammation.

### Example 14—Induction of Beta Cell Apoptosis Renders NOD Mice Responsive to Ubiquitous Autoantigen pMHC-NPs

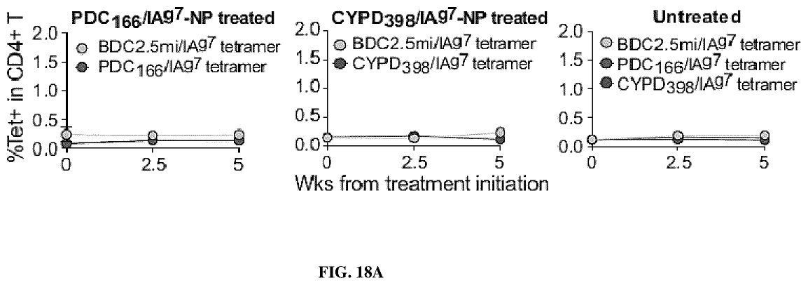

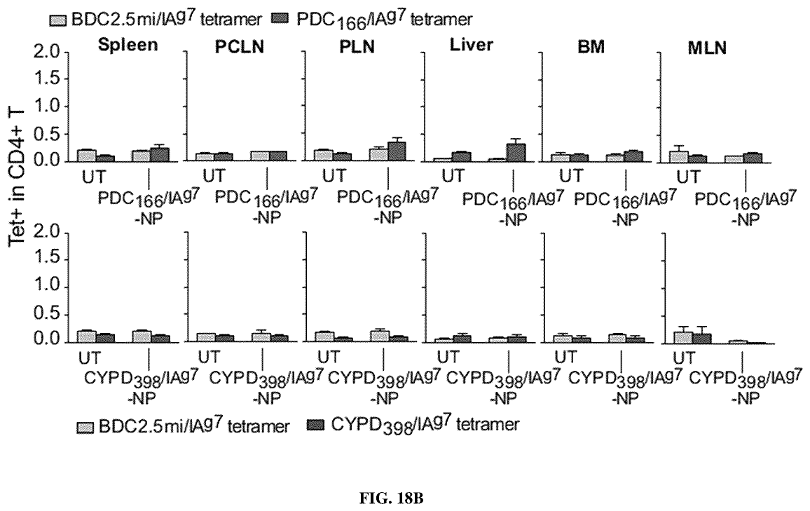

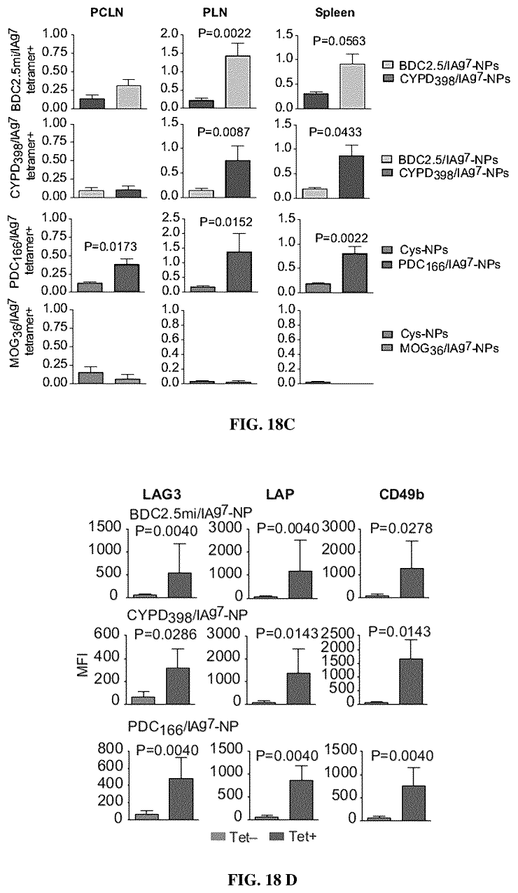

Treatment of pre-diabetic 10 wk-old NOD mice with PDC-E2₁₆₆₋₁₈₁/IA^{g7}-NPs (PBC-relevant) and CYPD₃₉₈₋₄₁₂/IA^{g7}-NPs (AIH-relevant) did not trigger the expansion of cognate TR1-like CD4+ T-cells relative to endogenous BDC2.5/IA^{g7}-specific CD4+ T-cells, as measured with pMHC tetramers, in blood (FIG. 18A) or any of the organs examined, including liver (FIG. 18B).

These results suggested that spontaneous beta cell killing in pre-diabetic NOD mice does not trigger the formation of PDC-E2 or CYPD autoantigen-experienced T-cells capable of responding to the corresponding nanomedicines. Three alternative possibilities could explain these results: 1) these antigens are released from cholangiocytes and hepatocytes (in liver autoimmune diseases) but not from dying beta cells (in T1D); 2) the antigens are released from NOD beta cells, but NOD mice do not export cognate autoreactive T-cells capable of responding to these ubiquitous autoantigens; or 3) the antigens are released from beta cells and the mice harbor cognate autoreactive T-cells, but the antigens are released in amounts insufficient to prime these T-cells.

To distinguish among the above three possibilities, an X-chromosome-linked rat-insulin promoter-driven human diphtheria toxin receptor (hDTR) transgene was expressed in the pancreatic beta cells of non-diabetic female NOD mice. These mice were then treated with DT to simultaneously kill 50% of beta cells (due to X-chromosome inactivation, only 50% of beta cells express the hDTR). We then treated DT-treated and DT-untreated mice with PDC-E2₁₆₆₋₁₈₁/IA^{g7}-NPs, CYPD₃₉₈₋₄₁₂/IA^{g7}_NPs (liver autoimmunity-relevant), BDC2.5/IA^{g7}-NPs (TID-relevant, positive control), MOG₃₆₋₅₀/IA^{g7}-NPs (central nervous system autoimmune disease-specific, negative control) or Cys-coated NPs (negative control) and enumerated the presence of cognate autoreactive T-cells in spleen, liver-draining lymph nodes (portal and celiac LNs, PCLNs) and pancreatic LNs (PLNs), using pMHC tetramers. As expected, MOG₃₆₋₅₀/IA^{g7}-NPs did not trigger the expansion of cognate tetramer+CD4+ T-cells in these mice (MOG is an oligodendrocyte-specific protein that is not expressed in pancreatic beta cells) (FIG. 18C, bottom right panel). In contrast, both PDC-E2₁₆₆₋₁₈₁/IA^{g7}- and CYPD₃₉₈₋₄₁₂/IA^{g7}-NPs triggered the expansion and accumulation of significant numbers of cognate tetramer+CD4+ T-cells expressing the TR1 markers LAG3, LAP and CD49b in the spleen and PLNs of the DT-treated mice, to an extent similar to that seen for BDC2.5/IA^{g7} tetramer+ T-cells in BDC2.5/IA^{g7}-NP-treated animals (FIG. 18C, bottom left and top panels, and FIG. 18D).

These findings corroborate our previous observations that pMHC-NP-induced TR1 cell formation and expansion requires the presence of autoantigen-experienced T-cells, that priming of such cells requires antigen shedding from expressing cell types and that the peripheral repertoire of NOD mice normally harbors naïve CD4+ T-cell specificities targeting ubiquitously-expressed antigens.

### Example 15—Ubiquitous Autoantigen pMHC-NPs Suppress Signs of Psoriasis in a Mouse Model of Induced Psoriasis

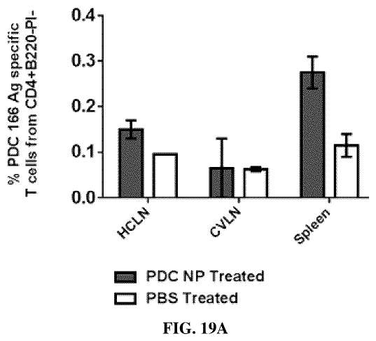

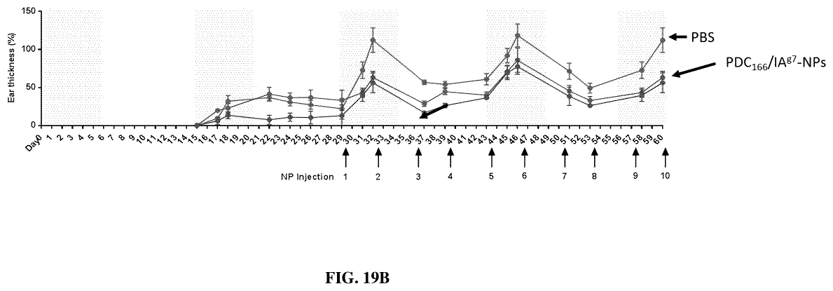

The ability of ubiquitous autoimmune disease relevant pMHC-NPs was tested in a relevant mouse model of psoriasis, the imiquimod (IMQ) induced model. This mouse model is one of the most widely used mouse models in preclinical psoriasis studies. NOD/Ltj mice were treated with 5% topical IMQ for 6 days with 8 day rest intervals. The first application was made to the back of the mice and subsequent applications on the ear. Mice were administered either PDC-E2₁₆₆₋₁₈₁/IA^{g7}- or CYPD₃₉₈₋₄₁₂/IA^{g7}-NPs starting after the second application to the ear. Mice were assayed for symptoms of psoriasis following the PASI (Psoriasis Area and Severity Index) criteria which measures erythema, scaling and skin thickness; and mice were evaluated for expansion of PDC and CYPD tetramer+CD4 T cells in spleen, cervical lymph nodes (CV LN), and hepato-celiac lymph nodes (HC LN). As shown in FIG. 19A PDC-E2₁₆₆₋₁₈₁/IA^{g7} were expanded in this model, indicating that these cells were primed by the induction of inflammation brought on by IMQ treatment. FIG. 19B shows that ear thickness was improved by treatment with PDC-E2₁₆₆₋₁₈₁/IA^{g7}- or CYPD₃₉₈₋₄₁₂/IA^{g7}-NPs, while scaling and erythema were not (not shown). These experiments are preliminary, but indicate that different models of autoimmune/inflammatory disease prime T cells to ubiquitous autoantigens that are able to be modified by ubiquitous autoantigen-relevant pMHC-NPs

### Example 16—Select Methods Utilized Herein

### Mice

NOD/LtJ, BALB/c, C57BL/6, NOD.scid.Il2rg−/−(NSG), NOD.c3c4 and FVB/N.Abcb4−/− (Abcb4 or ATP-binding cassette transporter, sub-family B, member 4) mice were purchased from the Jackson Laboratory (Bar Harbor, ME). IFNγ ARE-Del−/−B6 mice were obtained from H. Young (NIH, Bethesda, MD). NOD.c3c4.scid mice were generated by backcrossing (NOD.c3c4×NOD.scid) F1 mice with NOD.c3c4 mice for five generations, followed by intercrossing of mice heterozygous for the scid mutation and homozygous for the B6 chromosome 3 and 4 intervals from NOD.c3c4 mice. NOD.Abcb4−/− mice were obtained by backcrossing the mutant Abcb4 allele from FVB/N-Abcb4−/−mice onto the NOD/Ltj background for six generations, followed by intercrossing. (NODxB6.IFNg ARE-Del−/−) F1 mice were generated by intercrossing IFNγ ARE-Del−/− and NOD/LtJ mice. NOD.Il10tm1Flv (Tiger) mice were obtained by backcrossing the I110tm1Flv allele from C57BL/6.Il10tm1Flv mice (Jackson Lab) onto the NOD/Ltj background for 10 generations. RIP-DTR.NOD transgenic mice were generated by backcrossing an X-chromosome-linked rat-insulin promoter-driven human diphtheria toxin receptor (RIP-DTR) transgene from transgenic B6 mice into the NOD background for more than 10 generations.

### Cell Lines, Pathogens, and Tumors

CHO-S, BSC-1, MDCK, 293T, B16/F10 and CT26 cell lines were purchased from the ATCC (Manassas, VA). Listeria monocytogenes was obtained from DMX Corporation (Philadelphia, PA).

### Antibodies and Flow Cytometry

FITC, PE, APC, PerCP or biotin-conjugated mAbs against mouse CD4 (RM4-5), CD5 (53-7.3), CD19 (1D3), B220 (RA36B2) and CD49b (HMa2) and streptavidin-PerCP were purchased from BD Biosciences (San Diego, CA). Anti-murine LAG-3 mAb (C9B7W) was purchased from eBioscience (San Diego, CA). Anti-latent-associated-TGF-β (LAP) antibody (TW7-16B4) was from BioLegend (San Diego, CA). PE-conjugated pMHC class II tetramers were produced using biotinylated pMHC monomers. pMHC class II tetramer staining and phenotypic marker analysis were done essentially as described with minor modifications. Briefly, after avidin incubation (15 min at RT), blood leukocytes, and single cell suspensions from spleen, lymph node, liver mononuclear cells, and bone marrow cells were stained first with pMHC tetramer (5 μg ml⁻¹) in FACS buffer (0.05% sodium azide and 1% FBS in PBS) for 60 min at 37° C., and later with FITC-conjugated anti-mouse CD4 (5 ug ml⁻¹) and PerCP-conjugated anti-mouse B220 (2 ug ml⁻¹; as a ‘dump’ channel) for 30 min at 4° C. After washing, cells were fixed (1% paraformaldehyde in PBS) and analyzed with FACScan, FACSaria, or BD LSRII flow cytometers. For phenotypic analyses, the cells were incubated with anti-FcR Abs, and then stained with cell surface marker antibodies diluted 1:100 in FACS buffer (at 4° C. for anti-CD49b and anti-LAP Abs, and at 37° C. for anti-LAG-3 Abs) followed by pMHC tetramer, FITC-conjugated anti-mouse CD4 (5 ug ml⁻¹) and PerCP-conjugated anti-mouse B220. Upon staining, cells were washed, fixed, and analyzed by flow cytometry. FlowJo software was used for all analyses.

NSG-engrafted human T cells were analyzed using the following mAbs: FITC-conjugated anti-CD4 (OKT4, BioLegend), APC-conjugated anti-CD19 (HIB19, BD Biosciences, San Jose, CA), PerCP-conjugated polyclonal goat anti-LAG-3 IgG (R&D Systems, Minneapolis, MN), biotin-conjugated anti-CD49b (AK7, Pierce Antibodies, Thermo Fisher Scientific, Waltham, MA), and eFluor 450-conjugated streptavidin (eBioscience). Briefly, splenocytes and pancreatic lymph node cells were incubated with avidin (0.25 mg ml⁻¹ in FACS buffer) for 30 min at room temperature, washed and stained with tetramer (5 ug ml⁻¹) for 1 h at 37° C., washed, and incubated with FITC-conjugated anti-CD4 (2/100), APC-conjugated anti-CD19 (5/100; used as a ‘dump’ channel), PerCP-conjugated anti-LAG-3 (8/100) and biotin-conjugated anti-CD49b (4/100) at 4° C. for 45 min. After washing, the cells were incubated with eFluor 450-conjugated streptavidin for 30 min at 4° C., washed, fixed in 1% PFA in PBS and cells within the hCD4⁺/hCD19⁻ gate analysed with a FACSCanto II (BD Bioscience).

pMHC Monomers and Peptides

Recombinant pMHC class II monomers were purified from supernatants of CHO-S cells transduced with lentiviruses encoding a monocistronic message in which the peptide-MHCb and MHCa chains of the complex were separated by the ribosome skipping P2A sequence. The peptide was tethered to the amino terminal end of the MHCb chain via a flexible GS linker and the MHCa chains were engineered encode a BirA site, a 6×His tag, a twin strep-tag and a free Cys at their carboxyterminal end. The secreted, self-assembled pMHC class II complexes were purified by sequential nickel and Strep-Tactin® chromatography and used for coating onto NPs or processed for biotinylation and tetramer formation as described above. The epitopes encoded in the murine monomeric constructs were selected based on predicted MHCII-binding capacity using RANKPEP (http://imed.med.ucm.es/cgi-bin/rankpep_mif.cgi) using 7.54 as the threshold score. PDC-E2₁₆₆₋₁₈₁ had a score that fell below the threshold but was selected for experimentation because it is contained within one of the lipoyl-binding domains of PDC-E2, an antigenic target for AMAs. For CYPD and FTCD epitope prediction, we used a second online algorithm (GPS-MBA) (http://mba.biocuckoo.org/) and peptides predicted by both RANKPEP and GPS-MBA were selected for experimentation. hPDC-E2₁₂₂₋₁₃₅, hPDC-E2₂₄₉₋₂₆₂ (both contained within the lipoyl-binding domain of PDC-E2), and hPDC-E2₆₂₉₋₆₄₃ have been described previously. The sequences of the different epitopes are: PDC-E2₁₆₆₋₁₈₁/IA^{g7} (LAEIETDKATIGFEVQ), PDC-E2₈₂₋₉₆/IA^{g7} (EKPQDIEAFKNYTLD), FTCD₅₈₋₇₂/IA^{g7} (VVEGALHAARTASQL), CYPD₃₉₈₋₄₁₂/IA^{g7} (LITNLSSALKDETVW), 2.5 mi/IA^{g7} (AHHPIWARMDA), hPDC-E2₁₂₂₋₁₃₅/DRB4*0101 (GDLIAEVETDKATV), hPDC-E2₂₄₉₋₂₆₂/DRB4*0101 (GDLLAEIETDKATI), and hPDC-E2₆₂₉₋₆₄₃/DRB1*0801 (AQWLAEFRKYLEKPI). Synthetic PDC-E2₁₆₆₋₁₈₁, 2.5 mi, and mMOG₃₆₋₅₅ (EVGWYRSPFSRVVHLYRNGK) peptides were purchased from Genscript (Piscataway, NJ). The amino acid residue numbers for each peptide correspond to those found in the mature form of the corresponding antigens.

Nanoparticles, pMHCII-NP Synthesis, and Purification

pMHCs were coated onto pegylated iron oxide NPs (PFM-NPs), produced as described (2). Briefly, PFM-NPs were produced by thermal decomposition of Fe (acac) 3 in the presence of 2 kD methoxy-PEG-maleimide. The NPs were purified using magnetic (MACS) columns (Miltenyi Biotec, Auburn, CA). Free Cysteines (controls) or pMHCs, carrying a free carboxyterminal Cys, were conjugated to the maleimide-functionalized PFMs in 40 mM phosphate buffer, pH 6.0, containing 2 mM EDTA, 150 mM NaCl overnight at room temperature. The pMHC-conjugated NPs were separated from free pMHC using magnetic columns, sterilized by filtration through 0.2 μm filters and stored in water or PBS at 4° C. Quality control was done using transmission electron microscopy, dynamic light scattering, and native and denaturing gel electrophoresis. pMHC content was measured using Bradford assay (Thermo Fisher Scientific) and SDS-PAGE.

### Generation of FTCD-Expressing Adenovirus

A replication-deficient adenovirus expressing human formiminotransferase cyclodeaminase (Ad-hFTCD) (a target autoantigen in AIH Type 2) was generated by cloning the hFTCD DNA sequence directly into Adeno-X Adenoviral System 3 (CMV) using In-Fusion® HD cloning technology and Stellar Competent cells (Clontech, Mountain View, CA). Cloned Ad-FTCD was amplified in Ad-293 T cells and purified using Adeno-X Maxi Purification Kit (Clontech). The viral titer was measured using the End-point Dilution Assay or Adeno-X Rapid Titer Kit (Clontech).

### Ursodeoxycholic Acid Treatment

Cohorts of 5-6 or 24 wk-old male and/or female NOD.c3c4 mice were left untreated, fed a diet supplemented with 0.5% UDCA (BOC Sciences, Upton, NY; TestDiet, Richmond, IN), or treated with pMHCII-NPs for 14 or 9 wk, respectively, and sacrificed for pMHCII tetramer staining, PBC scoring and biochemical testing.

pMHCII-NP Therapy in NOD.c3c4, (NOD×B6.IFNg ARE-Del−/−) F1 and NOD/Ltj Mice

Cohorts of 15 wk-old male and/or female NOD.c3c4 mice with established PBC were left untreated or treated with 20 mg of pMHCII-NPs or Cys-NPs (i.v.) twice weekly for 9 wk unless indicated otherwise. Liver disease scoring involved macroscopic evaluation of cyst content (0-5), liver weight and CBD diameter (0-4), as well as microscopic evaluation of bile duct involvement (0-4), bile duct proliferation (0-4) and mononuclear cell infiltration (0-4), essentially as described (23). In other experiments, treatment was initiated at the peak of disease (24 wk of age) and given twice a week for 14-20 wk. Intermittent treatment involved treating mice twice a wk from 15 to 24 wk of age, then withdrawing treatment until the percentages of tetramer+ cells dropped to ~50% of the levels seen at treatment withdrawal (measurements in peripheral blood were done once every two wk), re-treating mice twice a wk until the percentages of tetramer+ cells reached original values, and repeating this cycle until 50 wk of age.

In in vivo cytokine blocking experiments, mAbs against HRPN (rlgG1), IL-10 (JES5-2A5) or TGF-β (1D11) (BioXcell, West Lebanon, NH) were given i.p. twice a week at 500 mg per dose for 2 wk, followed by 200 mg per dose for 7 additional wk. Mice were randomized into cytokine-neutralizing mAb-treatment (anti-IL-10 or -TGFβ) or HRPN rat-IgG1 groups.

In experiments involving (NOD×B6.IFNg ARE-Del′-) F1 mice, 10-wk-old male and female mice were treated for 5-6 wk. Histopathologic scoring in the liver was performed as described. Briefly, severity scores were obtained by scoring portal inflammation, lobular inflammation and granuloma formation from 0-4, and bile duct damage from 0-2. The extent of portal inflammation and bile duct damage were scored from 0-4 based on the ratio between affected vs unaffected area. The extent of lobular inflammation and granuloma formation were scored from 0-4 based on number of lesions per specimen. The severity of fibrosis was scored on a 0-6 scale.

Studies using NOD mice involved treating cohorts of 10-wk-old pre-diabetic female NOD/Ltj mice with 20 mg of pMHCII-NPs or Cys-NPs i.v. twice weekly for 5 wk.

pMHCII-NP Therapy for EAE in NOD.c3c4 Mice

Male and female 12-14 wk-old NOD.c3c4 mice were immunized with 250 mg of pMOG₃₆₋₅₅ in CFA s.c. on either side of the flank region above the base of the tail under isofluorane anesthesia. The mice received 350 ng of Pertussis toxin i.v. on days 0 and 2 relative to peptide immunization. Mice were weighed and scored on day 0 and then daily starting on day 14 after immunization, and scores were plotted on a 5-point scale. On day 32 of immunization, all the mice having scores <3 were randomized into treatment with Cys-NPs or pMHCII-NPs. All mice were treated twice a week for 9 wk. Scoring of mice for signs of PBC was done as described above.

pMHCII-NP Therapy for PSC in NOD.Abcb4−/− Mice

Cohorts of 5-6 wk-old male and/or female NOD.Abcb4′ mice with established PSC were treated with 20 mg of pMHCII-NPs or Cys-NPs i.v. twice weekly for 5-6 wk. Histopathologic lesions were graded using the Ishak scoring system, which evaluates both fibrosis (0-6) as well as necroinflammatory sequelae of biliary cholangitis, including interface hepatitis (0-4), confluent necrosis (0-6), lobular inflammation (0-4) and portal inflammation (0-4).

pMHCII-NP Therapy for AIH in NOD Mice

AIH was induced by infecting 5-6 wk-old female NOD/Ltj mice with an adenovirus encoding human FTCD (Ad-hFTCD, 1010 plaque forming units (PFU) i.v.). Four wks later, cohorts of mice with established AIH were treated with 20 mg of pMHCII-NP s or Cys-NPs (i.v.) twice weekly for 5-6 wk. Histopathological scoring was done using the Ishak scale as above.

pMHCII-NP Therapy in Human PBMC-Reconstituted NSG Hosts

PBMCs from HLA-DRB4*0101+PBC patients (recruited under informed consent approved by the Institutional Review Board at Hospital Clinic) were depleted of CD8+ T-cells using anti-CD8 mAb-coated magnetic beads (Miltenyi Biotech, Auburn, CA) and injected i.v. (2×10⁷) into 8-10 wk-old NSG hosts. Mice were treated with 30-40 mg pMHC-NPs starting on day 5 after PBMC transfusion, twice a wk for 5 consecutive wks, or left untreated. Therapy-induced expansion of cognate CD4+ T-cells was measured in liver, peripheral LNs, spleen and bone marrow (not shown). A mouse was considered a responder if the percentage of tetramer+ T-cells in at least two different organs were higher than the mean±10 standard deviation values seen in untreated hosts.

### Evaluation of General Adaptive Immunity

pMHCII-NP-treated and untreated female mice were injected i.v. with 2×10⁶ PFU of recombinant Vaccinia Virus (rVV) and sacrificed on days 4 and 14 after infection. Samples were processed for pMHCII tetramer staining and rVV titer measurements. Briefly, both ovaries were collected in DMEM containing 2% FBS, homogenized, freeze-thawed 3 times followed by sonication (3 rounds, 20 sec each). Serial dilutions of the lysates were added to confluent BSC-1 cell cultures at 37° C. for 1h, washed twice with serum free DMEM and then overlaid with DMEM containing 2% FCS and 0.4% carboxymethyl cellulose (CMC; Sigma, Saint Louis, MO). On day 3, the overlay was discarded, and the cell layers were stained with crystal violet to count the number of plaques.

To evaluate cellular responses to Influenza infection, pMHCII-NP-treated and untreated mice were first primed i.p. with the HKx31 (H3N2) strain at 10⁶ EID₅₀ per mouse. One cohort of mice was sacrificed 7d after priming and processed for tetramer staining to confirm presence of pMHC-NP-specific TR1-like cells during priming. Other cohorts of primed mice were re-infected 30 d later with an intranasal dose of PR8 virus, a lethal H1N1 strain of Influenza (8×10⁴ EID₅₀ per mouse), under anesthesia. PR8-challenged mice were weighed daily and scored clinically from 0-4 based on the extent of ruffled fur, reduced motility, huddled appearance, and rapid and/or labored breathing. Mice were sacrificed 7d later and processed for tetramer staining and influenza titer measurement. Briefly, lungs were collected in serum free DMEM, homogenized and freeze-thawed 3 times. Serial dilutions of the lysates were added to confluent MDCK cell cultures at RT for 1 h and washed. Cultures were then overlaid with DMEM containing 0.4% CMC for 2-3 days, washed, fixed and stained with crystal violet to count the number of plaques.

Cellular immunity to intracellular bacteria was determined by infecting pMHCII-NP-treated and untreated mice i.v. with 103 colony forming units (cfu) of Listeria monocytogenes. Mice were sacrificed 7d or 14d after infection and samples processed for tetramer staining and bacterial load measurements. Briefly, spleen and liver were cut into several pieces, weighed and homogenized in PBS containing 0.35% Triton X-100. Serial dilutions of the lysates were then plated onto Bovine Heart Infusion agar containing 5 mg/ml erythromycin, incubated for 24-48 h at 37° C. and the number of colonies counted.

Cellular immunity to liver metastatic tumors was ascertained upon intra-splenic injections of B16/F10 melanoma and CT26 colon carcinoma tumors into syngeneic (C57BL/6J or Balb/c, respectively) or allogeneic hosts (pMHC-NP-treated or untreated NOD.c3c4 mice). A small incision was made in the abdomen, under isofluorane inhalational anesthesia, to partially expose the spleen. Tumor cells (0.2×10⁶ and 0.1×10⁶ for B16/F10 and CT26, respectively, in 100 mL of PBS) were injected slowly for 1 min into the exposed spleen. Ten minutes later, the spleen was removed and the peritoneal and skin layers sutured. pMHCII-NP therapy was resumed within 5-7d after surgery and continued until the end of follow-up. Mice were monitored for up to 19-21d and euthanized for tetramer staining, PBC scoring, and tumor burden measurements. In B16/F10-injected mice, tumor burden was assessed by measuring liver weight and counting the number of metastases, easily distinguishable from liver parenchyma. In CT26-injected animals, tumor burden was scored histologically by measuring the hepatic area (HPA) occupied by metastatic tumors.

To evaluate humoral immunity, pMHCII-NP-treated and untreated mice were immunized i.p. with 100 mg of DNP-KLH (Alpha Diagnostic International, San Antonio, TX) in CFA and boosted again 3 wk later. Mice were sacrificed 10 d later, to measure serum anti-DNP antibody titers using an anti-DNP Ig ELISA Kit (Alpha Diagnostic International).

### Cytokine Secretion Assays

Splenic and portal/celiac lymph node (PCLN) cell suspensions from pMHCII-NP-treated mice were enriched for CD4+ T-cells depleting CD19+ B-cells (EasySep™ Mouse CD19 Positive Selection Kit, Stem Cell Technologies, Vancouver, BC) and CD8+ T-cells (CD8 Magnetic Particles, BD Biosciences). Cells were stained with pMHCII tetramers and sorted by flow cytometry. The sorted cells (2-3×10⁴) were challenged with bone marrow-derived DCs (2×10⁴) pulsed with 2 ug ml⁻¹ peptide. Forty-eight hours later, supernatants were harvested for measurement of cytokine content via Luminex®.

To ascertain whether pMHCII-NP therapy promoted the recruitment/formation of IL-10-secreting B-cells, mesenteric LNs, PCLNs and liver cell suspensions were enriched for B-cells using a CD19 enrichment kit (Stem Cell Technologies). The cells (2-3×10⁵ in 200 mL/well) were stimulated in duplicate with LPS (1 ug ml⁻¹, Sigma) for 24 h in RPMI-1640 media containing 10% FCS. The levels of IL-10 in the supernatants were measured via Luminex®.

### Isolation and In Vitro Stimulation of Lymph Node CD11b+ Cells and Liver Kupffer Cells

CD11b+ cells from LNs were obtained by digestion in collagenase D (1.25 ug mL-1) and DNAse I (0.1 ug mL⁻¹) for 15 min at 37° C., washed, incubated with anti-FcR Abs, and purified using anti-CD11b mAb-coated magnetic beads (BD Biosciences). The purified cells (2-3×10⁵ in 200 mL/well) were stimulated with LPS (2 ug ml⁻¹) for 3 days, and the supernatants analyzed for cytokine content using a Luminex® multiplex cytokine assay.

To isolate Kupffer cells (KCs), livers from treated and untreated mice were minced and digested in 15 ml of 0.05% collagenase solution in HBSS for 20-30 min at 37° C. The resulting cell suspension was filtered through a nylon mesh (0.7 μm) and centrifuged at 50×g for 3 min at 4° C., to remove tissue debris and hepatocytes. Cells in the supernatant were pelleted by centrifugation at 300×g for 5 min at 4° C. The cell pellet, mainly composed of non-parenchymal liver immune cells, KCs, sinusoidal endothelial cells and stellate cells, was re-suspended in 33% Percoll® solution and centrifuged at 350×g for 30 min to isolate mononuclear cells. The pellets were re-suspended in DMEM containing 10% FCS (5×10⁶ cells ml⁻¹) and plated in 6-well plate at 1-3×10⁷ cells/well and incubated for 2-3 h in a 5% CO₂ atmosphere at 37° C. Non-adherent cells were removed by gentle washing with PBS. The adherent fraction (enriched for KCs) was harvested by trypsin digestion (5 min, 0.25% trypsin). The resulting cell suspension was plated in 96 well plates at 1-2×10⁵/200 mL/well and stimulated with LPS (2 mg ml⁻¹) for 3d. The supernatants were analyzed for cytokine content using a Luminex® multiplex cytokine assay.

### Adoptive Transfer of Suppression

Splenic CD4+ T-cells (107) from untreated mice or mice treated with 12 doses of PDC-E2166-181/IA^{g7}-NPs were adoptively transferred (i.v.) into 10-14 wk-old, sex-matched NOD.c3c4.scid hosts. One day later, the recipients were adoptively transferred with 4×10⁷ whole splenocytes from sex-matched NOD.c3c4 donor mice with established PBC (>35 wk-old). One of the cohorts of mice transfused with CD4+ T-cells from pMHCII-NP-treated donors was further treated with 12 doses of PDC-E2₁₆₆₋₁₈₁/IA^{g7}-NPs. The recipients were sacrificed 6wk later for tetramer staining and PBC scoring.

### In Vivo Breg Induction Assay

Splenic B-cells from NOD.Il10fmIFlv (Tiger) mice were enriched using an EasySep Mouse B-cell Isolation Kit (Stem Cell Technologies) and pulsed with BDC2.5 mi or PDC₁₆₆₋₁₈₁ peptides (10 ug ml⁻¹) for 2 h at 37° C. The peptide-pulsed B-cells were washed twice with PBS, labeled with PKH26 (Sigma) and transfused (3×10⁶) into pMHC-NP-treated or untreated mice. The hosts were killed 7 d later and their spleens, MLNs, PCLNs and liver mononuclear cells were labeled with anti-B220-APC and biotinylated anti-CD1d or anti-CD5 mAbs followed by Streptavidin-PerCP. PKH26+B-cells were analyzed for presence of eGFP+/CD1d^{high} and eGFP+/CD5+ cells by flow cytometry.

### Histology and Immunohistochemistry

Livers were fixed in 10% formalin for 2d, embedded in paraffin, cut into 5 μm sections and stained with H&E or Picrosirius Red. For immunohistochemistry, liver tissues were embedded in Tissue-Tek OCT, sectioned into 30 μm cryosections and stored on slides at −80° C. Slides were fixed in chilled acetone, washed with PBS, treated with a 1:10 dilution of 30% H₂O₂ in PBS, washed with PBS, blocked with 10% normal goat serum in PBS, washed again, and stained with anti-mouse CD4 (GK1.5) or CD8 (Lyt-2) antibodies (1.5h, 4° C.). After washing, the slides were stained with a biotinylated goat anti-rat secondary antibody (1:200 dilution), incubated with Horseradish Peroxidase (HRP)-conjugated streptavidin, followed by 3,3-diaminobenzidine (DAB) substrate. Slides were counterstained with hematoxylin before mounting.

### ALT and TBA Assays

Alanine aminotransferase (ALT) levels in serum were determined using a kit from Thermo Fisher Scientific following the manufacturer's protocol. Briefly, serum samples were mixed with pre-warmed (37° C.) Infinity™ ALT (GPT) Liquid Stable Reagent at 1:10 ratio and OD readings were taken for 3 min at 1 min intervals in a nanodrop at a 340 nm wavelength, 37° C. The slope was calculated by plotting absorbance vs. time using linear regression and multiplied with a factor to obtain ALT levels in serum (U/L) as described in the kit. Serum total bile acid (TBA) levels were analyzed using a TBA Enzymatic Cycling Assay Kit (Diazyme, Poway, CA) following a modified manufacturer's protocol as described.

### Anti-Nuclear and Anti-Mitochondrial Autoantibody Measurements

Presence of anti-nuclear autoantibodies (ANAs) in serum was ascertained using NOVA Lite®HEp-2 Slides kit (Inova Diagnostics, San Diego, CA). A semi-quantitative approach was followed to measure ANA titers. Briefly, serum samples were serially diluted in PBS (at 1:160, 1:320, 1:640, 1:1280 and 1:2560) and then added to pre-fixed Hep-2 substrate slides, washed, stained with FITC-conjugated goat anti-mouse IgG in PBS containing 5% normal donkey serum (1:200 dilution), washed, mounted and read under a fluorescent microscope.

Serum levels of anti-mitochondrial PDC-E2 antibodies were determined via ELISA. Briefly, ELISA plates were coated with PDC-E2 protein (5 ug ml⁻¹, 100 mL) (SurModics Inc., Eden Prairie, MN) overnight at RT. Plates were washed, blocked using 3% dry skim milk in PBS (pH 7.4, 150 ml), and incubated with serially-diluted serum samples (100 ml, at 1:250 dilutions prepared using reagent diluent) for 2 h at RT. Wells were washed and incubated with 100 mL of HRP-conjugated anti-mouse IgG (1:2000 in reagent diluent) for 2 h at RT, and washed. Finally, wells were incubated in the dark with 100 mL of DAB substrate for 20 min at RT. Upon stopping the enzymatic reaction with 50 mL 2N H₂SO₄, the absorption was measured at a 450 nm wavelength using an ELISA plate reader. The Positive Antibody Activity (PAA) levels were calculated by calculating the mean OD±2 SD of the control NOD serum samples (positive index) and by dividing the OD values corresponding to NOD.c3c4 serum samples by the positive index, whereby values >1.0 correspond to PAA.

### Statistical Analyses

Unless specified, sample size values mentioned in the figure legends correspond to the total number of mice examined, pooled from different experiments. Data were compared in GraphPad Prism 6 using Mann-Whitney U-test, Chi-Square, Log-Rank (Mantel-Cox), Pearson correlation, two-way ANOVA or multiple t test analyses using the Holm-Sidak correction. P values <0.05 were considered statistically significant. Only statistically significant P values are displayed on Figures.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### Embodiments of the Current Description

The following embodiments provide a non-exhaustive list of specific compositions and uses of the current invention.

1. In a certain aspect described herein is a composition comprising:
a) a plurality of antigen-major histocompatibility complexes (MHCs), each antigen-MHC of the plurality comprising a ubiquitous autoantigen, that is not a tissue specific antigen, associated with a binding groove of an MHC molecule; and
b) a nanoparticle core possessing a diameter of between 1 and 100 nanometers;
wherein the antigen-MHCs are coupled to the nanoparticle core.

2. An embodiment of the composition of embodiment 1, wherein the MHC molecule is an MHC class II molecule.

3. An embodiment of the composition of embodiment 1, wherein the nanoparticle core is a metal or metal oxide.

4. An embodiment of the composition of embodiment 3, wherein the metal is iron.

5. An embodiment of the composition of embodiment 3, wherein the metal oxide is iron oxide.

6. An embodiment of the composition of any one of embodiments 1 to 5, wherein the diameter is between about 5 nanometers and about 50 nanometers.

7. An embodiment of the composition of embodiment 6, wherein the diameter is between about 5 nanometers and about 25 nanometers.

8. An embodiment of the composition of any one of embodiments 1 to 7, wherein the plurality of antigen-MHCs is coupled to the nanoparticle core at an antigen-MHC to nanoparticle core ratio of at least 10:1.

9. An embodiment of the composition of any one of embodiments 1 to 8, wherein the plurality of antigen-MHCs is coupled to the nanoparticle core at an antigen-MHC to nanoparticle core ratio of no more than 150:1.

10. An embodiment of the composition of any one of embodiments 1 to 9, wherein the plurality of antigen-MHCs is coupled to the nanoparticle core at a density from about 0.4 to about 13 antigen-MHCs per 100 nm² of nanoparticle core surface area.

11. An embodiment of the composition of any one of embodiments 1 to 10, wherein the antigen-MHCs are covalently coupled to the nanoparticle core.

12. An embodiment of the composition of any one of embodiments 1 to 11, wherein the antigen-MHCs are covalently coupled to the nanoparticle core by a polyethylene glycol (PEG) linker having a mass of less than about 5 kilodaltons.

13. An embodiment of the composition of any one of embodiments 1 to 12, wherein the nanoparticle core further comprises a biocompatible coating.

14. An embodiment of the composition of any one of embodiments 1 to 13, wherein the ubiquitous autoantigen comprises a polypeptide derived from a protein that at steady-state exists in or on an intracellular compartment.

15. An embodiment of the composition of embodiment 14, wherein the intracellular compartment is cytosol, mitochondria, Golgi apparatus, endoplasmic reticulum, nucleus, or plasma membrane.

16. An embodiment of the composition of embodiment 14, wherein the intracellular compartment is a mitochondrion.

17. An embodiment of the composition of any one of embodiments 1 to 13, wherein the ubiquitous autoantigen is pyruvate dehydrogenase complex-E2 component (PDC-E2).

18. The composition of any one of embodiments 1 to 13, wherein the ubiquitous autoantigen is Cytochrome P450 2D6 (CYP2D6).

19. An embodiment of the composition of any one of embodiments 1 to 13, wherein the ubiquitous autoantigen is actin (ACTB).

20. An embodiment of the composition of any one of embodiments 1 to 13, wherein the ubiquitous autoantigen is soluble liver antigen (SLA).

21. An embodiment of the composition of any one of embodiments 1 to 13, wherein the ubiquitous autoantigen is formimidoyltransferase-cyclodeaminase (FTCD).

22. An embodiment of the composition of any one of embodiments 1 to 13, wherein the ubiquitous autoantigen is myeloperoxidase (MPO).

23. An embodiment of the composition of embodiment 17, wherein the ubiquitous autoantigen is selected from the group consisting of: PDC-E2₃₅₃₋₃₆₇; PDC-E2₇₂₋₈₆; PDC-E2₄₂₂₋₄₃₆; PDC-E2₃₅₃₋₃₆₇; PDC-E2₈₀₋₉₄; PDC-E2₅₃₅₋₅₄₉; PDC-E2₆₂₉₋₆₄₈; PDC-E2₁₂₂₋₁₃₅ PDC-E2₂₄₉₋₂₆₃; and PDC-E2₂₄₉₋₂₆₃.

24. An embodiment of the composition of embodiment 17, wherein the ubiquitous autoantigen is selected from the group consisting of: PDC-E2₄₂₂₋₄₃₆ and PDC-E2₈₀₋₉₄.

25. An embodiment of the composition of embodiment 18, wherein the ubiquitous autoantigen is selected from the group consisting of: CYP2D6₂₈₄₋₂₉₈; CYP2D6₂₈₉₋₃₀₃; CYP2D6₃₁₈₋₃₃₂; CYP2D6₃₁₃₋₃₃₂; CYP2D6₃₉₃₋₄₁₂; CYP2D6₁₉₂₋₂₀₆; CYP2D6₅₋₁₉; CYP2D6₂₉₃₋₃₀₇; CYP2D6₂₁₉₋₂₃₃; CYP2D6₂₃₇₋₂₅₁; CYP2D6₁₅₋₂₉; CYP2D6₂₃₅₋₂₄₉; CYP2D6₃₁₇₋₃₃₁; CYP2D6₂₉₃₋₃₀₇; CYP2D6₄₂₈₋₄₄₂; CYP2D6₂₃₇₋₂₅₁; CYP2D6₁₄₋₂₈; CYP2D6₁₉₉₋₂₁₃; CYP2D6₄₅₀₋₄₆₄; CYP2D6₃₀₁₋₃₁₅; CYP2D6₄₅₂₋₄₆₆; CYP2D6₅₉₋₇₃; CYP2D6₁₃₀₋₁₄₄; CYP2D6₁₉₃₋₂₁₂; CYP2D6₃₀₅₋₃₂₄; and CYP2D6₁₅₋₂₉.

26. An embodiment of the composition of embodiment 19, wherein the ubiquitous autoantigen is selected from the group consisting of: ACTB₂₀₂₋₂₁₆; ACTB₁₇₀₋₁₈₄; ACTB₂₄₅₋₂₅₉; ACTB₁₈₇₋₂₀₁; ACTB₁₇₂₋₁₈₆; ACTB₁₃₁₋₁₄₅; ACTB₁₃₁₋₁₄₅; ACTB₁₇₁₋₁₈₅; ACTB₁₂₉₋₁₄₃; ACTB₁₆₄₋₁₇₈; ACTB₂₅₋₃₉; and ACTB₃₂₃₋₃₃₇.

27. An embodiment of the composition of embodiment 19, wherein the ubiquitous autoantigen is selected from the group consisting of: ACTB₁₄₆₋₁₆₀; ACTB₁₈₋₃₂; and ACTB₁₇₁₋₁₈₅.

28. An embodiment of the composition of embodiment 20, wherein the ubiquitous autoantigen is selected from the group consisting of: SLA₃₃₄₋₃₄₈; SLA₁₉₆₋₂₁₀; SLA₁₁₅₋₁₂₉; SLA₃₇₃₋₃₈₆; SLA₁₈₆₋₁₉₇; SLA₃₄₂₋₂₅₆; SLA₁₁₀₋₁₂₄; SLA₂₉₉₋₃₁₃; SLA₄₉₋₆₃; SLA₂₆₀₋₂₇₄; SLA₁₁₉₋₁₃₃; SLA₈₆₋₁₀₀; SLA₂₆₋₄₀; SLA₃₃₁₋₃₄₅; SLA₃₁₇₋₃₃₁; SLA₁₇₁₋₁₈₅; SLA₄₁₇₋₄₃₁; SLA₃₅₉₋₃₇₃; SLA₂₁₅₋₂₂₉; and SLA₁₁₁₋₁₂₅.

29. An embodiment of the composition of embodiment 21, wherein the ubiquitous autoantigen is selected from the group consisting of: FTCD₄₃₉₋₄₅₃; FTCD₃₈₁₋₃₉₅; FTCD₂₉₇₋₃₁₁; FTCD₅₂₅₋₅₃₉; FTCD₂₁₈₋₂₃₂; FTCD₄₉₅₋₅₀₉; FTCD₂₆₂₋₂₇₆; FTCD₃₀₀₋₃₁₄; FTCD₂₅₉₋₂₇₃; FTCD₄₉₀₋₅₀₄; FTCD₃₈₉₋₄₀₃; and FTCD₂₉₅₋₃₀₉.

30. An embodiment of the composition of embodiment 21, wherein the ubiquitous autoantigen is selected from the group consisting of: FTCD₂₇₁₋₂₈₅; FTCD₄₉₈₋₅₁₂; and FTCD₃₀₁₋₃₁₅.

31. An embodiment of the composition of embodiment 22, wherein the ubiquitous autoantigen is selected from the group consisting of: MPO₃₂₂₋₃₃₆; MPO₇₁₄₋₇₂₈; MPO₆₁₇₋₆₃₁; MPO₅₀₄₋₅₁₈; MPO₄₆₂₋₄₇₆; MPO₆₁₇₋₆₃₁; MPO₄₄₄₋₄₅₈; MPO₆₈₉₋₇₀₃; MPO₂₄₈₋₂₆₂; MPO₅₁₁₋₅₂₅; MPO₉₇₋₁₁₁; and MPO₆₁₆₋₆₃₀.

32. An embodiment of the composition of any one of embodiments 1 to 31, further comprising a second plurality of antigen-major histocompatibility complexes (MHCs) coupled to the nanoparticle core, each antigen-MHC of the second plurality comprising an antigen.

33. An embodiment of the composition of embodiment 32, wherein the antigen of the second plurality of antigen-major histocompatibility complexes (MHCs) is a second ubiquitous autoantigen.

34. An embodiment of the composition of embodiment 32, wherein the second ubiquitous autoantigen comprises a polypeptide derived from a protein that at steady-state exists in or on an intracellular compartment.

35. An embodiment of the composition of embodiment 34, wherein the intracellular compartment is cytosol, mitochondria, Golgi apparatus, endoplasmic reticulum, nucleus, or plasma membrane.

36. An embodiment of the composition of embodiment 34, wherein the intracellular compartment is a mitochondrion.

37. An embodiment of the composition of embodiment 34, wherein the second ubiquitous autoantigen is selected from the group consisting of: PDC-E2₃₅₃₋₃₆₇; PDC-E2₇₂₋₈₆; PDC-E2₄₂₂₋₄₃₆; PDC-E2₃₅₃₋₃₆₇; PDC-E2₈₀₋₉₄; PDC-E2₅₃₅₋₅₄₉; PDC-E2₆₂₉₋₆₄₈; PDC-E2₁₂₂₋₁₃₅ PDC-E2₂₄₉₋₂₆₃; and PDC-E2₂₄₉₋₂₆₃.

38. An embodiment of the composition of embodiment 34, wherein the second ubiquitous autoantigen is selected from the group consisting of: PDC-E2₄₂₂₋₄₃₆ and PDC-E2₈₀₋₉₄.

39. An embodiment of the composition of embodiment 34, wherein the second ubiquitous autoantigen is selected from the group consisting of: CYP2D6₂₈₄₋₂₉₈; CYP2D6₂₈₉₋₃₀₃; CYP2D6₃₁₈₋₃₃₂; CYP2D6₃₁₃₋₃₃₂; CYP2D6₃₉₃₋₄₁₂; CYP2D6₁₉₂₋₂₀₆; CYP2D6₅₋₁₉; CYP2D6₂₉₃₋₃₀₇; CYP2D6₂₁₉₋₂₃₃; CYP2D6₂₃₇₋₂₅₁; CYP2D6₁₅₋₂₉; CYP2D6₂₃₅₋₂₄₉; CYP2D6₃₁₇₋₃₃₁; CYP2D6₂₉₃₋₃₀₇; CYP2D6₄₂₈₋₄₄₂; CYP2D6₂₃₇₋₂₅₁; CYP2D6₁₄₋₂₈; CYP2D6₁₉₉₋₂₁₃; CYP2D6₄₅₀₋₄₆₄; CYP2D6₃₀₁₋₃₁₅; CYP2D6₄₅₂₋₄₆₆; CYP2D6₅₉₋₇₃; CYP2D6₁₃₀₋₁₄₄; CYP2D6₁₉₃₋₂₁₂; CYP2D6₃₀₅₋₃₂₄; and CYP2D6₁₅₋₂₉.

40. An embodiment of the composition of embodiment 34, wherein the second ubiquitous autoantigen is selected from the group consisting of: ACTB₂₀₂₋₂₁₆; ACTB₁₇₀₋₁₈₄; ACTB₂₄₅₋₂₅₉; ACTB₁₈₇₋₂₀₁; ACTB₁₇₂₋₁₈₆; ACTB₁₃₁₋₁₄₅; ACTB₁₃₁₋₁₄₅; ACTB₁₇₁₋₁₈₅; ACTB₁₂₉₋₁₄₃; ACTB₁₆₄₋₁₇₈; ACTB₂₅₋₃₉; and ACTB₃₂₃₋₃₃₇.

41. An embodiment of the composition of embodiment 34, wherein the second ubiquitous autoantigen is selected from the group consisting of: ACTB₁₄₆₋₁₆₀; ACTB₁₈₋₃₂; and ACTB₁₇₁₋₁₈₅.

42. An embodiment of the composition of embodiment 34, wherein the second ubiquitous autoantigen is selected from the group consisting of: SLA₃₃₄₋₃₄₈; SLA₁₉₆₋₂₁₀; SLA₁₁₅₋₁₂₉; SLA₃₇₃₋₃₈₆; SLA₁₈₆₋₁₉₇; SLA₃₄₂₋₂₅₆; SLA₁₁₀₋₁₂₄; SLA₂₉₉₋₃₁₃; SLA₄₉₋₆₃; SLA₂₆₀₋₂₇₄; SLA₁₁₉₋₁₃₃; SLA₈₆₋₁₀₀; SLA₂₆₋₄₀; SLA₃₃₁₋₃₄₅; SLA₃₁₇₋₃₃₁; SLA₁₇₁₋₁₈₅; SLA₄₁₇₋₄₃₁; SLA₃₅₉₋₃₇₃; SLA₂₁₅₋₂₂₉; and SLA₁₁₁₋₁₂₅.

43. An embodiment of the composition of embodiment 34, wherein the second ubiquitous autoantigen is selected from the group consisting of: FTCD₄₃₉₋₄₅₃; FTCD₃₈₁₋₃₉₅; FTCD₂₉₇₋₃₁₁; FTCD₅₂₅₋₅₃₉; FTCD₂₁₈₋₂₃₂; FTCD₄₉₅₋₅₀₉; FTCD₂₆₂₋₂₇₆; FTCD₃₀₀₋₃₁₄; FTCD₂₅₉₋₂₇₃; FTCD₄₉₀₋₅₀₄; FTCD₃₈₉₋₄₀₃; and FTCD₂₉₅₋₃₀₉.

44. An embodiment of the composition of embodiment 34, wherein the second ubiquitous autoantigen is selected from the group consisting of: FTCD₂₇₁₋₂₈₅; FTCD₄₉₈₋₅₁₂; and FTCD₃₀₁₋₃₁₅.

45. An embodiment of the composition of embodiment 34, wherein the second ubiquitous autoantigen is selected from the group consisting of: MPO₃₂₂₋₃₃₆; MPO₇₁₄₋₇₂₈; MPO₆₁₇₋₆₃₁; MPO₅₀₄₋₅₁₈; MPO₄₆₂₋₄₇₆; MPO₆₁₇₋₆₃₁; MPO₄₄₄₋₄₅₈; MPO₆₈₉₋₇₀₃; MPO₂₄₈₋₂₆₂; MPO₅₁₁₋₅₂₅; MPO₉₇₋₁₁₁; and MPO₆₁₆₋₆₃₀.

46. An embodiment of the composition of any one of embodiments 1 to 45, further comprising a pharmaceutically acceptable stabilizer, excipient, diluent, or combination thereof.

47. An embodiment of the composition of any one of embodiments 1 to 46, formulated for intravenous administration.

48. An embodiment of the composition of any one of embodiments 1 to 47, for use in a method of treating an autoimmune or inflammatory disease.

49. Use of the composition of any one of embodiments 1 to 47, in the manufacture of a medicament for treating an autoimmune or inflammatory disease.

50. An embodiment of the composition or use of embodiment 49, wherein the ubiquitous autoantigen or the second ubiquitous autoantigen is not a polypeptide derived from myelin basic protein, myelin associated glycoprotein, myelin oligodendrocyte protein (MOG), proteolipid protein, oligodendrocyte myelin oligoprotein, myelin associated oligodendrocyte basic protein, oligodendrocyte specific protein, heat shock proteins, an oligodendrocyte specific protein, NOGO A, glycoprotein Po, peripheral myelin protein 22, or 2′3′-cyclic nucleotide 3′-phosphodiesterase.

51. An embodiment of the composition or use of embodiment 49, wherein the ubiquitous autoantigen or the second ubiquitous autoantigen is not a polypeptide derived from pre-proinsulin, proinsulin, islet-specific glucose-6-phosphatase (IGRP), glutamate decarboxylase (GAD), islet cell autoantigen-2 (ICA2), or insulin.

TABLE 5
additional ubiquitous autoantigens

| Cellular | | | |
|---|---|---|---|
| location | Uniprot Code | | Description |
| Cytoplasm | P14152 | Mdh1 | Malate dehydrogenase, cytoplasmic |
| | | | (EC 1.1.1.37) (Cytosolic |
| | | | malate dehydrogenase) |
| Cytoplasm | P63260 | Actg1 | Actin, cytoplasmic 2 (Gamma-actin) |
| | | | [Cleaved into: Actin, cytoplasmic 2, |
| | | | N-terminally processed] |
| Cytoplasm | P20152 | Vim | Vimentin |
| Cytoplasm | P06151 | Ldha | L-lactate dehydrogenase A chain |
| | | | (LDH-A) (EC 1.1.1.27) |
| | | | (LDH muscle subunit) (LDH-M) |
| Cytoplasm | P16858 | Gapdh | Glyceraldehyde-3-phosphate dehydrogenase |
| | | | (GAPDH) (EC 1.2.1.12) (Peptidyl- |
| | | | cysteine S-nitrosylase GAPDH) |
| | | | (EC 2.6.99.—) |
| Cytoplasm | P63101 | Ywhaz | 14-3-3 protein zeta/delta |
| | | | (Protein kinase C inhibitor |
| | | | protein 1) (KCIP-1) (SEZ-2) |
| Cytoplasm | P11404 | Fabp3 | Fatty acid-binding protein, heart |
| | | | (Fatty acid-binding protein 3) (Heart- |
| | | | type fatty acid-binding protein) (H-FABP) |
| | | | (Mammary-derived growth inhibitor) (MDGI) |
| Cytoplasm | O08997 | Atox1 | Copper transport protein ATOX1 |
| | | | (Metal transport protein ATX1) |
| Cytoplasm | P35700 | Prdx1 | Peroxiredoxin-1 (EC 1.11.1.15) |
| | | | (Macrophage 23 kDa stress protein) |
| | | | (Osteoblast-specific factor 3) (OSF-3) |
| | | | (Thioredoxin peroxidase 2) (Thioredoxin- |
| | | | dependent peroxide reductase 2) |
| Cytoplasm | Q9CQM5 | Txndc17 | Thioredoxin domain-containing protein 17 |
| | | | (14 kDa thioredoxin-related protein) |
| | | | (TRP14) (Protein 42-9-9) |
| | | | (Thioredoxin-like protein 5) |
| Nucleus | P09405 | Ncl | Nucleolin (Protein C23) |
| Nucleus | Q9Z2X1 | Hnrnpf | Heterogeneous nuclear ribonucleoprotein |
| | | | F (hnRNP F) [Cleaved into: Heterogeneous |
| | | | nuclear ribonucleoprotein F, N- |
| | | | terminally processed] |
| Nucleus | Q3U898 | Cops9 | COP9 signalosome complex |
| | | | subunit 9 |
| Nucleus | P62322 | Lsm5 | U6 snRNA-associated Sm-like |
| | | | protein LSm5 |
| Nucleus | P17918 | Pcna | Proliferating cell nuclear antigen |
| | | | (PCNA) (Cyclin) |
| Nucleus | O88569 | Hnrnpa2b1 | Heterogeneous nuclear ribonucleoproteins |
| | | | A2/B1 (hnRNP A2/B1) |
| Nucleus | P40142 | Tkt | Transketolase (TK) |
| | | | (EC 2.2.1.1) (P68) |
| Nucleus | Q60972 | Rbbp4 | Histone-binding protein RBBP4 |
| | | | (Chromatin assembly factor 1 subunit C) |
| | | | (CAF-1 subunit C) (Chromatin assembly |
| | | | factor I p48 subunit) (CAF-I 48 |
| | | | kDa subunit) (CAF-I p48) (Nucleosome- |
| | | | remodeling factor subunit RBAP48) |
| | | | (Retinoblastoma-binding protein 4) |
| | | | (RBBP-4) (Retinoblastoma-binding |
| | | | protein p48) |
| Nucleus | Q60973 | Rbbp7 | Histone-binding protein RBBP7 (Histone |
| | | | acetyltransferase type B subunit 2) |
| | | | (Nucleosome-remodeling factor |
| | | | subunit RBAP46) (Retinoblastoma- |
| | | | binding protein 7) (RBBP-7) |
| | | | (Retinoblastoma-binding |
| | | | protein p46) |
| Nucleus | P15532 | Nme1 | Nucleoside diphosphate kinase A (NDK A) |
| | | | (NDP kinase A) (EC 2.7.4.6) |
| | | | (Metastasis inhibition factor |
| | | | NM23) (NDPK-A) (Tumor metastatic |
| | | | process-associated protein) |
| | | | (nm23-M1) |
| Plasma | More Abundant | | |
| Membrane | | | |
| Plasma | P68040 | Rack1 | Receptor of activated protein C kinase |
| Membrane | | | 1 (12-3) (Guanine nucleotide-binding |
| | | | protein subunit beta-2-like 1) |
| | | | (Receptor for activated C kinase) |
| | | | (Receptor of activated protein |
| | | | kinase C 1) (p205) [Cleaved into: |
| | | | Receptor of activated protein C |
| | | | kinase 1, N-terminally processed (Guanine |
| | | | nucleotide-binding protein subunit |
| | | | beta-2-like 1, N-terminally processed)] |
| Plasma | Q62351 | Tfrc | Transferrin receptor protein 1 |
| Membrane | | | (TR) (TfR) (TfR1) (Trfr) |
| | | | (CD antigen CD71) |
| Plasma | Q9QYY0 | Gab1 | GRB2-associated-binding protein 1 |
| Membrane | | | (GRB2-associated binder 1) |
| | | | (Growth factor receptor bound protein 2- |
| | | | associated protein 1) |
| Plasma | P42703 | Lifr | Leukemia inhibitory factor receptor |
| Membrane | | | (LIF receptor) (LIF-R) |
| | | | (D-factor/LIF receptor) |
| | | | (CD antigen CD118) |
| Plasma | Q01279 | Egfr | Epidermal growth factor receptor |
| Membrane | | | (EC 2.7.10.1) |
| Plasma | Q62351 | Tfrc | Transferrin receptor protein 1 |
| Membrane | | | (TR) (TfR) (TfR1) (Trfr) |
| | | | (CD antigen CD71) |
| Plasma | P14069 | S100a6 | Protein S100-A6 (5B10) (Calcyclin) |
| Membrane | | | (Prolactin receptor-associated protein) |
| | | | (S100 calcium-binding protein A6) |
| Plasma | Q61160 | Fadd | FAS-associated death domain protein |
| Membrane | | | (FAS-associating death domain- |
| | | | containing protein) (Mediator of |
| | | | receptor induced toxicity) |
| | | | (Protein FADD) |
| Plasma | Q5M8N0 | Cnrip1 | CB1 cannabinoid receptor-interacting |
| Membrane | | | protein 1 (CRIP-1) |
| Plasma | Q60902 | Eps1511 | Epidermal growth factor receptor |
| Membrane | | | substrate 15-like 1 (Epidermal growth |
| | | | factor receptor pathway substrate 15- |
| | | | related sequence) (Eps15-rs) |
| | | | (Eps15-related protein) (Eps15R) |
| Plasma | P97300 | Nptp | Nuroplastin |
| Membrane | | | |
| Mitochondria | Q64433 | Hspe1 | 10 kDa heat shock protein, mitochondrial |
| | | | (Hsp10) (10 kDa chaperonin) |
| | | | (Chaperonin 10) (CPN10) |
| Mitochondria | Q07813 | Bax | Apoptosis regulator BAX |
| Mitochondria | P38647 | Hspa9 | Stress-70 protein, mitochondrial |
| | | | (75 kDa glucose-regulated protein) |
| | | | (GRP-75) (Heat shock 70 kDa |
| | | | protein 9) (Mortalin) (Peptide-binding |
| | | | protein 74) (PBP74) (p66 MOT) |
| Mitochondria | P19157 | Gstp1 | Glutathione S-transferase P 1 |
| | | | (Gst P1) (EC 2.5.1.18) (GST YF-YF) |
| | | | (GST class-pi) (GST-piB) (Preadipocyte |
| | | | growth factor) |
| Mitochondria | Q9CR21 | Ndufab1 | Acyl carrier protein, mitochondrial |
| | | | (ACP) (CI-SDAP) (NADH-ubiquinone |
| | | | oxidoreductase 9.6 kDa subunit) |
| Mitochondria | P08249 | Mdh2 | Malate dehydrogenase, mitochondrial |
| | | | (EC 1.1.1.37) |
| Mitochondria | P63038 | Hspd1 | 60 kDa heat shock protein, mitochondrial |
| | | | (EC 3.6.4.9) (60 kDa chaperonin) |
| | | | (Chaperonin 60) (CPN60) (HSP-65) |
| | | | (Heat shock protein 60) (HSP-60) |
| | | | (Hsp60) (Mitochondrial matrix protein P1) |
| Mitochondria | Q03265 | Atp5f1a | ATP synthase subunit alpha, mitochondrial |
| | | | (ATP synthase F1 subunit alpha) |
| Mitochondria | P63038 | Hspd1 | 60 kDa heat shock protein, mitochondrial |
| | | | (EC 3.6.4.9) (60 kDa chaperonin) |
| | | | (Chaperonin 60) (CPN60) (HSP-65) |
| | | | (Heat shock protein 60) (HSP-60) |
| | | | (Hsp60) (Mitochondrial matrix protein P1) |
| Mitochondria | P56382 | Atp5f1e | ATP synthase subunit epsilon, mitochondrial |
| | | | (ATPase subunit epsilon) (ATP synthase |
| | | | F1 subunit epsilon) |
| Golgi | More Abundant | | |
| Golgi | P61205 | Arf3 | ADP-ribosylation factor 3 |
| Golgi | P61750 | Arf4 | ADP-ribosylation factor 4 |
| Golgi | P84084 | Arf5 | ADP-ribosylation factor 5 |
| Golgi | Q99LT0 | Dpy30 | Protein dpy-30 homolog (Dpy-30-like |
| | | | protein) (Dpy-30L) |
| Golgi | P53811 | Pitpnb | Phosphatidylinositol transfer protein |
| | | | beta isoform (PI-TP-beta) (PtdIns |
| | | | transfer protein beta) (PtdlnsTP beta) |
| Golgi | O35643 | Ap1b1 | AP-1 complex subunit beta-1 (Adaptor |
| | | | protein complex AP-1 subunit beta-1) |
| | | | (Adaptor-related protein complex 1 |
| | | | subunit beta-1) (Beta-1-adaptin) |
| | | | (Beta-adaptin 1) (Clathrin assembly |
| | | | protein complex 1 beta large chain) |
| | | | (Golgi adaptor HA1/AP1 adaptin |
| | | | beta subunit) |
| Golgi | P61211 | Arl1 | ADP-ribosylation factor-like protein 1 |
| Golgi | Q3UPH1 | Prrc1 | Protein PRRC1 (Proline-rich and coiled- |
| | | | coil-containing protein 1) |
| Golgi | P61924 | Copz1 | Coatomer subunit zeta-1 (Zeta-1-coat protein) |
| | | | (Zeta-1 COP) |
| Golgi | Q9CQC9 | Sar1b | GTP-binding protein SAR1b |
| ER | O55022 | Pgrmc1 | Membrane-associated progesterone receptor |
| | | | component 1 (mPR) |
| ER | P33267 | Cyp2f2 | Cytochrome P450 2F2 (EC 1.14.14.—) (CYPIIF2) |
| | | | (Cytochrome P450-NAH-2) (Naphthalene |
| | | | dehydrogenase) (Naphthalene hydroxylase) |
| ER | O55143 | Atp2a2 | Sarcoplasmic/endoplasmic reticulum calcium |
| | | | ATPase 2 (SERCA2) (SR Ca(2+)-ATPase 2) |
| | | | (EC 3.6.3.8) (Calcium pump 2) |
| | | | (Calcium-transporting ATPase sarcoplasmic |
| | | | reticulum type, slow twitch skeletal muscle |
| | | | isoform) (Endoplasmic reticulum class 1/2 |
| | | | Ca(2+) ATPase) |
| ER | P45878 | Fkbp2 | Peptidyl-prolyl cis-trans isomerase FKBP2 |
| | | | (PPIase FKBP2) (EC 5.2.1.8) (13 kDa FK506- |
| | | | binding protein) (13 kDa FKBP) (FKBP-13) |
| | | | (FK506-binding protein 2) (FKBP-2) |
| | | | (Immunophilin FKBP13) (Rotamase) |
| ER | P56395 | Cyb5a | Cytochrome b5 |
| ER | Q9D1Q6 | Erp44 | Endoplasmic reticulum resident protein 44 |
| | | | (ER protein 44) (ERp44) (Thioredoxin domain- |
| | | | containing protein 4) |
| ER | P35564 | Canx | Calnexin |
| ER | P08113 | Hsp90b1 | Endoplasmin (94 kDa glucose-regulated |
| | | | protein) (GRP-94) (Endoplasmic reticulum |
| | | | resident protein 99) (ERp99) (Heat shock |
| | | | protein 90 kDa beta member 1) (Polymorphic |
| | | | tumor rejection antigen 1) (Tumor rejection |
| | | | antigen gp96) |
| ER | Q01853 | Vcp | Transitional endoplasmic reticulum ATPase |
| | | | (TER ATPase) (EC 3.6.4.6) |
| | | | (15S Mg(2+)-ATPase p97 subunit) |
| | | | (Valosin-containing protein) (VCP) |
| | Q9D0F3 | Lman1 | Protein ERGIC-53 (ER-Golgi intermediate |
| | | | compartment 53 kDa protein) |
| | | | (Lectin mannose-binding 1) (p58) |

Also contemplated are the human proteins or human homologues disclosed in this table

SEQUENCES DISCLOSED HEREIN

| HLA | Position in protein | | Amino Acid Sequence | SEQ. ID |
|---|---|---|---|---|
| Allele bound | start | end | PDC-E2 derived peptides | NO: |
| DRB3*0202 | 353 | 367 | GRVFVSPLAKKLAVE | 1 |
| DRB3*0202 | 72 | 86 | RLLLQLLGSPGRRYY | 2 |
| DRB3*0202 | 422 | 436 | DIPISNIRRVIAQRL | 3 |
| DRB5*0101 | 353 | 367 | GRVFVSPLAKKLAVE | 4 |
| DRB5*0101 | 80 | 94 | SPGRRYYSLPPHQKV | 5 |
| DRB5*0101 | 535 | 549 | ETIANDVVSLATKAR | 6 |
| DRB4*0101 | 629 | 643 | AQWLAEFRKYLEKPI | 7 |
| DRB4*0101 | 122 | 135 | GDLIAEVETDKATV | 8 |
| DRB4*0101 | 249 | 262 | GDLLAEIETDKATI | 9 |
| DRB1*0801 | 249 | 263 | GDLLAEIETDKATIG | 10 |
| DRB3*0202 | 422 | 436 | DIPISNIRRVIAQRL | 11 |
| DRB5*0101 | 80 | 94 | SPGRRYYSLPPHQKV | 12 |

CYP2D6 derived peptides

| | | | | |
|---|---|---|---|---|
| DRB3*01:01 | 284 | 298 | GNPESSFNDENLRIV | 13 |
| DRB3*01:01 | 289 | 303 | SFNDENLRIVVADLF | 14 |
| DRB3*01:01 | 318 | 332 | LLLMILHPDVQRRVQ | 15 |
| DRB1*03:01 | 313 | 332 | TLAWGLLLMILHPDVQRRVQ | 16 |
| DRB1*03:01 | 393 | 412 | TTLITNLSSVLKDEAVWEKP | 17 |
| DRB1*03:01 | 192 | 206 | GRRFEYDDPRFLRLL | 18 |
| DRB1*03:01 | 5 | 19 | ALVPLAVIVAIFLLL | 19 |
| DRB1*03:01 | 293 | 307 | ENLRIVVADLFSAGM | 20 |
| DRB3*0202 | 219 | 233 | FLREVLNAVPVLLHI | 21 |
| DRB3*0202 | 237 | 251 | AGKVLRFQKAFLTQL | 22 |
| DRB3*0202 | 15 | 29 | IFLLLVDLMHRRQRW | 23 |
| DRB4*0101 | 235 | 249 | ALAGKVLRFQKAFLT | 24 |
| DRB4*0101 | 317 | 331 | GLLLMILHPDVQRRV | 25 |
| DRB4*0101 | 293 | 307 | ENLRIVVADLFSAGM | 26 |
| DRB5*0101 | 428 | 442 | VKPEAFLPFSAGRRA | 27 |
| DRB5*0101 | 237 | 251 | AGKVLRFQKAFLTQL | 28 |
| DRB5*0101 | 14 | 28 | AIFLLLVDLMHRRQR | 29 |
| DRB1*04:01 | 199 | 213 | DPRFLRLLDLAQEGL | 30 |
| DRB1*04:01 | 450 | 464 | RMELFLFFTSLLQHF | 31 |
| DRB1*04:01 | 301 | 315 | DLFSAGMVTTSTTLA | 32 |
| DRB1*07:01 | 452 | 466 | ELFLFFTSLLQHFSF | 33 |
| DRB1*07:01 | 59 | 73 | DQLRRRFGDVFSLQL | 34 |
| DRB1*07:01 | 130 | 144 | EQRRFSVSTLRNLGL | 35 |
| DRB1*07:01 | 193 | 212 | RRFEYDDPRFLRLLDLAQEG | 36 |
| DRB1*07:01 | 305 | 324 | AGMVTTSTTLAWGLLLMILH | 37 |

ACTB derived peptides

| | | | | |
|---|---|---|---|---|
| DRB1*0301 | 202 | 216 | TTAEREIVRDIKEKL | 38 |
| DRB1*0301 | 170 | 184 | ALPHAILRLDLAGRD | 39 |
| DRB1*0301 | 245 | 259 | SGRTTGIVMDSGDGV | 40 |
| DRB3*02:02 | 187 | 201 | DYLMKILTERGYSFT | 41 |
| DRB3*02:02 | 172 | 186 | PHAILRLDLAGRDLT | 42 |
| DRB3*02:02 | 131 | 145 | AMYVAIQAVLSLYAS | 43 |
| DRB4*0101 | 131 | 145 | AMYVAIQAVLSLYAS | 44 |
| DRB4*0101 | 171 | 185 | LPHAILRLDLAGRDL | 45 |
| DRB4*0101 | 129 | 143 | TPAMYVAIQAVLSLY | 46 |
| DRB5*0101 | 164 | 178 | PIYEGYALPHAILRL | 47 |
| DRB5*0101 | 25 | 39 | DAPRAVFPSIVGRPR | 48 |
| DRB5*0101 | 323 | 337 | STMKIKIIAPPERKY | 49 |
| DRB3*0101 | 146 | 160 | GRTTGIVMDSGDGVT | 50 |
| DRB3*0101 | 18 | 32 | KAGFAGDDAPRAVFP | 51 |
| DRB4*0101 | 171 | 185 | LPHAILRLDLAGRDL | 52 |

SLA derived peptides

| | | | | |
|---|---|---|---|---|
| DRB1*0301 | 334 | 348 | YKKLLKERKEMFSYL | 53 |
| DRB1*0301 | 196 | 210 | DELRTDLKAVEAKVQ | 54 |
| DRB1*0301 | 115 | 129 | NKITNSLVLDIIKLA | 55 |
| DRB1*0301 | 373 | 386 | NRLDRCLKAVRKER | 56 |
| DRB1*0301 | 186 | 197 | LIQQGARVGRID | 57 |
| DRB3*0202 | 342 | 356 | KEMFSYLSNQIKKLS | 58 |
| DRB3*0202 | 110 | 124 | GSSLLNKITNSLVLD | 59 |
| DRB3*0202 | 299 | 313 | NDSFIQEISKMYPGR | 60 |
| DRB4*0101 | 49 | 63 | STLELFLHELAIMDS | 61 |
| DRB4*0101 | 260 | 274 | SKCMHLIQQGARVGR | 62 |
| DRB4*0101 | 119 | 133 | NSLVLDIIKLAGVHT | 63 |
| DRB5*0101 | 86 | 100 | RRHYRFIHGIGRSGD | 64 |
| DRB5*0101 | 26 | 40 | RSHEHLIRLLLEKGK | 65 |
| DRB5*0101 | 331 | 345 | SNGYKKLLKERKEMF | 66 |
| DRB1*0401 | 317 | 331 | SPSLDVLITLLSLGS | 67 |
| DRB1*0401 | 171 | 185 | DQKSCFKSMITAGFE | 68 |
| DRB1*0401 | 417 | 431 | YTFRGFMSHTNNYPC | 69 |
| DRB1*0701 | 359 | 373 | YNERLLHTPHNPISL | 70 |
| DRB1*0701 | 215 | 229 | DCILCIHSTTSCFAP | 71 |
| DRB1*0701 | 111 | 125 | SSLLNKITNSLVLDI | 72 |

FTCD derived peptides

| | | | | |
|---|---|---|---|---|
| DRB3*0202 | 439 | 453 | ALQEGLRRAVSVPLT | 73 |
| DRB3*0202 | 381 | 395 | RRQFQSLDTTMRRLI | 74 |
| DRB3*0202 | 297 | 311 | EQRIRLVVSRLGLDS | 75 |
| DRB1*0301 | 525 | 539 | AKTQAALVLDCLETR | 76 |
| DRB1*0301 | 218 | 232 | KVQGIGWYLDEKNLA | 77 |
| DRB1*0301 | 495 | 509 | YFNVLINLRDITDEA | 78 |
| DRB4*0101 | 262 | 276 | LPVVGSQLVGLVPLK | 79 |
| DRB4*0101 | 300 | 314 | IRLVVSRLGLDSLCP | 80 |
| DRB4*0101 | 259 | 273 | ELSLPVVGSQLVGLV | 81 |
| DRB5*0101 | 490 | 504 | GVFGAYFNVLINLRD | 82 |
| DRB5*0101 | 389 | 403 | TTMRRLIPPFREASA | 83 |
| DRB5*0101 | 295 | 309 | EEEQRIRLVVSRLGL | 84 |
| DRB3*0101 | 271 | 285 | GLVPLKALLDAAAFY | 85 |
| DRB3*0101 | 498 | 512 | VLINLRDITDEAFKD | 86 |
| DRB3*0101 | 301 | 315 | RLVVSRLGLDSLCPF | 87 |

MPO derived peptides

| | | | | |
|---|---|---|---|---|
| DRB3*0202 | 322 | 336 | SNITIRNQINALTSF | 88 |
| DRB3*0202 | 714 | 728 | KNNIFMSNSYPRDFV | 89 |
| DRB3*0202 | 617 | 631 | LGTVLRNLKLARKLM | 90 |
| DRB1*0301 | 504 | 518 | LIQPFMFRLDNRYQP | 91 |
| DRB1*0301 | 462 | 476 | YLPLVLGPTAMRKYL | 92 |
| DRB1*0301 | 617 | 631 | LGTVLRNLKLARKLM | 93 |
| DRB4*0101 | 444 | 458 | QEARKIVGAMVQIIT | 94 |
| DRB4*0101 | 689 | 703 | QQRQALAQISLPRII | 95 |
| DRB4*0101 | 248 | 262 | RSLMFMQWGQLLDHD | 96 |
| DRB5*0101 | 511 | 525 | RLDNRYQPMEPNPRV | 97 |
| DRB5*0101 | 97 | 111 | ELLSYFKQPVAATRT | 98 |
| DRB5*0101 | 616 | 630 | QLGTVLRNLKLARKL | 99 |

Any of the peptides described herein may encompass a naturally occurring variant that does not affect binding to an MHC class II molecule or recognition by a T cell receptor. Such variants are contemplated herein and included with reference to the sequence.

| SEQ | |
|---|---|
| ID | |
| NO: | |
| 103 | MAIIYLILLFTAVRGIKEEHVIIQAEFYLNPDQSGEFMFDFD |
| | GDEIFHVDMAKKETVWRLEEFGRFASFEAQGALANIAVDKAN |
| | LEIMTKRSNYTPITNVPPEVTVLTNSPVELREPNVLICFIDK |
| | FTPPVVNVTWLRNGKPVTTGVSETVFLPREDHLFRKFHYLPF |
| | LPSTEDVYDCRVEHWGLDEPLLKHWEFDAPSPLPETTESGGG |
| | GGDKTHTCPPCPAPEAAGGPSVFLEPPKPKDTLMISRTPEVT |
| | CVVVDVSHEDPEVKFNWYVDGVEVHNAKTKPREEQYNSTYRV |
| | VSVLTVLHQDWLNGKEYKCKVSNKALPAPIEKTISKAKGQPR |
| | EPQVYTLPPCRDELTKNQVSLWCLVKGFYPSDIAVEWESNGQ |
| | PENNYKTTPPVLDSDGSFFLYSKLTVDKSRWQQGNVFSCSVM |
| | HEALHNHYTQKSLSLSPGKGSGSGSGSC |
| 104 | MGSLQPLATLYLLGMLVASSLGGDLIAEVETDKATVGGGGGS |
| | GGGSGGSGDTQPRFLEQAKCECHFLNGTERVWNLIRYIYNQE |
| | EYARYNSDLGEYQAVTELGRPDAEYWNSQKDLLERRRAEVDT |
| | YCRYNYGVVESFTVQRRVQPKVTVYPSKTQPLQHHNLLVCSV |
| | NGFYPGSIEVRWFRNSQEEKAGVVSTGLIQNGDWTFQTLVML |
| | ETVPRSGEVYTCQVEHPSMMSPLTVQWSARSESAQSKSGGGG |
| | GDKTHTCPPCPAPEAAGGPSVFLEPPKPKDTLMISRTPEVTC |
| | VVVDVSHEDPEVKFNWYVDGVEVHNAKTKPREEQYNSTYRVV |
| | SVLTVLHQDWLNGKEYKCKVSNKALPAPIEKTISKAKGQPRE |
| | PQVCTLPPSRDELTKNQVSLSCAVKGFYPSDIAVEWESNGQP |
| | ENNYKTTPPVLDSDGSFFLVSKLVDKSRWQQGNVFSCSVMHE |
| | ALHNHYTQKSLSLSPGK |

1. A composition comprising:
a) a plurality of antigen-major histocompatibility complexes (antigen-MHCs), each antigen-MHC of the plurality comprising an autoantigen associated with a binding groove of an MHC molecule; and
b) a nanoparticle core possessing a diameter of between 1 and about 100 nanometers; wherein the antigen-MHCs are coupled to the nanoparticle core or a biocompatible layer surrounding the nanoparticle core, wherein each antigen-MHC complex is a heterodimer comprising an alpha chain and a beta chain, wherein:
a. the alpha chain comprises an amino acid sequence having at least 90%, 95%, 97%, 98%, 99%, or 100% sequence identity to SEQ ID NO: 103, and
b. the beta chain comprises an amino acid sequence having at least 90%, 95%, 97%, 98%, 99%, or 100% sequence identity to SEQ ID NO:104.

2. The composition of claim 1, wherein the MHC molecule is an MHC class II molecule.

3. The composition of claim 1, wherein the nanoparticle core is a metal or metal oxide.

4. The composition of claim 3, wherein the metal is iron.

5. The composition of claim 3, wherein the metal oxide is iron oxide.

6. The composition of claim 1, wherein the diameter is between about 5 nanometers and about 50 nanometers.

7. The composition of claim 6, wherein the diameter is between about 5 nanometers and about 25 nanometers.

8. The composition of claim 1, wherein the plurality of antigen-MHCs is coupled to the nanoparticle core at an antigen-MHC to nanoparticle core ratio of at least 10:1.

9. The composition of claim 1, wherein the plurality of antigen-MHCs is coupled to the nanoparticle core at an antigen-MHC to nanoparticle core ratio of no more than about 150:1.

10. The composition of claim 1, wherein the plurality of antigen-MHCs is coupled to the nanoparticle core at a density from about 0.4 to about 13 antigen-MHCs per 100 nm² of nanoparticle core surface area.

11. The composition of claim 1, wherein the antigen-MHCs are covalently coupled to the nanoparticle core.

12. The composition of claim 1, wherein the antigen-MHCs are coupled to the nanoparticle core by a polyethylene glycol (PEG) linker having a mass of less than about 5 kilodaltons.

13. The composition of claim 1, wherein the nanoparticle core further comprises a biocompatible coating.

14-52. (canceled)

53. A method of treating an autoimmune or inflammatory disease in an individual comprising administering to the individual a composition according to claim 1.

54-101. (canceled)

102. The method of claim 53, wherein the autoimmune or inflammatory disease is chosen from type I diabetes, multiple sclerosis, relapsing-remitting multiple sclerosis, pemphigus, pemphigus foliaceus, pemphigus vulgaris, neuromyelitis optica spectrum disorder, rheumatoid arthritis, osteoarthritis, psoriatic arthritis, inflammatory bowel disease, Crohn's disease, celiac disease, allergic asthma, systemic lupus erythematosus, atherosclerosis, chronic obstructive pulmonary disease, emphysema, psoriasis, uveitis, Sjogren's Syndrome, scleroderma, anti-phospholipid syndrome, ANCA-associated vasculitis, primary biliary cirrhosis, autoimmune hepatitis, primary sclerosing cholangitis, and Stiff Man Syndrome.

103. (canceled)

104. (canceled)

105. The method of claim 102, wherein the autoimmune or inflammatory disease is primary biliary cirrhosis (PBC).

106. The method of claim 102, wherein the autoimmune or inflammatory disease is chosen from primary sclerosing cholangitis (PSC) and autoimmune hepatitis (AIH).

107. A polypeptide comprising an amino acid sequence having at least 90%, 95%, 97%, 98%, 99%, or 100% sequence identity to SEQ ID NO:103.

108. A polypeptide comprising an amino acid sequence having at least 90%, 95%, 97%, 98%, 99%, or 100% sequence identity to SEQ ID NO:104.

109. A heterodimer comprising an alpha chain polypeptide and a beta chain polypeptide, the alpha chain polypeptide comprising an amino acid sequence having at least 90%, 95%, 97%, 98%, 99%, or 100% sequence identity to SEQ ID NO:103, and the beta chain polypeptide comprising an amino acid sequence having at least 90%, 95%, 97%, 98%, 99%, or 100% sequence identity to SEQ ID NO:104.

110. A process for producing a polypeptide comprising a first amino acid sequence having at least 90%, 95%, 97%, 98%, 99%, or 100% sequence identity to SEQ ID NO:103 and a polypeptide comprising an amino acid sequence having at least 90%, 95%, 97%, 98%, 99%, or 100% sequence identity to SEQ ID NO:104, or a heterodimer comprising the first polypeptide and the second polypeptide, comprising:
a) preparing lentivirus encoding a monocistronic message comprising the first amino acid sequence and the second amino acid sequence separated by a P2A sequence;
b) transduced CHO-S cells with the lentivirus; and
c) purifying the first and second polypeptides, or the heterodimers comprising them, from the supernatant of the CHO-S cells.
